SEQUENCE LISTING (11 sequences)

SEQ ID NO: 1 - protein, 724 aa, Escherichia coli
MLRWKRCIMLTFISGAAFAAPEINVKQNESLPDLGSQAAQQDEQTNKGKSLKERGADYVINSATQGFENLTPEALESQARSYLQSQITSTAQSYIEDTLSPYGKVRSNLSIGQGGDLDGSSIDYFVPWYDNQTTVYFSQFSAQRKEDRTIGNIGLGVRHNFDKWLLGGNIFYDYDFTRGHRRLGIGAEAWTDYLKFSGNYYHPLSDWKDSEDFDFYEERPASGWDIRAEAWLPAYPQLGGKIVFEHYYGDEVALFGTDNLEKDPFAVTLGVKYQPVPLIAVGTDFKAGTGDNTDTTVNATLNYQFGVPLKDQLDPDKVSAAHSLMGSRHDFVERNNFIVLEYKEKDPLDVTLWLKADAMNEHPECVIKDTPEEAIGLEKCKWTINALVNHHYKIVAASWQAKNNAARTLVMPVIKENTLTEGNNNHWNLVLPVWQYSSDKAEQGKLNTWRVRLALKDEKGNRQNSGVVEITVQQDRKIELIVNNIANVPEENNHSHEASAQADGVDGVVMDLDITDSFGDNTDRNGNVLPQDNLNPQLFDANDKKVTLTNNPCTTETPCVFIAKQDKEKGTVTLSSTLPGTFRWKAKAAPYDDSNYVDVTFLGADIGGLNAFIYRVGAAKSVNLIGNKEPLPLNSRYRFVLWRDANKDGVFQLSEKLTDEEMKQYDYQWEFTGHSVNGNTGAQANTTNVDIEIPATNKEAAAKFSAQVTDGVQGYGLQVNYSKT

SEQ ID NO: 2 - DNA, 834 bp, Escherichia coli
  source 1..834: mol_type = genomic DNA
gtcatcaactccgccacgcaagggtttgaaaacttgacccctgaggcgctggaatctcaggccagaagctatctgcaaagtcaaatcacctcaaccgcacaatcttatattgaagacacactctctccctacggtaaggtccgttcgaacctctccattggtcagggcggcgatctggatggcagttccatcgattattttgttccctggtacgataatcaaaccactgtttatttcagccaattttctgcgcaacgaaaagaagatcgtacgatcggaaatatagggcttggtgtaagacataattttgataaatggttattgggtggaaatatattttatgattatgattttacccgtggacatcgccgtttaggtataggcgctgaagcgtggactgattatttaaaattttctggtaactattatcatccgctttctgactggaaagactctgaagatttcgacttttatgaagaacgccctgcgagcggttgggatattcgtgccgaagcctggcttcccgcttatccgcagcttggcggtaaaatcgttttcgaacattattacggggatgaagttgcgctatttggtaccgacaatctagagaaagatcccttcgctgtcacgttaggcgtgaaataccagcctgttccgctcattgctgtaggaacggacttcaaagcaggaaccggtgataacaccgataccacggtcaacgcaacgcttaactaccagtttggcgtcccgctcaaagaccagcttgatcctgacaaagtgagcgcggctcactcactcatgggcagccgtcacgactttgttgaacgtaacaac

SEQ ID NO: 3 - DNA, 369 bp, Escherichia coli
  source 1..369: mol_type = genomic DNA
gtcactttgtggctgaaagctgatgcgatgaatgaacatcccgagtgcgtgattaaagatacgccagaagaagctatcgggctggaaaaatgtaaatggaccattaacgcgcttgtcaaccaccactacaaaattgtcgctgcgtcatggcaggcgaaaaacaatgccgcccgcacgctggtgatgccggttatcaaagagaataccctgacagagggcaacaataaccactggaacctggtgctgcccgtctggcagtacagctctgacaaagccgaacaaggaaaactcaatacctggcgggttcgcctggcgctgaaagatgaaaagggcaaccgacagaactcaggcgtggtggaaatcaccgtt

SEQ ID NO: 4 - DNA, 309 bp, Escherichia coli
  source 1..309: mol_type = genomic DNA
agtgcacaggctgatggtgtagatggtgtggtgatggatctcgatattactgatagctttggcgacaacactgaccgcaacggtaatgtcttgccgcaggataatctcaacccgcagctgtttgatgccaacgataaaaaagtgacgctgacgaataacccctgcactaccgagactccctgtgtctttatcgccaaacaagataaagaaaaggggaccgttacgctctccagtacgttgcctggcaccttccgttggaaggcgaaagctgccccctatgatgacagtaactatgttgacgtaacgttc

SEQ ID NO: 5 - DNA, 357 bp, Escherichia coli
  source 1..357: mol_type = genomic DNA
ggcgggctgaatgcctttatctatcgcgttggtgcggctaaatccgtgaatcttatcggcaataaagaaccgttgcccctcaatagccgatatcgctttgtgttgtggcgcgacgctaacaaagacggtgtgttccagctatctgaaaagctcactgacgaagagatgaaacagtatgactaccaatgggaattcacggggcatagcgtaaatggcaatactggcgcgcaagccaataccactaacgtcgatatcgagatcccggcaacgaacaaggaagcggcagcgaagtttagtgcccaggtaaccgatggtgtgcaagggtacggtttgcaggtcaattacagcaagacatag

SEQ ID NO: 6 - DNA, 2175 bp, Escherichia coli
  source 1..2175: mol_type = genomic DNA
atgctgcgatggaaacgctgtattatgctaacatttatctctggtgctgctttcgcggcaccagagataaatgttaagcaaaacgaatcgttacctgatttaggtagccaggcagcacaacaggatgaacaaaccaacaagggtaaatcgctgaaagagcgcggagccgattacgtcatcaactccgccacgcaagggtttgaaaacttgacccctgaggcgctggaatctcaggccagaagctatctgcaaagtcaaatcacctcaaccgcacaatcttatattgaagacacactctctccctacggtaaggtccgttcgaacctctccattggtcagggcggcgatctggatggcagttccatcgattattttgttccctggtacgataatcaaaccactgtttatttcagccaattttctgcgcaacgaaaagaagatcgtacgatcggaaatatagggcttggtgtaagacataattttgataaatggttattgggtggaaatatattttatgattatgattttacccgtggacatcgccgtttaggtataggcgctgaagcgtggactgattatttaaaattttctggtaactattatcatccgctttctgactggaaagactctgaagatttcgacttttatgaagaacgccctgcgagcggttgggatattcgtgccgaagcctggcttcccgcttatccgcagcttggcggtaaaatcgttttcgaacattattacggggatgaagttgcgctatttggtaccgacaatctagagaaagatcccttcgctgtcacgttaggcgtgaaataccagcctgttccgctcattgctgtaggaacggacttcaaagcaggaaccggtgataacaccgataccacggtcaacgcaacgcttaactaccagtttggcgtcccgctcaaagaccagcttgatcctgacaaagtgagcgcggctcactcactcatgggcagccgtcacgactttgttgaacgtaacaactttattgttctggaatataaagagaaagatcccctcgatgtcactttgtggctgaaagctgatgcgatgaatgaacatcccgagtgcgtgattaaagatacgccagaagaagctatcgggctggaaaaatgtaaatggaccattaacgcgcttgtcaaccaccactacaaaattgtcgctgcgtcatggcaggcgaaaaacaatgccgcccgcacgctggtgatgccggttatcaaagagaataccctgacagagggcaacaataaccactggaacctggtgctgcccgtctggcagtacagctctgacaaagccgaacaaggaaaactcaatacctggcgggttcgcctggcgctgaaagatgaaaagggcaaccgacagaactcaggcgtggtggaaatcaccgttcagcaggaccgtaaaattgagctgattgttaataacatcgcgaacgtgccggaagagaacaaccacagccacgaagccagtgcacaggctgatggtgtagatggtgtggtgatggatctcgatattactgatagctttggcgacaacactgaccgcaacggtaatgtcttgccgcaggataatctcaacccgcagctgtttgatgccaacgataaaaaagtgacgctgacgaataacccctgcactaccgagactccctgtgtctttatcgccaaacaagataaagaaaaggggaccgttacgctctccagtacgttgcctggcaccttccgttggaaggcgaaagctgccccctatgatgacagtaactatgttgacgtaacgttcctgggggctgacatcggcgggctgaatgcctttatctatcgcgttggtgcggctaaatccgtgaatcttatcggcaataaagaaccgttgcccctcaatagccgatatcgctttgtgttgtggcgcgacgctaacaaagacggtgtgttccagctatctgaaaagctcactgacgaagagatgaaacagtatgactaccaatgggaattcacggggcatagcgtaaatggcaatactggcgcgcaagccaataccactaacgtcgatatcgagatcccggcaacgaacaaggaagcggcagcgaagtttagtgcccaggtaaccgatggtgtgcaagggtacggtttgcaggtcaattacagcaagacatag

SEQ ID NO: 7 - protein, 136 aa, Escherichia coli
FIVLEYKEKDPLDVTLWLKADAMNEHPECVIKDTPEEAIGLEKCKWTINALVNHHYKIVAASWQAKNNAARTLVMPVIKENTLTEGNNNHWNLVLPVWQYSSDKAEQGKLNTWRVRLALKDEKGNRQNSGVVEITV

SEQ ID NO: 8 - protein, 129 aa, Escherichia coli
QQDRKIELIVNNIANVPEENNHSHEASAQADGVDGVVMDLDITDSFGDNTDRNGNVLPQDNLNPQLFDANDKKVTLTNNPCTTETPCVFIAKQDKEKGTVTLSSTLPGTFRWKAKAAPYDDSNYVDVTF

SEQ ID NO: 9 - protein, 123 aa, Escherichia coli
LGADIGGLNAFIYRVGAAKSVNLIGNKEPLPLNSRYRFVLWRDANKDGVFQLSEKLTDEEMKQYDYQWEFTGHSVNGNTGAQANTTNVDIEIPATNKEAAAKFSAQVTDGVQGYGLQVNYSKT

SEQ ID NO: 10 - protein, 1024 aa, Escherichia coli
MTTITTAQIKSTLQSAKQSAANKLHSAGQSTKDALKKAAEQTRNAGNRLILLIPKDYKGQGSSLNDLVRTADELGIEVQYDEKNGTAITKQVFGTAEKLIGLTERGVTIFAPQLDKLLQKYQKAGNILGGGAENIGDNLGKAGGILSTFQNFLGTALSSMKIDELIKKQKSGGNVSSSELAKASIELINQLVDTVASLNNNVNSFSQQLNTLGSVLSNTKHLNGVGNKLQNLPNLDNIGAGLDTVSGILSAISASFILSNADADTRTKAAAGVELTTKVLGNVGKGISQYIIAQRAAQGLSTSAAAAGLIASAVTLAISPLSFLSIADKFKRANKIEEYSQRFKKLGYDGDSLLAAFHKETGAIDASLTTISTVLASVSSGISAAATTSLVGAPVSALVGAVTGIISGILEASKQAMFEHVASKMADVIAEWEKKHGKNYFENGYDARHAAFLEDNFKILSQYNKEYSVERSVLITQQHWDTLIGELAGVTRNGDKTLSGKSYIDYYEEGKRLEKKXDEFQKQVFDPLKGNIDLSDSKSSTLLKFVTPLLTPGEEIRERRQSGKYEYITELLVKGVDKWTVKGVQDKGAVYDYSNLIQHASVGNNQYREIRIESHLGDGDDKVFLSAGSANIYAGKGHDVVYYDKTDTGYLTIDGTKATEAGNYTVTRVLGGDVKVLQEVVKEQEVSVGKRTEKTQYRSYEFTHINGKNLTETDNLYSVEELIGTTRADKFFGSKFTDIFHGADGDDLIEGNDGNDRLYGDKGNDTLSGGNGDDQLYGGDGNDKLIGVAGNNYLNGGDGDDEFQVQGNSLAKNVLFGGKGNDKLYGSEGADLLDGGEGDDLLKGGYGNDIYRYLSGYGHHIIDDDGGKEDKLSLADIDFRDVAFKREGNDLIMYKGEGNVLSIGHKNGITFRNWFEKESGDISNHEIEQIFDKSGRIITPDSLKKALEYQQRNNKASYVYGNDALAYGSQGDLNPLINEISKIISAAGSFDVKEERTAASLLQLSGNASDFSYGRNSITLTTSA

SEQ ID NO: 11 - DNA, 8215 bp, Escherichia coli
  source 1..8215: mol_type = genomic DNA
gataatggttcatgctaccgggcgaatgaaacacgtcagttcgccaggatgttgggacttgaaccgaagaacacggcagtgcggagtccggagagtaacggaataacagagagcttcgtgaaaacgataaagcgtgattacataagtatcatgcccaaaccagacgggttaacggcagcaaagaaccttgcagaggcgttcgagcattataacgaatggcatccgcatagtgcgctgggttatcgctcgccacgggaatatctgcggcacggggccagtaatgggttaagtgataacaggtatctggaaatataggggcaaatccacctggtcattatctggaatttgacgaagtgtgataactggtatagccagattaatctaaacctttgtctgacaaaatcagataaagaagagtagttcaaaagacaactcgtggactctcattcagagagataggcgttaccaaaatttgtttggaactgaacaagaaaattgtatttgtgtaactataatcttaatgtaaaataaaagacaccagttctgtagaatatgcttattgaagagagtgtaataataattttatatagatgttgtacaagacaggaatgagtaattatttatgcttgatgttttttgactcttgctttttatagttattatttttaagttagtcagcgcaataaaaacttgcttttaatattaatgcgagttatgacattaaacggaagaaacataaaggcatatttttgccacaatatttaatcatataatttaagttgtagtgagtttattatgaatataaacaaaccattagagattcttgggcatgtatcctggctatgggccagttctccactacacagaaactggccagtatctttgtttgcaataaatgtattacccgcaatacaggctaaccaatatgttttattaacccgggatgattaccctgtcgcgtattgtagttgggctaatttaagtttagaaaatgaaattaaatatcttaatgatgttacctcattagttgcagaagactggacttcaggtgatcgtaaatggttcattgactggattgctcctttcggggataacggtgccctgtacaaatatatgcgaaaaaaattccctgatgaactattcagagccatcagggtggatcccaaaactcatgttggtaaagtatcagaatttcatggaggtaaaattgataaacagttagcgaataaaatttttaaacaatatcaccacgagttaataactgaagtaaaaagaaagtcagattttaatttttcattaactggttaagaggtaattaaatgacaacaataaccactgcacaaattaaaagcacactgcagtctgcaaagcaatccgctgcaaataaattgcactcagcaggacaaagcacgaaagatgcattaaaaaaagcagcagagcaaacccgcaatgcgggaaacagactcattttacttatccctaaagattataaaggacagggttcaagccttaatgaccttgtcaggacggcagatgaactgggaattgaagtccagtatgatgaaaagaatggcacggcgattactaaacaggtgttcggcacagcagagaaactcattggcctcaccgaacggggagtgactatctttgcaccacaattagacaaattactgcaaaagtatcaaaaagcaggtaatatattaggcggcggtgctgaaaatatcggtgataacttaggaaaggcaggcggtatattgtcaacgtttcaaaatttcctgggtactgcactttcctcaatgaaaatagacgaactgataaagaaacaaaaatctggtggcaatgtcagttcttctgaactggcaaaagcgagtattgagctaatcaatcaacttgtggatacagttgccagccttaataataatgttaactcattttctcaacaactcaatactctgggaagtgtattatccaatacaaagcacctgaacggtgttggtaataagttacagaatttacctaatcttgataatatcggtgcagggttagatactgtatcgggtattttatctgcgatttcagcaagcttcattctgagcaatgcagatgcagataccagaactaaagctgcagcaggtgttgaattaacaacgaaagtactgggtaatgttggaaaaggtatttctcaatatattatcgcacagcgcgctgcacaggggttatctacatctgctgctgctgccggtttaattgcttctgctgtgacactagcaattagtcccctctcattcctgtccattgccgataagtttaaacgtgccaataaaatagaggagtattcacaacgattcaaaaaacttggatacgatggtgacagtttacttgctgctttccacaaagaaacaggggctattgatgcatcgttaacaacgataagcactgtactggcttcagtatcttcaggtattagtgctgctgcaacgacatctctggttggtgcaccggtaagcgcgctggtaggcgctgttacggggataatttcaggtatccttgaggcttcaaagcaggcaatgtttgaacatgttgccagtaaaatggctgatgttattgctgaatgggagaaaaaacacggcaaaaattactttgaaaatggatatgatgcccgccatgctgcatttttagaagataactttaaaatattatctcagtataataaagagtattctgttgaaagatcagtcctcattacccagcaacattgggatacgctgataggtgagttagctggtgtcaccagaaatggagacaaaacactcagtggtaaaagttatattgactattatgaagaaggaaaacgtctggagaaaaaaycggatgaattccagaagcaagtctttgacccattgaaaggaaatattgacctttctgacagcaaatcttctacgttattgaaatttgttacgccattgttaactcccggtgaggaaattcgtgaaaggaggcagtccggaaaatatgaatatattaccgagttattagtcaagggtgttgataaatggacggtgaagggggttcaggacaagggggctgtatatgattactctaacctgattcagcatgcatcagtcggtaataaccagtatcgggaaattcgtattgagtcacacctgggagacggggatgataaggtctttttatctgccggctcagccaatatctacgcaggtaaaggacatgatgttgtttattatgataaaacagacaccggttatctgaccattgatggcacaaaagcaaccgaagcgggtaattacacggtaacacgtgtacttggtggcgatgttaaggttttacaggaagttgtgaaggagcaggaggtttcagtcggaaaaagaactgaaaaaacgcaatatcggagttatgaattcactcatatcaatggtaaaaatttaacagagactgataacttatattccgtggaagaacttattgggaccacgcgtgccgacaagttttttggcagtaaatttactgatatcttccatggcgcggatggtgatgaccttatagaaggaaatgatgggaatgaccgcttatatggtgataaaggtaatgacacactgagtggtggaaacggagatgaccagctctatggcggtgatggtaacgataagttgattggggtagcaggtaataattacctgaacggcggagatggagatgatgagtttcaggttcagggaaattctcttgcaaaaaatgtattattcggtggaaaaggtaatgacaagctgtacggcagtgagggggcagatctgcttgatggtggagagggggatgatctcctgaaaggcggatatggtaatgatatttatcgttatctgtcaggatatggtcatcatattattgatgatgatggggggaaagaggataaactcagtttggctgatattgatttccgggatgtggccttcaagcgagaaggtaatgacctcatcatgtataaaggtgaaggtaatgttctttccattggtcataaaaatggtattacattcaggaactggtttgaaaaagagtcaggtgatatctctaatcacgagatagagcagatttttgataaaagtggccggataatcacacctgattcccttaaaaaggcacttgagtatcaacagcgtaataataaggcaagttatgtgtatgggaatgatgcattagcctatggaagtcagggtgatcttaatccattaattaatgaaatcagcaaaatcatttcagctgcaggtagcttcgatgttaaagaggaaagaactgcagcttctttattgcagttgtccggtaatgccagtgatttttcatatggacggaactcaataaccctgaccacatcagcataatatattaatttaaatgatagcaatcttactgggctgtgccacataagattgctattttttttggagtcataatggattcttgtcataaaattgattatgggttatacgccctggagattttagcccaataccataacgtctctgttaacccggaagaaattaaacatagatttgatacagacgggacaggtctgggattaacgtcatggttgcttgctgcgaaatctttagaactaaaggtaaaacaggtaaaaaaaacaattgatcgattaaactttatttttctgcccgcattagtctggagagaggatggacgtcattttattctgactaaaatcagcaaagaagtaaacagatatcttatttttgatttggagcagcgaaatccccgtgttctcgaacagtctgagtttgaggcgttatatcaggggcatattattcttattacttcccgttcttctgttaccgggaaactggcaaaatttgactttacctggtttattcctgccattataaaatacaggagaatatttattgaaacccttgttgtatctgtttttttacaattatttgcattaataaccccccttttttttcaggtggttatggacaaagtattagtgcacagggggttttcaacccttaatgttattactgttgccttatctgttgtagtggtgtttgagattatactcagcggtttaagaacttacatttttgcacatagtacaagtcggattgatgttgagttgggtgccaaactcttccggcatttactggcgctaccgatctcttattttgagagtcgtcgtgttggtgatactgttgcgagggtaagagaattagaccagatccgtaattttctgacaggacaggcattaacatctgttttggacttattattttcactcatattttttgcggtaatgtggtattacagcccaaagcttactctggtgatcttattttcgctgccttgttatgctgcatggtctgtttttattagccccattttgcgacgtcgccttgatgataagttttcacggaatgcggataatcaatctttcctggtggaatcagtaacggcgattaacactataaaagctatggcagtctcacctcagatgacgaacatatgggacaaacaattggcaggatatgttgctgcaggctttaaagtgacagtattagcaaccattggtcaacaaggaatacagttaatacaaaagactgttatgatcatcaacctatggttgggagcacacctggttatttccggggatttaagtattggtcagttaattgcttttaatatgcttgctggtcagattgttgcaccggttattcgccttgcacaaatctggcaggatttccagcaggttggtatatcagttacccgccttggtgatgtgcttaactctccaactgaaagttatcatgggaaactgacattgccggaaattaatggtgatatcacttttcgtaatatccggtttcgctataaacctgattctccggttattttggacaatatcaatcttagtattaagcagggggaggttattggtattgtcggacgttctggttcaggaaaaagcacattaactaaattaattcaacgtttttatattcctgaaaatggccaggtattaattgatggacatgatcttgcgttggccgatcctaactggttacgtcgtcaggtgggggttgtgttgcaggacaatgtgctgcttaatcgcagtattattgataatatttcactggctaatcctggcatgtccgtcgaaaaagttatttatgcagcgaaattagcaggtgctcatgattttatttctgaattgcgtgaggggtataacaccattgtcggggaacagggggcaggattatccggaggtcaacgtcaacgcatcgcaattgcaagggcgctggtgaacaaccctaaaatactcatttttgatgaagcaaccagtgctctggattatgagtcggagcatgtcatcatgcgcaatatgcacaaaatatgtaagggcagaacggttataatcattgctcatcgtctgtctacagtaaaaaatgcagaccgcattattgtcatggaaaaagggaaaattgttgaacagggtaaacataaggagctgctttctgaaccggaaagtttatacagttacttatatcagttacagtcagactaacagaaagaacagaagaatatgaaaacatggttaatggggttcagcgagttcctgttgcgctataaacttgtctggagtgaaacatggaaaatccggaagcaattagatactccggtacgtgaaaaggacgaaaatgaattcttacccgctcatctggaattaattgaaacgccagtatccagacggccgcgtctggttgcttattttattatggggtttctggttattgcttttattttatctgttttaggccaagtggaaattgttgccactgcaaatgggaaattaacacacagtgggcgtagtaaagaaattaaacctattgaaaactcaatagttaaagaaattatcgtaaaagaaggagagtcagtccggaaaggggatgtgttattaaagcttacagcactgggagctgaagctgatacgttaaaaacacagtcatcactgttacaggccaggctggaacaaactcggtatcaaattctgagcaggtcaattgaattaaataaactacctgaactaaagcttcctgatgagccttattttcagaatgtatctgaagaggaagtactgcgtttaacttctttgataaaagaacagttttccacatggcaaaatcagaagtatcaaaaagaactgaatttggataagaaaagagcagagcgattaacagtacttgcccgtataaaccgttatgaaaatttatcaagggttgaaaaaagccgtctggatgatttcagtagtttattgcataaacaggcaattgcaaaacatgctgtacttgagcaggagaataaatatgtcgaagcagtaaatgaattacgagtttataaatcacaactggagcaaattgagagtgagatattgtctgcaaaagaagaatatcagcttgttacgcagctttttaaaaatgaaattttagataagctaagacaaaccacagacaacattgggttattaactctggaattagcgaaaaatgaagagcgtcaacaggcttcagtaatcagggccccagtttcggtaaaagttcagcaactgaaggttcatactgaaggtggggttgttacaacagcggaaacactgatggtcatcgttccggaagatgacacgctggaggttactgctctggtacaaaataaagatattggttttattaacgtcgggcagaatgccatcattaaagtggaggcatttccttatacacgatatggttatctggtgggtaaggtgaaaaatataaatttagatgcaatagaagaccagagactgggacttgtttttaatgttattatttctattgaagagaattgtttgtcaaccggaaataaaaacattccattaagctcgggtatggcagtcactgcagaaataaagacaggtatgcgaagtgtaatcagttatcttcttagtcctttagaagagtcagtaacagaaagtttacgtgagcgttaagtttcagaagtccagtatttgctgctatacgtgctgcgtggcacttgccgtctgaacggcttgatccggaagccaagtcaaacaacagcgtgatgagcgtcagggcaaacaccaaggctctctcgatgacaccagaacaaattgaatacgtgctaggaaaagctaccgagt

(19) United States
(12) Patent Application Publication
(10) Pub. No.: US 2026/0232782 A1
(43) Pub. Date: Aug. 13, 2026

(54) HEMOLYSIN ANTIGENS AND VACCINE EMBODIMENTS FOR BACTERIAL INFECTION

(71) Applicant: Baylor College of Medicine, Houston, TX (US)

(72) Inventors: Yikun Xing, Houston, TX (US); Anthony Maresso, Houston, TX (US); Justin R. Clark, Houston, TX (US)

(73) Assignee: Baylor College of Medicine, Houston, TX (US)

(21) Appl. No.: 19/156,551

(22) PCT Filed: Feb. 16, 2024
(86) PCT No.: PCT/US2024/016249

§ 371 Date: Aug. 14, 2025

Related U.S. Application Data
(60) Provisional application No. 63/485,490, filed on Feb. 16, 2023.
(60) Provisional application No. 63/597,257, filed on Nov. 8, 2023.

(51) Int. Cl. A61K 39/108; A61K 39/00; A61P 37/04

(52) U.S. Cl. CPC A61K 39/0258; A61P 37/04; A61K 2039/545

(57) ABSTRACT

Embodiments of the disclosure include methods of treating, preventing, reducing the risk of, delaying the onset of, and/or reducing the severity of an infection (including pathogenic) in an individual infected with a bacteria from the Gammaproteobacteria Class. In specific embodiments, the methods comprise the step of administering to the individual an effective amount of an immunogenic composition comprising a non-acylated/inactive form alphahemolysin (HlyA) and/or a non-acylated/inactive form, or functional fragment(s) of either. In specific embodiments, the individual is also provided a composition comprising SinH or a functional fragment thereof.

### CLAIM OF PRIORITY

This application claims the benefit of U.S. Provisional Application No. 63/597,257 filed on Nov. 8, 2023 and U.S. Provisional Application No. 63/485,490 filed on Feb. 16, 2023, both of which are incorporated by reference herein.

### STATEMENT AS TO FEDERALLY SPONSORED RESEARCH

This invention was made with government support under AI157981 and AI144297 awarded by the National Institutes of Health. The government has certain rights in the invention.

### BACKGROUND

### I. Technical Field

This invention relates at least to the fields of microbiology, bacteriology, immunology, cell biology, molecular biology, and medicine.

### II. Background

Extraintestinal pathogenic E. coli (ExPEC) represents the most prevalent Gram-negative bacterial pathogen and is a primary contributor to mortality due to antimicrobial resistance (AMR) globally (both deaths attributable to and associated with AMR) [1,2]. ExPEC comprises the pathotypes of uropathogenic E. coli (UPEC), neonatal meningitis E. coli (NMEC), and septicemia-associated isolates (SEPEC) [3]. ExPEC is the primary cause of bacteremia and urinary tract infections (UTIs), and a frequent cause of neonatal meningitis [4,5]. In the United States, over 970,000 sepsis cases are admitted annually, with an 8.7% yearly increase in incidence among hospitalized patients, accounting for over 50% of hospital deaths [6,7]. Based on the Centers for Disease Control and Prevention (CDC) multiple cause-of-death data (1999-2014), 6% of all deaths involved sepsis, 22% of these cases listing sepsis as the underlying cause [8]. Moreover, in 2017, approximately 48.9 million new cases of sepsis were recorded globally, with 11 million sepsis-related deaths reported, accounting for 19.7% of all worldwide deaths [9]. In addition, sepsis management remains a major challenge for healthcare systems worldwide, resulting in a disproportionately high burden in terms of cost and hospital resource utilization. In the United States, sepsis management costs surpass those for any other disease, exceeding $24 billion in 2013, representing 13% of total hospital expenses and growing at three times the rate of other admissions [10]. E. coli has emerged as the predominant causative agent of bloodstream infections (BSIs) in both community and hospital settings over the past decade, accounting for 27.1% of all bacteremia cases. Moreover, the incidence rate of E. coli bacteremia is estimated at 48 per 100,000 person-years, exhibiting a notable increase with advancing age [11].

Next to their virulence, the second concerning feature of ExPEC is they are a leading cause of AMR, which frequently results in treatment failure, increased hospitalization rates, and exacerbated morbidity and mortality. A recent review assessing the global burden of bacterial AMR across 204 countries and territories identified antibiotic-resistant pathogenic E. coli as a primary cause of mortality associated with drug resistance, accounting for approximately 200,000 deaths due to antimicrobial-resistant E. coli and around 800,000 deaths linked to AMR E. coli in 2019 [2]. The CDC reports that over two million people in the United States contract antibiotic-resistant diseases annually, with AMR contributing an additional $20 billion to direct healthcare costs and roughly $35 billion in lost productivity each year [12]. Furthermore, other studies found that antimicrobial-resistant ExPEC infections impair the capacity of the immune system to clear infections, including complications in patients following prostate biopsy [13], solid organ transplant [14,15], or undergoing chemotherapy, dialysis, surgery, and joint replacement [12]. The recent emergence of a sequence type (ST) termed ST131, which combines both pan resistance with high levels of virulence, has become globally disseminated [16]. Furthermore, ST131 E. coli isolates maintain a balance between colonization, virulence, and antibiotic resistance without incurring a fitness cost, attributed to their distinct virulence profiles and expanded number of virulence genes compared to non-ST131 isolates [17,18]. In the United States, ST131 significantly contributes to the resistance of clinical E. coli isolates, accounting for approximately 70% of fluoroquinolone-resistant strains and over 50% of MDR isolates [19-21]. Other sequence types, such as ST95 and 73, also remain prevalent and well-recognized as highly pathogenic ExPEC strains among clinical isolates in patients with UTIs and bloodstream infections (BSIs) [22,23].

A vaccine against ExPEC is expected to reduce morbidity, mortality and help mitigate the AMR crisis. Over the past few decades, numerous groups have pursued protective immunity against ExPEC through various strategies, including the use of inactivated bacteria [24-27] or bacterial lysates [28-30], O-specific polysaccharide (O-antigen) conjugate vaccines [31-33], fimbrial-based vaccines such as FliC (or pilin) [34-36] and FimH (from type 1 fimbriae) [37], other fimbrial or non-fimbrial-based vaccines, such as adhesin FdeC [38], PapG fimbrial adhesin [39], and Dr fimbriae [40], proteins involved in nutrient uptake (IroN, lutA, IreA, FyuA, and siderophores) [41-49] and finally toxin-based products such as insoluble α-hemolysin or CNF1 (cytotoxic necrotizing factor 1) [50-52]. ExPEC4V (O1A, O2, O6A, and O25B) has elicited functional antibody responses and is currently in the phase 2 randomized controlled trial [53-58]. The high heterogeneity of O-specific polysaccharides may limit the development of a polysaccharide vaccine capable of preventing all ExPEC infections [54]. At the moment, no ExPEC vaccine has been approved by the U.S. Food and Drug Administration (FDA).

The α-hemolysin (HlyA) is a critical and commonly detected secreted cytotoxic virulence factor in ExPEC, associated with upper UTIs such as cystitis or pyelonephritis [59]. HlyA, a pore-forming toxin belonging to the RTX toxin family (repeats in toxin), exhibits cytotoxic activity against various species and cell types, potentially causing severe tissue damage [60]. Additionally, HlyA can lyse erythrocytes and damage effector immune cells at high concentrations [61,62], promote bladder epithelial cell exfoliation, and induce apoptosis in target host cells at low concentrations [63]. Clinically, HlyA is linked to severe UTIs that can lead to renal complications and permanent renal scarring [64,65], and may also cause endothelial damage and renal vasoconstriction [66]. The hlyA gene exhibits high prevalence in clinical E. coli isolates from patients with bloodstream infections [67-69], UTIs [70-72], and pregnant women, reaching up to 61.5% in E. coli strains causing cystitis and 78.6% in strains causing pyelonephritis [73]. Furthermore, an analysis of the hlyA gene distribution among major ExPEC clones revealed its prevalence was significantly higher in ST73 (64.6%) compared to ST131 (14.8%) and ST95 (13.5%) and was more frequently found in strains from phylogroup clades B and C [74,75].

Considering the significance of HlyA in ExPEC pathogenesis and the high prevalence of hlyA sequence in clinical E. coli isolates, pro-HlyA, the inactive precursor form of HlyA [76], as a vaccine candidate against ExPEC infections, with and without a previously reported autotransporter antigen showed to be highly protective [77]. Here, the present disclosure demonstrates that both pro-HlyA and a pro-HlyA/SinH combinatorial mixture generate robust protection against various virulent and concerning sequence types of ExPEC strains in multiple murine infection models.

### BRIEF SUMMARY

Embodiments of the disclosure include methods and compositions for the treatment, prevention, reducing the risk of, reduction in severity of one or more symptoms, and/or delay in onset of a bacterial infection, including a pathogenic bacterial infection. In specific embodiments, the compositions are immunogenic. In particular embodiments, the compositions are vaccines. The methods and compositions may be useful for treating, preventing, reducing the risk of, delaying the onset of, and/or reducing the severity of an infection in an individual of a bacteria from the Gammaproteobacteria Class. Any method may encompass administering to an individual in need thereof an effective amount of a composition comprising a non-acylated/inactive form alpha-hemolysin (HlyA) and/or a non-acylated/inactive form or functional fragment thereof.

Extraintestinal pathogenic E. coli (ExPEC) is a leading cause of worldwide morbidity and mortality, the top cause of antimicrobial resistant (AMR) infections, and the most frequent cause of life-threatening sepsis and urinary tract infections (UTI) in adults. The development of an effective and universal immunogenic composition, such as a vaccine, is complicated by this pathogen's pan-genome, its ability to mix and match virulence factors and AMR genes via horizontal gene transfer, an inability to decipher commensal from pathogens, and its intimate association and co-evolution with mammals. Using a pan virulome analysis of >20,000 sequenced E. coli strains, the secreted cytolysin HlyA was identified as a target for vaccine characterization. In specific embodiments, a catalytically inactive pure form of HlyA, expressed in an autologous host using its own secretion system, is highly immunogenic in a murine host, protects against several forms of ExPEC infection (including lethal bacteremia), and significantly lowers bacterial burdens in multiple organ systems. Interestingly, the combination of a previously reported autotransporter (SinH) with HlyA was exceedingly effective, inducing near complete protection against lethal challenge, including commonly used infection strains ST73 (CFT073) and ST95 (UTI89), as well as a mixture of ten of the most highly-virulent sequence types and strains from our clinical collection. Both HlyA and HlyA-SinH combinations also afforded some protection against UTI89 colonization in a murine UTI model. These findings indicate that recombinant, inactive hemolysin and/or its combination with SinH is useful as an E. coli immunogenic composition (e.g., a vaccine) against invasive disease.

Embodiments of the disclosure concern immunogenic compositions, such as vaccines for Extraintestinal Pathogenic E. coli (ExPEC) II. Specific embodiments concern efficacy of a toxin-autotransporter dual antigen approach.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1F show the ExPEC-associated genetical level of Hemolysin A, HlyA structure prediction, and the purification of pro-HlyA. An analysis of a database of 1,348 complete E. coli genomes that have been phylogenetically categorized shows that the hlyA sequence is predominantly found in ExPEC-associated sequence types of the B2 phylogroup. FIG. 1A is a phylogenetic representation of hlyA sequence distribution. BLAST was used to compare the hlyA nucleotide sequence to a database of complete E. coli genomes from NCBI's Genbank that had been sorted into phylogroups using a previously described in-house method and into sequence types using MLST software (https://github.com/tseemann/mlst). Pie charts were made using GraphPad Prism, and the final figure was created using Biorender. FIG. 1B shows amino acid alignment of HlyA. Tickmarks represent disagreements with the majority consensus at that residue and are colored using the Rasmol coloring scheme. Open reading frames overlapping with BLAST hits for hlyA were translated, aligned using Geneious Alignment (Geneious 2023.1.1), and then sorted using a tree annotation. The phylogenetic tree annotation was created using RXaML (version 8.1.1) and represents a consensus tree from 100 Bootstrap replicates with Vibrio parahaemolyticus hemolysin A (accession: WP_041955411.1) used as an outgroup. FIG. 1C is the overall predicted structure of HlyA. AlphaFold2 generated predicted structure shows three domains for HlyA with one linker: N-terminal adenylate cyclase domain (residues 1-279, top left structure), three helix bundle (residues 321-437, top middle structure), beta-helix C-terminal domain (residues 438-1023, top right structure), and linker between adenylate cyclase and helix-bundles (residues 280-320, grey). FIG. 1D is the predicted structure of C-terminal beta helix domain. A large beta helix dominates the overall organization of this domain. Both N- and C-ends of the domain contain two beta strands and two alpha helices. FIG. 1E is a SDS-page result for Plasmid pSU-hlyA (encoding the hlyA sequence) and plasmid pK184-hlyBD (encoding hlyB and hlyD sequence) which were co-transformed into E. coli BL21 (DE3) cells. Bacterial cultures expressing the recombinant pro-HlyA antigen secreted the protein into the supernatant, which was subsequently harvested, filtered, and concentrated. The purified antigen was analyzed by SDS-PAGE and stained with Coomassie blue stain buffer. Predicted size of pro-HlyA, 110 kDa. The SDS-PAGE result was annotated using BioRender. FIG. 1F is the coverage rate of pro-HlyA which was determined by per-band sequencing through mass spectrometry.

FIGS. 2A-2G evaluate the protective efficacy of pro-HlyA against UTI89 infections in the murine model of bacteremia and mortality. FIG. 2A is a scheme of the murine bacteremia model using UTI89. Female BALB/cJ mice were subcutaneously immunized with either pro-HlyA (N=16) or control supernatant (N=16), followed by an intraperitoneal (I.P.) injection of 1×10⁸ CFU of UTI89. After 16 hours of infection, organs (kidney, spleen, liver) were harvested, homogenized, and plated to determine bacterial loads (CFU/ml). FIG. 2B is a scatter plot with bar representing total UTI89 bacterial dissemination combining counts from all organs; or FIG. 2C the organ-specific UTI89 bacterial dissemination in each organ type post-necropsy. FIG. 2D is a the scheme of the murine mortality model using UTI89. Female BALB/cJ mice were subcutaneously immunized with pro-HlyA (N=12) or left unvaccinated (N=8), followed by an intraperitoneal (I.P.) injection of 5×10⁷ CFU of UTI89. Mice were monitored twice daily for 10 days. The moribund or deceased mice were euthanized and necropsied to determine bacterial levels in organs (CFU/ml). 4 surviving vaccinated mice were randomly chosen for euthanasia at 3 d.p.i, forming the 3 d.p.i group (N=4). All remaining survivors were euthanized at 10 d.p.i, forming the 10 d.p.i group (N=8). FIG. 2E is the survival rate of pro-HlyA immunized mice after UTI89 infection was assessed using the Gehan-Breslow-Wilcoxon comparison. FIG. 2F is a scatter plot with bar representing the total UTI89 bacterial dissemination combining counts from all organs at 3 d.p.i and 10 d.p.i; and FIG. 2G is the organ-specific UTI89 bacterial dissemination in each organ type (combining counts from 3 d.p.i and 10 d.p.i) post-necropsy. Schemes were created in BioRender. Scatter plots with bars and Kaplan Meier survival curves were exported from Graphpad Prism 9 and annotated using BioRender.

FIGS. 3A-3G evaluate the protective efficacy of Dual-Hit against UTI89 infections in the murine model of bacteremia and mortality. FIG. 3A is the scheme of the murine bacteremia model using UTI89. Female BALB/cJ mice were subcutaneously immunized with either Dual-Hit (N=15) or control supernatant (N=16), followed by an intraperitoneal (I.P.) injection of 1×10⁸ CFU of UTI89. After 16 hours post-infection, organs (kidney, spleen, liver) were harvested, homogenized, and plated to determine bacterial loads (CFU/ml). FIG. 3B is a scatter plot with bar representing total UTI89 bacterial dissemination combining counts from all organs; or FIG. 3C is the organ-specific UTI89 bacterial dissemination in each organ type post-necropsy. FIG. 3D is a scheme of the murine mortality model using UTI89. Female BALB/cJ mice were subcutaneously immunized with Dual-Hit (N=18) or left unvaccinated (N=7), followed by an intraperitoneal (I.P.) injection of 5×10⁷ CFU of UTI89. Mice were monitored twice daily for 10 days. The moribund or deceased mice were euthanized and necropsied to determine bacterial levels in organs (CFU/ml). 6 surviving vaccinated mice were randomly chosen for euthanasia at 3 d.p.i, forming the 3 d.p.i group (N=6). All remaining survivors were euthanized at 10 d.p.i, forming the 10 d.p.i group (N=12). (3E) The survival rate of Dual-Hit immunized mice after UTI89 infection was assessed using the Gehan-Breslow-Wilcoxon comparison. FIG. 3F is a scatter plot with bar representing the total UTI89 bacterial dissemination combining counts from all organs at 3 d.p.i and 10 d.p.i; and FIG. 3G is the organ-specific UTI89 bacterial dissemination in each organ type (combining counts from 3 d.p.i and 10 d.p.i) post-necropsy. Schemes were created in BioRender. Scatter plots with bars and Kaplan Meier survival curves were exported from Graphpad Prism 9 and annotated using BioRender.

FIGS. 4A-4D assess the protective efficacy of pro-HlyA and Dual-Hit against CFT073 infections in the murine model of mortality. FIG. 4A is a scheme of the murine mortality model using CFT073. Female BALB/cJ mice were subcutaneously immunized with pro-HlyA (N=12), Dual-Hit (N=12), or left unvaccinated (N=12), followed by an intraperitoneal (I.P.) injection of 1×10⁸ CFU of CFT073. Mice were monitored twice daily for 10 days. The moribund or deceased mice were euthanized and necropsied to determine bacterial levels in organs (kidney, spleen, liver, CFU/ml). Vaccinated mice that died of infection within 2 d.p.i formed the 2 d.p.i group for each vaccinated cohort (pro-HlyA, N=9; Dual-Hit, N=7). All remaining survivors were euthanized at 10 d.p.i, forming the 10 d.p.i group for each vaccinated cohort (pro-HlyA, N=3; Dual-Hit, N=5). FIG. 4B is survival rates of pro-HlyA or Dual-Hit immunized mice following CFT073 infection were analyzed using the Gehan-Breslow-Wilcoxon comparison. FIG. 4C is a scatter plot with bar representing the total CFT073 bacterial dissemination combining counts from all organs at 2 d.p.i and 10 d.p.i; and FIG. 4D is the organ-specific CFT073 bacterial dissemination in each organ type (combining counts from 2 d.p.i and 10 d.p.i) post-necropsy. Schemes were created in BioRender. Scatter plots with bars and Kaplan Meier survival curves were exported from Graphpad Prism 9 and annotated using BioRender.

FIGS. 5A-5C evaluate the protective efficacy of pro-HlyA and Dual-Hit against UTI89 or CFT073 in the murine model of UTI. FIG. 5A is a scheme of the murine UTI model using UTI89 or CFT073. Female BALB/cJ mice were subcutaneously immunized with either pro-HlyA (N=12/N=12), Dual-Hit (N=12/N=12) or remained unvaccinated (N=8/N=12), followed by a transurethral injection of 1×10⁸ CFU of UTI89 or CFT073. After 72 hours post-infection, bladders were harvested, homogenized, and plated to determine bacterial loads (CFU/ml). FIG. 5B is a scatter plot with bar representing bladder UTI89 bacterial dissemination; and FIG. 5C is a scatter plot with bar representing bladder CFT073 bacterial dissemination.

FIGS. 6A-6D evaluate the protective efficacy of Dual-Hit against a mixture of ten typical ExPEC strains infection in the murine model of mortality. FIG. 6A is a scheme of the murine mortality model using a mixture of ten ExPEC strains. Female BALB/cJ mice were subcutaneously immunized with Dual-Hit (N=22), or left unvaccinated (N=24), followed by an intraperitoneal (I.P.) injection of 1×10⁸ CFU (in total) of a mixture of ten typical ExPEC strains. Mice were monitored twice daily for 10 days. The moribund or deceased mice were euthanized and necropsied to determine bacterial levels in organs (kidney, spleen, liver, CFU/ml). Vaccinated mice that died of infection within 1 d.p.i (N=2) and four randomly selected surviving vaccinated mice euthanized at 3 d.p.i (N=4) formed the 3 d.p.i group. All remaining surviving mice were euthanized at 10 d.p.i, forming the 10 d.p.i group (N=16). FIG. 6B are survival rates of Dual-Hit immunized mice following a mixture of ten typical ExPEC strains infection were analyzed using the Gehan-Breslow-Wilcoxon comparison. FIG. 6C is a scatter plot with bar representing the total bacterial dissemination of mixture of ten typical ExPEC strains combining counts from all organs at 3 d.p.i and 10 d.p.i; and FIG. 6D is the organ-specific bacterial dissemination of mixture of ten typical ExPEC strains in each organ type (combining counts from 3 d.p.i and 10 d.p.i) post-necropsy. Schemes were created in BioRender. Scatter plots with bars and Kaplan Meier survival curves were exported from Graphpad Prism 9 and annotated using BioRender.

FIGS. 7A-7D evaluate the protective efficacy of pro-HlyA antigen alone against the mixture of ST131 ExPEC strains infection in the murine model of mortality. It was evaluated whether pro-HlyA alone could provide sufficient cross-reactive protection against a mixture of five ST131 ExPEC strains that lacked the hlyA gene (including ST131-H30R lineage) in the murine model of mortality. FIG. 7A include the experimental group mice (N=16) which received three subcutaneous immunizations with pro-HlyA with an alum adjuvant at a 2:1 ratio (antigen/alum) on days 0, 14, and 28, while the control group (N=16) mice were injected with equal volumes of control supernatant and alum adjuvant. On day 42, all mice were intraperitoneally challenged with a mixture of five ST131 ExPEC strains (1×10⁸ CFU/mouse in total, JJ1886, JJ1901, JJ2050, JJ2528, JJ2547, in equal proportions). Mice were closely monitored over the next 10 days for morbidity and mortality twice daily. Survival data were collected over time, and moribund or deceased mice were euthanized and necropsied to determine the bacterial loads in kidneys, spleen, and liver. Harvested organs were homogenized, and bacterial burdens within infected tissues were quantified by determining CFU (CFU/ml). FIG. 7B illustrates the findings demonstrate that mice in the control group died within 1 d.p.i. Among the vaccinated mice during the 10-day observation period, 11 out of 16 pro-HlyA vaccinated mice died within 1 d.p.i (represent the 1 d.p.i group). Two mice died at 6 d.p.i and 9 d.p.i, respectively, resulting in a survival rate of 18.8% at 10 d.p.i (Adjusted P-value, P=0.0166) (All the remaining vaccinated mice were euthanized at 10 d.p.i (N=3), together with the mice that died at 6 d.p.i and 9 d.p.i (N=2), forming the 10 d.p.i group (N=5)). FIG. 7C show all the remaining vaccinated mice were euthanized at 10 d.p.i (N=3), together with the mice that died at 6 d.p.i and 9 d.p.i (N=2), forming the 10 d.p.i group (N=5). When combining counts from all organs, pro-HlyA immunized mice exhibited a statistically significant reduction in bacterial burden at 1 d.p.i (Adjusted P-value, P=0.0173) and 10 d.p.i (Adjusted P-value, P<0.0001) compared to the control group mice. FIG. 7D shows that upon combining bacterial burden counts at both 1 d.p.i and 10 d.p.i and analyzing them by each organ, pro-HlyA immunized mice only demonstrated statistically significant decreases in bacterial burdens in the spleen (Adjusted P-value, P=0.0024) and liver (Adjusted P-value, P=0.0012) compared to the control group mice. Schemes were created in BioRender. Scatter plots with bars and Kaplan Meier survival curves were exported from Graphpad Prism 9 and annotated using BioRender. Collectively, these findings indicate that in some embodiments pro-HlyA is only protective against ExPEC that encodes the hemolysin and will not provide protection against ST131 that is likely virulent but does not require HlyA for its virulence. Whereas such a finding may demonstrate the specificity of the results observed in FIG. 2 towards HlyA, it also means that strains that lack this antigen may still cause substantial disease even if the recipient is vaccinated.

FIGS. 8A-8D evaluate the protective efficacy of Dual-Hit mRNA vaccine against UTI89 infection in the murine model of mortality. With the advancement of novel vaccine technology in recent years, it is useful to develop an innovative mRNA vaccine targeting against ExPEC. The mRNA vaccine encoded both hlyA and sinH-3 sequences, incorporating an IL-2 signal peptide and a P2A self-cleavage sequence, built as the Dual-Hit mRNA construct, which is then encapsulated in cationic lipid nanoparticles. FIG. 8A is a scheme of the murine mortality model using UTI89. Male BALB/cJ mice were intramuscular (I.M.) immunized with Dual-Hit mRNA vaccine (low-dose, N=10; high-dose, N=7), or Tris-buffer (N=12), followed by an intraperitoneal (I.P.) injection of 1×10⁸ CFU (in total) of UTI89. Mice were monitored twice daily for 10 days. The moribund or deceased mice were euthanized and necropsied to determine bacterial levels in organs (kidney, spleen, liver, CFU/ml). FIG. 8B shows survival rates of Dual-Hit mRNA vaccine immunized mice following UTI89 infection were analyzed using the Gehan-Breslow-Wilcoxon comparison. The results indicated that both control and low-dose group mice died within 1 d.p.i, while 1 out of 7 high-dose vaccinated mice survived during the 10-day observation period. Furthermore, although one high-dose vaccinated mouse survived at 10 d.p.i, there is no statistical increase of both vaccinated groups in survival rate post-UTI89 infection (High-dose group, Adjusted P-value, P=0.1904), inferring the Dual-Hit mRNA vaccine only provided limited protection against UTI89 bacteriemia in murine mortality model. FIG. 8C is a scatter plot with bar representing the total bacterial dissemination of UTI89 combining counts from all organs. Regarding bacterial burden measurements, combining counts from all organs, mice immunized with either low-dose (Adjusted P-value, P<0.0001) or high-dose (Adjusted P-value, P=0.0043) Dual-Hit mRNA vaccine had a statistically significant reduction in bacterial burdens compared to control mice. However, this level of protection was insufficient to increase the survival rate after UTI89 infection. FIG. 8D is the organ-specific bacterial dissemination of UTI89 in each organ type post-necropsy. No discernable differences in organ-specific bacterial dissemination were observed among the three groups. Schemes were created in BioRender. Scatter plots with bars and Kaplan Meier survival curves were exported from Graphpad Prism 9 and annotated using BioRender.

### DETAILED DESCRIPTION

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

The term “functional” as used herein refers to a modified biological component that functions according to its purpose. For example, a fragment of HlyA that is able to function as an antigen, is a functional fragment of HlyA for the purposes of this disclosure.

It is contemplated that any embodiment discussed in this specification can be implemented with respect to any method or composition of the invention, and vice versa. Furthermore, compositions of the invention can be used to achieve methods of the invention.

### I. General Embodiments

Embodiments of the disclosure concern the secreted cytolysin α-hemolysin (HlyA) as an immunogenic composition, such as a vaccine. It is demonstrated herein that in specific embodiments the inactive pure form of HlyA is highly immunogenic in a mammalian host, protects against several forms of ExPEC infection (including lethal bacteremia), and significantly lowers bacterial burdens in multiple organ systems. In specific embodiments, the combination of an autotransporter (SinH) with HlyA was exceedingly effective, inducing near complete protection against lethal challenge, including commonly-used infection strains ST73 (CFT073) and ST95 (UTI89), as well as a mixture of ten of the most highly-virulent sequence types and strains. Both HlyA and HlyA-SinH combinations also afforded some protection against UTI89 colonization in a murine urinary tract infection (UTI) model. These findings indicate that recombinant, inactive hemolysin and/or its combination with SinH are useful for the development of an bacterial vaccine against invasive disease, including for any type of bacteria from the Gammaproteobacteria Class, such as at least E. coli.

Embodiments of the disclosure include methods of treating, preventing, reducing the risk of, delaying the onset of, and/or reducing the severity of an infection (including pathogenic) in an individual infected with a bacteria from the Gammaproteobacteria Class. In specific embodiments, the methods comprise the step of administering to the individual an effective amount of a composition comprising a non-acylated/inactive form alpha-hemolysin (HlyA) and/or a non-acylated/inactive form, or functional fragment(s) of either. In specific embodiments, the inactive HlyA is from the absence of the catalytic acylation enzyme, HlyC. The inactive HlyA may be non-acylated. In some cases, the inactive HlyA may not be capable of inducing pore formation and/or may not be capable of inducing calcium influx. In specific embodiments, the bacteria is in the Enterobacterales Order and may be in the Enterobacteriaceae family. In embodiments, the HlyA composition comprises a fragment of HlyA. In embodiments, the fragment of HlyA is pro-HlyA. In embodiments, the fragment of HlyA comprises 99%, 98%, 97%, 96%, 95%, 94%, 93%, 92%, 91%, 90%, 95%, 90%, 85%, 80%, 75%, 70%, 65%, 60%, 55%, 50%, 45%, 40%, 35%, 30% or greater sequence similarity to SEQ ID NO: 10.

For any method or composition embodiment herein, the bacteria may be in the Genus Escherichia, Vibrio Shigella, Salmonella, Yersinia, Enterobacter, Morganella, or Citerobacter. Specific examples include at least Escherichia coli, Vibrio parahaemolyticus, Shigella sonnei, Shigella flexneri, Shigella boydii, Shigella dysenteriae, Salmonella bongori, Salmonella enterica, Yersinia pestis, Yersinia enterocolitica, Yersinia pseudotuberculosis, Enterobacter huaxiensis, Enterobacter chuandaensis, Enterobacter aerogenes, Enterobacter amnigenus, Enterobacter arachidis, Enterobacter asburiae, Enterobacter carcinogenus, Enterobacter cloacae, Enterobacter cowanii, Enterobacter dissolvans, Enterobacter gergoviae, Enterobacter helveticus, Enterobacter hormaechei, Enterobacter kobei, Enterobacter ludwigii, Enterobacter mori, Enterobacter nimipressuralis, Enterobacter oryzae, Enterobacter pulveris, Enterobacter pyrinus, Enterobacter radicincitans, Enterobacter soli, Enterobacter taylorae, Enterobacter turicensis, Morganella morganii, Citerobacter freundii, Citerobacter koseri, Citerobacter amalonaticus, Citerobacter farmeri, Citerobacter youngae, Citerobacter braakii, Citerobacter werkmanii, Citerobacter sedlakii, Citerobacter rodentium, Citerobacter gillenii, or Citerobacter murliniae.

For any method or composition embodiment herein, the HlyA is from a bacteria member of the Enterobacterales Order, including of the Enterobacteriaceae family. The HlyA may be from a bacteria member of the Genus Escherichia, Vibrio Shigella, Salmonella, Yersinia, Enterobacter, Morganella, or Citerobacter. The HlyA may be from Escherichia coli, Vibrio parahaemolyticus, Shigella sonnei, Shigella flexneri, Shigella boydii, Shigella dysenteriae, Salmonella bongori, Salmonella enterica, Yersinia pestis, Yersinia enterocolitica, Yersinia pseudotuberculosis, Enterobacter huaxiensis, Enterobacter chuandaensis, Enterobacter aerogenes, Enterobacter amnigenus, Enterobacter arachidis, Enterobacter asburiae, Enterobacter carcinogenus, Enterobacter cloacae, Enterobacter cowanii, Enterobacter dissolvans, Enterobacter gergoviae, Enterobacter helveticus, Enterobacter hormaechei, Enterobacter kobei, Enterobacter ludwigii, Enterobacter mori, Enterobacter nimipressuralis, Enterobacter oryzae, Enterobacter pulveris, Enterobacter pyrinus, Enterobacter radicincitans, Enterobacter soli, Enterobacter taylorae, Enterobacter turicensis, Morganella morganii, Citerobacter freundii, Citerobacter koseri, Citerobacter amalonaticus, Citerobacter farmeri, Citerobacter youngae, Citerobacter braakii, Citerobacter werkmanii, Citerobacter sedlakii, Citerobacter rodentium, Citerobacter gillenii, or Citerobacter murliniae, in some cases.

Embodiments of the disclosure include methods of providing an immunogenic composition to an individual in need thereof. Embodiments of the disclosure include methods of vaccinating an individual for a pathogenic infection of a bacteria from the Gammaproteobacteria Class, comprising the step of administering to the individual an effective amount of a composition comprising a non-acylated/inactive form of alpha-hemolysin (HlyA) and/or a non-acylated/inactive form or functional fragment thereof. The bacteria may be in the Enterobacterales Order, including in the Enterobacteriaceae family.

Embodiments of the disclosure include methods of preventing, reducing the risk of, delaying the onset of, and/or reducing the severity of the systemic spread of bacteria to one or more major organ systems of an individual, comprising the step of administering to the individual an effective amount of a composition comprising the non-acylated/inactive form of HlyA and/or a non-acylated/inactive form or functional fragment thereof or a functional fragment thereof.

Embodiments of the disclosure include methods of reducing the bacterial burden of an individual, comprising the step of administering to the individual an effective amount of a composition comprising the non-acylated/inactive form of HlyA and/or a non-acylated/inactive form or functional fragment thereof

Embodiments of the disclosure include methods of preventing, reducing the risk of, delaying the onset of, and/or reducing the severity of bacteremia in an individual, comprising the step of administering to the individual an effective amount of a composition comprising the non-acylated/inactive form of HlyA and/or a non-acylated/inactive form or functional fragment thereof.

Embodiments of the disclosure include methods of preventing, reducing the risk of, delaying the onset of, and/or reducing the severity of a urinary tract infection in an individual, comprising the step of administering to the individual an effective amount of a composition comprising the non-acylated/inactive form of HlyA and/or a non-acylated/inactive form or functional fragment thereof.

Embodiments of the disclosure include methods of preventing, reducing the risk of, delaying the onset of, and/or reducing the severity of colonization of bacteria from the Gammaproteobacteria Class in an individual, comprising the step of administering to the individual an effective amount of a composition comprising the non-acylated/inactive form of HlyA and/or a non-acylated/inactive form or functional fragment thereof.

Embodiments of the disclosure include methods of preventing, reducing the risk of, delaying the onset of, and/or reducing the severity of sepsis in an individual, comprising the step of administering to the individual an effective amount of a composition comprising the non-acylated/inactive form of HlyA and/or a non-acylated/inactive form or functional fragment thereof.

Embodiments of the disclosure include any method that further comprises the step of administering to the individual an effective amount of a composition comprising SinH or a functional fragment thereof. In some embodiments, the composition comprises the inactive HlyA or a functional fragment thereof and is provided to the individual in the same formulation as the composition comprising the SinH or a functional fragment thereof. In certain embodiments, the composition comprises the inactive HlyA or a functional fragment thereof and is provided to the individual in a different formulation as the composition comprising the SinH or a functional fragment thereof. The composition may comprise the inactive HlyA or a functional fragment thereof, and it may be administered to the individual prior to, during, and/or subsequent to administering the composition comprising the SinH or a functional fragment thereof. In embodiments, the fragment of SinH comprises 99%, 98%, 97%, 96%, 95%, 94%, 93%, 92%, 91%, 90%, 95%, 90%, 85%, 80%, 75%, 70%, 65%, 60%, 55%, 50%, 45%, 40%, 35%, 30% or greater sequence similarity to SEQ ID NO: 10.

In particular embodiments of any method or composition herein, the bacteria may be drug-resistant, including multidrug-resistant. The bacteria may be E. coli and the E. coli may be ST73, ST95, or ST131. In specific embodiments, the bacteria is extraintestinal pathogenic Escherichia coli (ExPEC).

In embodiments in which a functional fragment of SinH is utilized it may comprise extracellular domain 1, domain 2, and/or domain 3.

In specific embodiments, the infection may be local, or thought it may be systemic. When localized, it may be on the skin, in the brain, in the mouth, in the throat, in the nose, in the eye, in the ear, in the esophagus, in the stomach, in the small intestine, in the large intestine, in the bladder, in the urinary tract, in the spleen, in the uterus, in the cervix, in the testes, in the rectum, in the anus, in the heart, or a combination thereof, and so forth. The individual may or may not be immunocompromised. The individual may be an infant, child, adolescent, or adult. The individual may be at least 10, 20, 30, 40, 50, 60, 65, 70, 75, 80, 85, 90, 95, or 100 or more years of age. The individual may or may not have recurrent urinary tract infections. The individual may be in a medical facility (e.g., hospital or nursing home or skilled nursing home or long-term care facility) or may have been or will be in a medical facility within 1, 2, 3, 4, 5, 6, or 7 days, or within 1, 2, 3, 4 week, or within 1, 2, 3, 4, 5, 6, 7, 8, 9, 10, 11, or 12 months, or within 1, 2, 3, 4, 5, 6, 7, 8, 9, 10, or more years, as examples. The individual may lack or have a pathogenic E. coli infection.

In any method encompassed herein, the administering step occurs once, although it may occur more than once, such as having a duration between successive administrations being, or being at least, or being no more than within 1, 2, 3, 4, 5, 6, or 7 days, or within 1, 2, 3, 4 week, or within 1, 2, 3, 4, 5, 6, 7, 8, 9, 10, 11, or 12 months, or within 1, 2, 3, 4, 5, 6, 7, 8, 9, 10, or more years.

In specific embodiments, the composition comprises an adjuvant. In specific embodiments in which SinH is utilized, the SinH functional fragment is part of a conjugate, although the inactive HlyA functional fragment may be part of a conjugate, or both may be part of a conjugate.

Embodiments of the composition can comprise a non-acylated/inactive form alpha-hemolysin (HlyA) and/or a non-acylated/inactive form or functional fragment thereof in a pharmaceutically acceptable excipient.

Any method may further comprise an antibiotic, SinH, the extracellular domain 1 of SinH, the extracellular domain 2 of SinH, the extracellular domain 3 of SinH, one or more O-antigens, one or more K-antigens, and/or FimH (Type 1 fimbriae protein).

Embodiments of the disclosure include kits comprising any composition encompassed herein, housed in a suitable container.

### II. Pharmaceutical Preparations

Any of the HylA and/or SinH compositions of the disclosure may be in a pharmaceutical composition. Pharmaceutical compositions of the present disclosure comprise an effective amount of one or more HylA and/or SinH compositions of any kind dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that comprises at least one HylA and/or SinH compositions will be known to those of skill in the art in light of the present disclosure, as exemplified by Remington: The Science and Practice of Pharmacy, 21^{st} Ed. Lippincott Williams and Wilkins, 2005, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The HylA and/or SinH compositions may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The HylA and/or SinH compositions may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present disclosure, the composition of the present disclosure suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present disclosure, the composition is combined with the carrier in any convenient and practical manner, i.e., by solution, suspension, emulsification, admixture, encapsulation, absorption and the like. Such procedures are routine for those skilled in the art.

In a specific embodiment of the present disclosure, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present disclosure may concern the use of a pharmaceutical lipid vehicle compositions that include HylA and/or SinH compositions, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the HylA and/or SinH compositions may be dispersed in a solution comprising a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present disclosure administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

### A. Alimentary Compositions and Formulations

In preferred embodiments of the present invention, the HylA and/or SinH compositions are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

### B. Parenteral Compositions and Formulations

In further embodiments, HylA and/or SinH compositions may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

### C. Miscellaneous Pharmaceutical Compositions and Formulations

In other preferred embodiments of the invention, the active compound HylA and/or SinH compositions may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

I. Listing of Sequences

| Name | Sequence |
|---|---|
| SinH | mlrwkrciml tfisgaafaa peinvkqnes lpdlgsqaaq qdeqtnkgks lkergadyvi nsatqgfenl |
| GenBAnk | tpealesqar sylqsqitst aqsyiedtls pygkvrsnls igqggdldgs sidyfvpwyd nqttvyfsqf |
| Accession | saqrkedrti gniglgvrhn fdkwllggni fydydftrgh rrlgigaeaw tdylkfsgny yhplsdwkds |
| No. | edfdfyeerp asgwdiraea wlpaypalgg kivfehyygd evalfgtdnl ekdpfavtlg vkyqpvplia |
| AMQ52229 | vgtdfkagtg dntdttvnat Inyqfgvplk dqldpdkvsa ahslmgsrhd fvernnfivl eykekdpldv |
| (intimin- | tlwlkadamn ehpecvikdt peeaiglekc kwtinalvnh hykivaaswq aknnaartlv mpvikentlt |
| like | egnnnhwnlv lpvwqyssdk aeqgklntwr vrlalkdekg nrqnsgvvei tvqqdrkiel ivnnianvpe |
| protein | ennhsheasa qadgvdgvvm dlditdsfgd ntdrngnvlp qdnlnpqlfd andkkvtltn npcttetpcv |
| SinH | fiakqdkekg tvtlsstlpg tfrwkakaap yddsnyvdvt flgadiggln afiyrvgaak svnlignkep |
| [Escherichia | 1plnsryrfv lwrdankdgv fqlsekltde emkqydyqwe ftghsvngnt gaqanttnvd ieipatnkea |
| coli | aakfsaqvtd gvqgyglqvn yskt (SEQ ID NO: 1) |
| JJ1887]) | |
| SinH | GTCATCAACTCCGCCACGCAAGGGTTTGAAAACTTGACCCC |
| Domain | TGAGGCGCTGGAATCTCAGGCCAGAAGCTATCTGCAAAGTCAAATCA |
| β- | CCTCAACCGCACAATCTTATATTGAAGACACACTCTCTCCCTACGGTA |
| barrel_ | AGGTCCGTTCGAACCTCTCCATTGGTCAGGGCGGCGATCTGGATGGCA |
| domain | GTTCCATCGATTATTTTGTTCCCTGGTACGATAATCAAACCACTGTTTA |
| (residues_ | TTTCAGCCAATTTTCTGCGCAACGAAAAGAAGATCGTACGATCGGAAA |
| 59-336) | TATAGGGCTTGGTGTAAGACATAATTTTGATAAATGGTTATTGGGTGG |
| | AAATATATTTTATGATTATGATTTTACCCGTGGACATCGCCGTTTAGGT |
| | ATAGGCGCTGAAGCGTGGACTGATTATTTAAAATTTTCTGGTAACTAT |
| | TATCATCCGCTTTCTGACTGGAAAGACTCTGAAGATTTCGACTTTTATG |
| | AAGAACGCCCTGCGAGCGGTTGGGATATTCGTGCCGAAGCCTGGCTTC |
| | CCGCTTATCCGCAGCTTGGCGGTAAAATCGTTTTCGAACATTATTACG |
| | GGGATGAAGTTGCGCTATTTGGTACCGACAATCTAGAGAAAGATCCCT |
| | TCGCTGTCACGTTAGGCGTGAAATACCAGCCTGTTCCGCTCATTGCTG |
| | TAGGAACGGACTTCAAAGCAGGAACCGGTGATAACACCGATACCACG |
| | GTCAACGCAACGCTTAACTACCAGTTTGGCGTCCCGCTCAAAGACCAG |
| | CTTGATCCTGACAAAGTGAGCGCGGCTCACTCACTCATGGGCAGCCGT |
| | CACGACTTTGTTGAACGTAACAAC (SEQ ID NO: 2) |
| SinH | GTCACTTTGTGGCTGAAAGCTGATGCGATGAATGAACATCC |
| Domain 1 | CGAGTGCGTGATTAAAGATACGCCAGAAGAAGCTATCGGGCTGGAAA |
| (residues_ | AATGTAAATGGACCATTAACGCGCTTGTCAACCACCACTACAAAATTG |
| 350-472) | TCGCTGCGTCATGGCAGGCGAAAAACAATGCCGCCCGCACGCTGGTG |
| | ATGCCGGTTATCAAAGAGAATACCCTGACAGAGGGCAACAATAACCA |
| | CTGGAACCTGGTGCTGCCCGTCTGGCAGTACAGCTCTGACAAAGCCGA |
| | ACAAGGAAAACTCAATACCTGGCGGGTTCGCCTGGCGCTGAAAGATG |
| | AAAAGGGCAACCGACAGAACTCAGGCGTGGTGGAAATCACCGTT |
| | (SEQ ID NO: 3) |
| SinH | AGTGCACAGGCTGATGGTGTAGATGGTGTGGTGATGGATCTCGATATT |
| Domain 2 | ACTGATAGCTTTGGCGACAACACTGACCGCAACGGTAATGTCTTGCCG |
| (residues_ | CAGGATAATCTCAACCCGCAGCTGTTTGATGCCAACGATAAAAAAGT |
| 499-601) | GACGCTGACGAATAACCCCTGCACTACCGAGACTCCCTGTGTCTTTAT |
| | CGCCAAACAAGATAAAGAAAAGGGGACCGTTACGCTCTCCAGTACGT |
| | TGCCTGGCACCTTCCGTTGGAAGGCGAAAGCTGCCCCCTATGATGACA |
| | GTAACTATGTTGACGTAACGTTC (SEQ ID NO: 4) |
| SinH | GGCGGGCTGAATGCCTTTATCTATCGCGTTGGTGCGGCTAAATCCGTG |
| Domain 3 | AATCTTATCGGCAATAAAGAACCGTTGCCCCTCAATAGCCGATATCGC |
| residues | TTTGTGTTGTGGCGCGACGCTAACAAAGACGGTGTGTTCCAGCTATCT |
| 607-725 | GAAAAGCTCACTGACGAAGAGATGAAACAGTATGACTACCAATGGGA |
| | ATTCACGGGGCATAGCGTAAATGGCAATACTGGCGCGCAAGCCAATA |
| | CCACTAACGTCGATATCGAGATCCCGGCAACGAACAAGGAAGCGGCA |
| | GCGAAGTTTAGTGCCCAGGTAACCGATGGTGTGCAAGGGTACGGTTTG |
| | CAGGTCAATTACAGCAAGACATAG (SEQ ID NO: 5) |
| Full | ATGCTGCGATGGAAACGCTGTATTATGCTAACATTTATCTCT |
| Length | GGTGCTGCTTTCGCGGCACCAGAGATAAATGTTAAGCAAAACGAATC |
| Protein | GTTACCTGATTTAGGTAGCCAGGCAGCACAACAGGATGAACAAACCA |
| from | ACAAGGGTAAATCGCTGAAAGAGCGCGGAGCCGATTACGTCATCAAC |
| JJ1887 | TCCGCCACGCAAGGGTTTGAAAACTTGACCCCTGAGGCGCTGGAATCT |
| | CAGGCCAGAAGCTATCTGCAAAGTCAAATCACCTCAACCGCACAATCT |
| | TATATTGAAGACACACTCTCTCCCTACGGTAAGGTCCGTTCGAACCTC |
| | TCCATTGGTCAGGGCGGCGATCTGGATGGCAGTTCCATCGATTATTTT |
| | GTTCCCTGGTACGATAATCAAACCACTGTTTATTTCAGCCAATTTTCT |
| | GCGCAACGAAAAGAAGATCGTACGATCGGAAATATAGGGCTTGGTGTA |
| | AGACATAATTTTGATAAATGGTTATTGGGTGGAAATATATTTTATGAT |
| | TATGATTTTACCCGTGGACATCGCCGTTTAGGTATAGGCGCTGAAGCG |
| | TGGACTGATTATTTAAAATTTTCTGGTAACTATTATCATCCGCTTTCTG |
| | ACTGGAAAGACTCTGAAGATTTCGACTTTTATGAAGAACGCCCTGCGA |
| | GCGGTTGGGATATTCGTGCCGAAGCCTGGCTTCCCGCTTATCCGCAGC |
| | TTGGCGGTAAAATCGTTTTCGAACATTATTACGGGGATGAAGTTGCGC |
| | TATTTGGTACCGACAATCTAGAGAAAGATCCCTTCGCTGTCACGTTAG |
| | GCGTGAAATACCAGCCTGTTCCGCTCATTGCTGTAGGAACGGACTTCA |
| | AAGCAGGAACCGGTGATAACACCGATACCACGGTCAACGCAACGCTT |
| | AACTACCAGTTTGGCGTCCCGCTCAAAGACCAGCTTGATCCTGACAAA |
| | GTGAGCGCGGCTCACTCACTCATGGGCAGCCGTCACGACTTTGTTGAA |
| | CGTAACAACTTTATTGTTCTGGAATATAAAGAGAAAGATCCCCTCGAT |
| | GTCACTTTGTGGCTGAAAGCTGATGCGATGAATGAACATCCCGAGTGC |
| | GTGATTAAAGATACGCCAGAAGAAGCTATCGGGCTGGAAAAATGTAA |
| | ATGGACCATTAACGCGCTTGTCAACCACCACTACAAAATTGTCGCTGC |
| | GTCATGGCAGGCGAAAAACAATGCCGCCCGCACGCTGGTGATGCCGG |
| | TTATCAAAGAGAATACCCTGACAGAGGGCAACAATAACCACTGGAAC |
| | CTGGTGCTGCCCGTCTGGCAGTACAGCTCTGACAAAGCCGAACAAGG |
| | AAAACTCAATACCTGGCGGGTTCGCCTGGCGCTGAAAGATGAAAAGG |
| | GCAACCGACAGAACTCAGGCGTGGTGGAAATCACCGTTCAGCAGGAC |
| | CGTAAAATTGAGCTGATTGTTAATAACATCGCGAACGTGCCGGAAGA |
| | GAACAACCACAGCCACGAAGCCAGTGCACAGGCTGATGGTGTAGATG |
| | GTGTGGTGATGGATCTCGATATTACTGATAGCTTTGGCGACAACACTG |
| | ACCGCAACGGTAATGTCTTGCCGCAGGATAATCTCAACCCGCAGCTGT |
| | TTGATGCCAACGATAAAAAAGTGACGCTGACGAATAACCCCTGCACT |
| | ACCGAGACTCCCTGTGTCTTTATCGCCAAACAAGATAAAGAAAAGGG |
| | GACCGTTACGCTCTCCAGTACGTTGCCTGGCACCTTCCGTTGGAAGGC |
| | GAAAGCTGCCCCCTATGATGACAGTAACTATGTTGACGTAACGTTCCT |
| | GGGGGCTGACATCGGCGGGCTGAATGCCTTTATCTATCGCGTTGGTGC |
| | GGCTAAATCCGTGAATCTTATCGGCAATAAAGAACCGTTGCCCCTCAA |
| | TAGCCGATATCGCTTTGTGTTGTGGCGCGACGCTAACAAAGACGGTGT |
| | GTTCCAGCTATCTGAAAAGCTCACTGACGAAGAGATGAAACAGTATG |
| | ACTACCAATGGGAATTCACGGGGCATAGCGTAAATGGCAATACTGGC |
| | GCGCAAGCCAATACCACTAACGTCGATATCGAGATCCCGGCAACGAA |
| | CAAGGAAGCGGCAGCGAAGTTTAGTGCCCAGGTAACCGATGGTGTGC |
| | AAGGGTACGGTTTGCAGGTCAATTACAGCAAGACATAG (SEQ ID NO: 6) |
| SinH- | FIVLEYKEKDPLDVTLWLKADAMNEHPECVIKDTPEEAIGLEKCK |
| domain-1 | WTINALVNHHYKIVAASWQAKNNAARTLVMPVIKENTLTEGNNNHW |
| | NLVLPVWQYSSDKAEQGKLNTWRVRLALKDEKGNRQNSGVVEITV |
| | (SEQ ID NO: 7) |
| SinH- | QQDRKIELIVNNIANVPEENNHSHEASAQADGVDGVVMDLDITDSFGDN |
| domain-2 | TDRNGNVLPQDNLNPQLFDANDKKVTLTNNPCTTETPCVFIAKQDKEKG |
| | TVTLSSTLPGTFRWKAKAAPYDDSNYVDVTF (SEQ ID NO: 8) |
| SinH- | LGADIGGLNAFIYRVGAAKSVNLIGNKEPLPLNSRYRFVLWRDANKDGV |
| domain-3 | FQLSEKLTDEEMKQYDYQWEFTGHSVNGNTGAQANTTNVDIEIPATNKE |
| | AAAKFSAQVTDGVQGYGLQVNYSKT (SEQ ID NO: 9) |
| Pro- | MTTITTAQIKSTLQSAKQSAANKLHSAGQSTKDALKKAAEQTRNAGNRLI |
| HlyA | LLIPKDYKGQGSSLNDLVRTADELGIEVQYDEKNGTAITKQVFGTAEKLI |
| e. coli | GLTERGVTIFAPQLDKLLQKYQKAGNILGGGAENIGDNLGKAGGILSTFQ |
| protein | NFLGTALSSMKIDELIKKQKSGGNVSSSELAKASIELINQLVDTVASLNNN |
| sequence | VNSFSQQLNTLGSVLSNTKHLNGVGNKLQNLPNLDNIGAGLDTVSGILSA |
| (uniprot | ISASFILSNADADTRTKAAAGVELTTKVLGNVGKGISQYIIAQRAAQGLST |
| P08715) | SAAAAGLIASAVTLAISPLSFLSIADKFKRANKIEEYSQRFKKLGYDGDSL |
| GenBank: | LAAFHKETGAIDASLTTISTVLASVSSGISAAATTSLVGAPVSALVGAVTG |
| AAA98233.1 | IISGILEASKQAMFEHVASKMADVIAEWEKKHGKNYFENGYDARHAAFLE |
| | DNFKILSQYNKEYSVERSVLITQQHWDTLIGELAGVTRNGDKTLSGKSYI |
| | DYYEEGKRLEKKXDEFQKQVFDPLKGNIDLSDSKSSTLLKFVTPLLTPGE |
| | EIRERRQSGKYEYITELLVKGVDKWTVKGVQDKGAVYDYSNLIQHASVG |
| | NNQYREIRIESHLGDGDDKVFLSAGSANIYAGKGHDVVYYDKTDTGYLTI |
| | DGTKATEAGNYTVTRVLGGDVKVLQEVVKEQEVSVGKRTEKTQYRSYEF |
| | THINGKNLTETDNLYSVEELIGTTRADKFFGSKFTDIFHGADGDDLIEG |
| | NDGNDRLYGDKGNDTLSGGNGDDQLYGGDGNDKLIGVAGNNYLNGGDG |
| | DDEFQVQGNSLAKNVLFGGKGNDKLYGSEGADLLDGGEGDDLLKGGYGND |
| | IYRYLSGYGHHIIDDDGGKEDKLSLADIDFRDVAFKREGNDLIMYKGEGN |
| | VLSIGHKNGITFRNWFEKESGDISNHEIEQIFDKSGRIITPDSLKKALEY |
| | QQRNNKASYVYGNDALAYGSQGDLNPLINEISKIISAAGSFDVKEERTAA |
| | SLLQLSGNASDFSYGRNSITLTTSA (SEQ ID NO: 10) |
| Pro- | 1 gataatggtt catgctaccg ggcgaatgaa acacgtcagt tcgccaggat gttgggactt |
| HlyA | 61 gaaccgaaga acacggcagt gcggagtccg gagagtaacg gaataacaga gagcttcgtg |
| 2 from | 121 aaaacgataa agcgtgatta cataagtatc atgcccaaac cagacgggtt aacggcagca |
| e coli | 181 aagaaccttg cagaggcgtt cgagcattat aacgaatggc atccgcatag tgcgctgggt |
| gene | 241 tatcgctcgc cacgggaata tctgcggcac ggggccagta atgggttaag tgataacagg |
| genbank | 301 tatctggaaa tataggggca aatccacctg gtcattatct ggaatttgac gaagtgtgat |
| M14107.1 | 361 aactggtata gccagattaa tctaaacctt tgtctgacaa aatcagataa agaagagtag |
| | 421 ttcaaaagac aactcgtgga ctctcattca gagagatagg cgttaccaaa atttgtttgg |
| | 481 aactgaacaa gaaaattgta tttgtgtaac tataatctta atgtaaaata aaagacacca |
| | 541 gttctgtaga atatgcttat tgaagagagt gtaataataa ttttatatag atgttgtaca |
| | 601 agacaggaat gagtaattat ttatgcttga tgttttttga ctcttgcttt ttatagttat |
| | 661 tatttttaag ttagtcagcg caataaaaac ttgcttttaa tattaatgcg agttatgaca |
| | 721 ttaaacggaa gaaacataaa ggcatatttt tgccacaata tttaatcata taatttaagt |
| | 781 tgtagtgagt ttattatgaa tataaacaaa ccattagaga ttcttgggca tgtatcctgg |
| | 841 ctatgggcca gttctccact acacagaaac tggccagtat ctttgtttgc aataaatgta |
| | 901 ttacccgcaa tacaggctaa ccaatatgtt ttattaaccc gggatgatta ccctgtcgcg |
| | 961 tattgtagtt gggctaattt aagtttagaa aatgaaatta aatatcttaa tgatgttacc |
| | 1021 tcattagttg cagaagactg gacttcaggt gatcgtaaat ggttcattga ctggattgct |
| | 1081 cctttcgggg ataacggtgc cctgtacaaa tatatgcgaa aaaaattccc tgatgaacta |
| | 1141 ttcagagcca tcagggtgga tcccaaaact catgttggta aagtatcaga atttcatgga |
| | 1201 ggtaaaattg ataaacagtt agcgaataaa atttttaaac aatatcacca cgagttaata |
| | 1261 actgaagtaa aaagaaagtc agattttaat ttttcattaa ctggttaaga ggtaattaaa |
| | 1321 tgacaacaat aaccactgca caaattaaaa gcacactgca gtctgcaaag caatccgctg |
| | 1381 caaataaatt gcactcagca ggacaaagca cgaaagatgc attaaaaaaa gcagcagagc |
| | 1441 aaacccgcaa tgcgggaaac agactcattt tacttatccc taaagattat aaaggacagg |
| | 1501 gttcaagcct taatgacctt gtcaggacgg cagatgaact gggaattgaa gtccagtatg |
| | 1561 atgaaaagaa tggcacggcg attactaaac aggtgttcgg cacagcagag aaactcattg |
| | 1621 gcctcaccga acggggagtg actatctttg caccacaatt agacaaatta ctgcaaaagt |
| | 1681 atcaaaaagc aggtaatata ttaggcggcg gtgctgaaaa tatcggtgat aacttaggaa |
| | 1741 aggcaggcgg tatattgtca acgtttcaaa atttcctggg tactgcactt tcctcaatga |
| | 1801 aaatagacga actgataaag aaacaaaaat ctggtggcaa tgtcagttct tctgaactgg |
| | 1861 caaaagcgag tattgagcta atcaatcaac ttgtggatac agttgccagc cttaataata |
| | 1921 atgttaactc attttctcaa caactcaata ctctgggaag tgtattatcc aatacaaagc |
| | 1981 acctgaacgg tgttggtaat aagttacaga atttacctaa tcttgataat atcggtgcag |
| | 2041 ggttagatac tgtatcgggt attttatctg cgatttcagc aagcttcatt ctgagcaatg |
| | 2101 cagatgcaga taccagaact aaagctgcag caggtgttga attaacaacg aaagtactgg |
| | 2161 gtaatgttgg aaaaggtatt tctcaatata ttatcgcaca gcgcgctgca caggggttat |
| | 2221 ctacatctgc tgctgctgcc ggtttaattg cttctgctgt gacactagca attagtcccc |
| | 2281 tctcattcct gtccattgcc gataagttta aacgtgccaa taaaatagag gagtattcac |
| | 2341 aacgattcaa aaaacttgga tacgatggtg acagtttact tgctgctttc cacaaagaaa |
| | 2401 caggggctat tgatgcatcg ttaacaacga taagcactgt actggcttca gtatcttcag |
| | 2461 gtattagtgc tgctgcaacg acatctctgg ttggtgcacc ggtaagcgcg ctggtaggcg |
| | 2521 ctgttacggg gataatttca ggtatccttg aggcttcaaa gcaggcaatg tttgaacatg |
| | 2581 ttgccagtaa aatggctgat gttattgctg aatgggagaa aaaacacggc aaaaattact |
| | 2641 ttgaaaatgg atatgatgcc cgccatgctg catttttaga agataacttt aaaatattat |
| | 2701 ctcagtataa taaagagtat tctgttgaaa gatcagtcct cattacccag caacattggg |
| | 2761 atacgctgat aggtgagtta gctggtgtca ccagaaatgg agacaaaaca ctcagtggta |
| | 2821 aaagttatat tgactattat gaagaaggaa aacgtctgga gaaaaaaycg gatgaattcc |
| | 2881 agaagcaagt ctttgaccca ttgaaaggaa atattgacct ttctgacagc aaatcttcta |
| | 2941 cgttattgaa atttgttacg ccattgttaa ctcccggtga ggaaattcgt gaaaggaggc |
| | 3001 agtccggaaa atatgaatat attaccgagt tattagtcaa gggtgttgat aaatggacgg |
| | 3061 tgaagggggt tcaggacaag ggggctgtat atgattactc taacctgatt cagcatgcat |
| | 3121 cagtcggtaa taaccagtat cgggaaattc gtattgagtc acacctggga gacggggatg |
| | 3181 ataaggtctt tttatctgcc ggctcagcca atatctacgc aggtaaagga catgatgttg |
| | 3241 tttattatga taaaacagac accggttatc tgaccattga tggcacaaaa gcaaccgaag |
| | 3301 cgggtaatta cacggtaaca cgtgtacttg gtggcgatgt taaggtttta caggaagttg |
| | 3361 tgaaggagca ggaggtttca gtcggaaaaa gaactgaaaa aacgcaatat cggagttatg |
| | 3421 aattcactca tatcaatggt aaaaatttaa cagagactga taacttatat tccgtggaag |
| | 3481 aacttattgg gaccacgcgt gccgacaagt tttttggcag taaatttact gatatcttcc |
| | 3541 atggcgcgga tggtgatgac cttatagaag gaaatgatgg gaatgaccgc ttatatggtg |
| | 3601 ataaaggtaa tgacacactg agtggtggaa acggagatga ccagctctat ggcggtgatg |
| | 3661 gtaacgataa gttgattggg gtagcaggta ataattacct gaacggcgga gatggagatg |
| | 3721 atgagtttca ggttcaggga aattctcttg caaaaaatgt attattcggt ggaaaaggta |
| | 3781 atgacaagct gtacggcagt gagggggcag atctgcttga tggtggagag ggggatgatc |
| | 3841 tcctgaaagg cggatatggt aatgatattt atcgttatct gtcaggatat ggtcatcata |
| | 3901 ttattgatga tgatgggggg aaagaggata aactcagttt ggctgatatt gatttccggg |
| | 3961 atgtggcctt caagcgagaa ggtaatgacc tcatcatgta taaaggtgaa ggtaatgttc |
| | 4021 tttccattgg tcataaaaat ggtattacat tcaggaactg gtttgaaaaa gagtcaggtg |
| | 4081 atatctctaa tcacgagata gagcagattt ttgataaaag tggccggata atcacacctg |
| | 4141 attcccttaa aaaggcactt gagtatcaac agcgtaataa taaggcaagt tatgtgtatg |
| | 4201 ggaatgatgc attagcctat ggaagtcagg gtgatcttaa tccattaatt aatgaaatca |
| | 4261 gcaaaatcat ttcagctgca ggtagcttcg atgttaaaga ggaaagaact gcagcttctt |
| | 4321 tattgcagtt gtccggtaat gccagtgatt tttcatatgg acggaactca ataaccctga |
| | 4381 ccacatcagc ataatatatt aatttaaatg atagcaatct tactgggctg tgccacataa |
| | 4441 gattgctatt ttttttggag tcataatgga ttcttgtcat aaaattgatt atgggttata |
| | 4501 cgccctggag attttagccc aataccataa cgtctctgtt aacccggaag aaattaaaca |
| | 4561 tagatttgat acagacggga caggtctggg attaacgtca tggttgcttg ctgcgaaatc |
| | 4621 tttagaacta aaggtaaaac aggtaaaaaa aacaattgat cgattaaact ttatttttct |
| | 4681 gcccgcatta gtctggagag aggatggacg tcattttatt ctgactaaaa tcagcaaaga |
| | 4741 agtaaacaga tatcttattt ttgatttgga gcagcgaaat ccccgtgttc tcgaacagtc |
| | 4801 tgagtttgag gcgttatatc aggggcatat tattcttatt acttcccgtt cttctgttac |
| | 4861 cgggaaactg gcaaaatttg actttacctg gtttattcct gccattataa aatacaggag |
| | 4921 aatatttatt gaaacccttg ttgtatctgt ttttttacaa ttatttgcat taataacccc |
| | 4981 cctttttttt caggtggtta tggacaaagt attagtgcac agggggtttt caacccttaa |
| | 5041 tgttattact gttgccttat ctgttgtagt ggtgtttgag attatactca gcggtttaag |
| | 5101 aacttacatt tttgcacata gtacaagtcg gattgatgtt gagttgggtg ccaaactctt |
| | 5161 ccggcattta ctggcgctac cgatctctta ttttgagagt cgtcgtgttg gtgatactgt |
| | 5221 tgcgagggta agagaattag accagatccg taattttctg acaggacagg cattaacatc |
| | 5281 tgttttggac ttattatttt cactcatatt ttttgcggta atgtggtatt acagcccaaa |
| | 5341 gcttactctg gtgatcttat tttcgctgcc ttgttatgct gcatggtctg tttttattag |
| | 5401 ccccattttg cgacgtcgcc ttgatgataa gttttcacgg aatgcggata atcaatcttt |
| | 5461 cctggtggaa tcagtaacgg cgattaacac tataaaagct atggcagtct cacctcagat |
| | 5521 gacgaacata tgggacaaac aattggcagg atatgttgct gcaggcttta aagtgacagt |
| | 5581 attagcaacc attggtcaac aaggaataca gttaatacaa aagactgtta tgatcatcaa |
| | 5641 cctatggttg ggagcacacc tggttatttc cggggattta agtattggtc agttaattgc |
| | 5701 ttttaatatg cttgctggtc agattgttgc accggttatt cgccttgcac aaatctggca |
| | 5761 ggatttccag caggttggta tatcagttac ccgccttggt gatgtgctta actctccaac |
| | 5821 tgaaagttat catgggaaac tgacattgcc ggaaattaat ggtgatatca cttttcgtaa |
| | 5881 tatccggttt cgctataaac ctgattctcc ggttattttg gacaatatca atcttagtat |
| | 5941 taagcagggg gaggttattg gtattgtcgg acgttctggt tcaggaaaaa gcacattaac |
| | 6001 taaattaatt caacgttttt atattcctga aaatggccag gtattaattg atggacatga |
| | 6061 tcttgcgttg gccgatccta actggttacg tcgtcaggtg ggggttgtgt tgcaggacaa |
| | 6121 tgtgctgctt aatcgcagta ttattgataa tatttcactg gctaatcctg gcatgtccgt |
| | 6181 cgaaaaagtt atttatgcag cgaaattagc aggtgctcat gattttattt ctgaattgcg |
| | 6241 tgaggggtat aacaccattg tcggggaaca gggggcagga ttatccggag gtcaacgtca |
| | 6301 acgcatcgca attgcaaggg cgctggtgaa caaccctaaa atactcattt ttgatgaagc |
| | 6361 aaccagtgct ctggattatg agtcggagca tgtcatcatg cgcaatatgc acaaaatatg |
| | 6421 taagggcaga acggttataa tcattgctca tcgtctgtct acagtaaaaa atgcagaccg |
| | 6481 cattattgtc atggaaaaag ggaaaattgt tgaacagggt aaacataagg agctgctttc |
| | 6541 tgaaccggaa agtttataca gttacttata tcagttacag tcagactaac agaaagaaca |
| | 6601 gaagaatatg aaaacatggt taatggggtt cagcgagttc ctgttgcgct ataaacttgt |
| | 6661 ctggagtgaa acatggaaaa tccggaagca attagatact ccggtacgtg aaaaggacga |
| | 6721 aaatgaattc ttacccgctc atctggaatt aattgaaacg ccagtatcca gacggccgcg |
| | 6781 tctggttgct tattttatta tggggtttct ggttattgct tttattttat ctgttttagg |
| | 6841 ccaagtggaa attgttgcca ctgcaaatgg gaaattaaca cacagtgggc gtagtaaaga |
| | 6901 aattaaacct attgaaaact caatagttaa agaaattatc gtaaaagaag gagagtcagt |
| | 6961 ccggaaaggg gatgtgttat taaagcttac agcactggga gctgaagctg atacgttaaa |
| | 7021 aacacagtca tcactgttac aggccaggct ggaacaaact cggtatcaaa ttctgagcag |
| | 7081 gtcaattgaa ttaaataaac tacctgaact aaagcttcct gatgagcctt attttcagaa |
| | 7141 tgtatctgaa gaggaagtac tgcgtttaac ttctttgata aaagaacagt tttccacatg |
| | 7201 gcaaaatcag aagtatcaaa aagaactgaa tttggataag aaaagagcag agcgattaac |
| | 7261 agtacttgcc cgtataaacc gttatgaaaa tttatcaagg gttgaaaaaa gccgtctgga |
| | 7321 tgatttcagt agtttattgc ataaacaggc aattgcaaaa catgctgtac ttgagcagga |
| | 7381 gaataaatat gtcgaagcag taaatgaatt acgagtttat aaatcacaac tggagcaaat |
| | 7441 tgagagtgag atattgtctg caaaagaaga atatcagctt gttacgcagc tttttaaaaa |
| | 7501 tgaaatttta gataagctaa gacaaaccac agacaacatt gggttattaa ctctggaatt |
| | 7561 agcgaaaaat gaagagcgtc aacaggcttc agtaatcagg gccccagttt cggtaaaagt |
| | 7621 tcagcaactg aaggttcata ctgaaggtgg ggttgttaca acagcggaaa cactgatggt |
| | 7681 catcgttccg gaagatgaca cgctggaggt tactgctctg gtacaaaata aagatattgg |
| | 7741 ttttattaac gtcgggcaga atgccatcat taaagtggag gcatttcctt atacacgata |
| | 7801 tggttatctg gtgggtaagg tgaaaaatat aaatttagat gcaatagaag accagagact |
| | 7861 gggacttgtt tttaatgtta ttatttctat tgaagagaat tgtttgtcaa ccggaaataa |
| | 7921 aaacattcca ttaagctcgg gtatggcagt cactgcagaa ataaagacag gtatgcgaag |
| | 7981 tgtaatcagt tatcttctta gtcctttaga agagtcagta acagaaagtt tacgtgagcg |
| | 8041 ttaagtttca gaagtccagt atttgctgct atacgtgctg cgtggcactt gccgtctgaa |
| | 8101 cggcttgatc cggaagccaa gtcaaacaac agcgtgatga gcgtcagggc aaacaccaag |
| | 8161 gctctctcga tgacaccaga acaaattgaa tacgtgctag gaaaagctac cgagt |
| | (SEQ ID NO: 11) |

The sequences of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 (or any derivable range therein) or more mutations (substitutions or deletions) or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous sequences, or any range derivable therein, of the SEQ ID NOs: 1-11, respectively.

### EXAMPLES

The following examples are included to demonstrate at least some embodiments of the disclosure. It should be appreciated by those of skill in the art that the techniques disclosed in the examples that follow represent techniques discovered to function well in the practice of the subject matter of the disclosure, and thus can be considered to constitute particular modes for its practice. However, those of skill in the art should, in light of the present disclosure, appreciate that many changes can be made in the specific embodiments that are disclosed and still obtain a like or similar result without departing from the spirit and scope of the inventions encompassed herein.

### Example 1

### Materials and Methods

### Bacterial Strains and Culture Conditions

The E. coli strains utilized in this study were obtained from a single colony grown on the Lysogeny Broth (LB) plate (10 g/L tryptone, 0.5 g/L sodium chloride (NaCl), and 5 g/L yeast extract). Bacterial cultures were incubated at 37° C. after resuscitation from a frozen stock (−80° C., 10% glycerol). The ExPEC ST131 strains used in the study, JJ1886, JJ1901, JJ2050, JJ2528, and JJ2547, were kindly provided by James R. Johnson [78]. Uropathogenic E. coli (UPEC) strains UTI89 (O18:K1:H7, ST95) and CFT073 (O6:K2:H1; ST73) were kindly provided by Kathryn Patras. E. coli strains W0008 (ST127-like), W0044 (ST405-like), W0128 (ST648-like) were isolated from the blood or feces of hospitalized patients with bacteremia. The number of colony-forming units (CFU) administered was determined by correlating the optical density (OD) at 600 nm to the number of colonies observed after plating.

The hlyA Sequence Distribution and HlyA Alignment

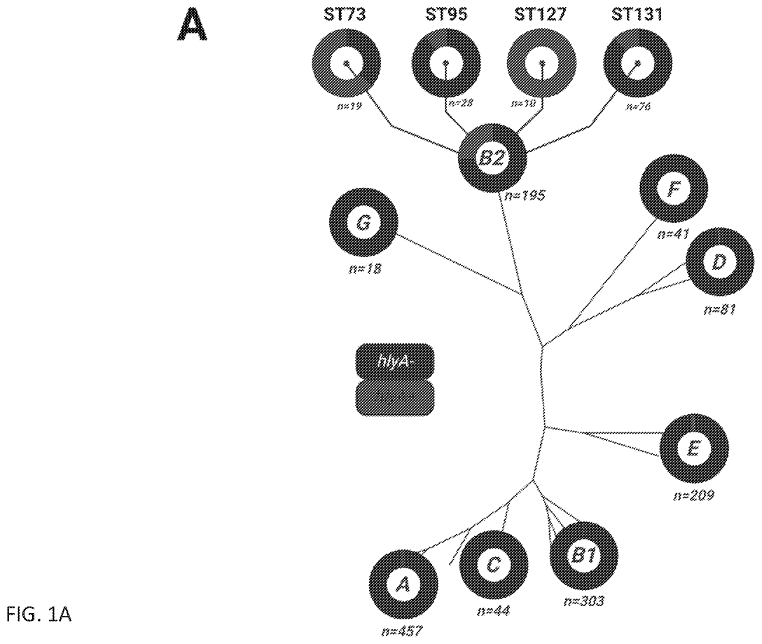

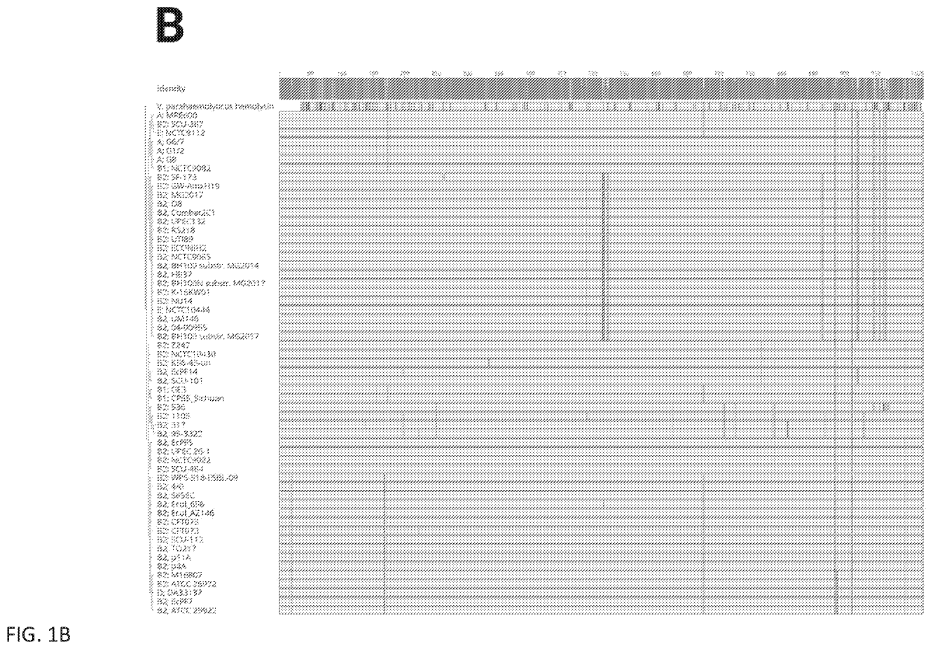

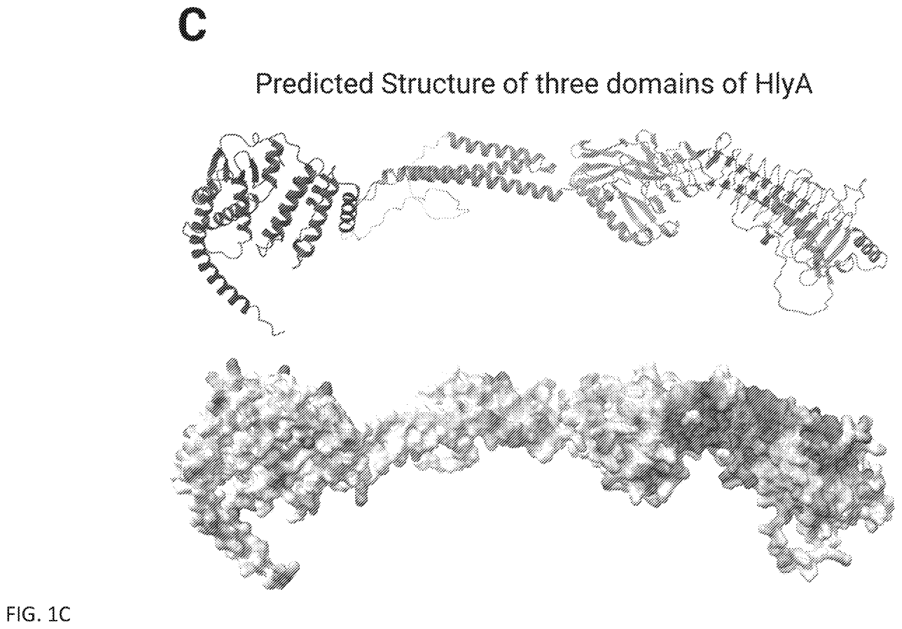

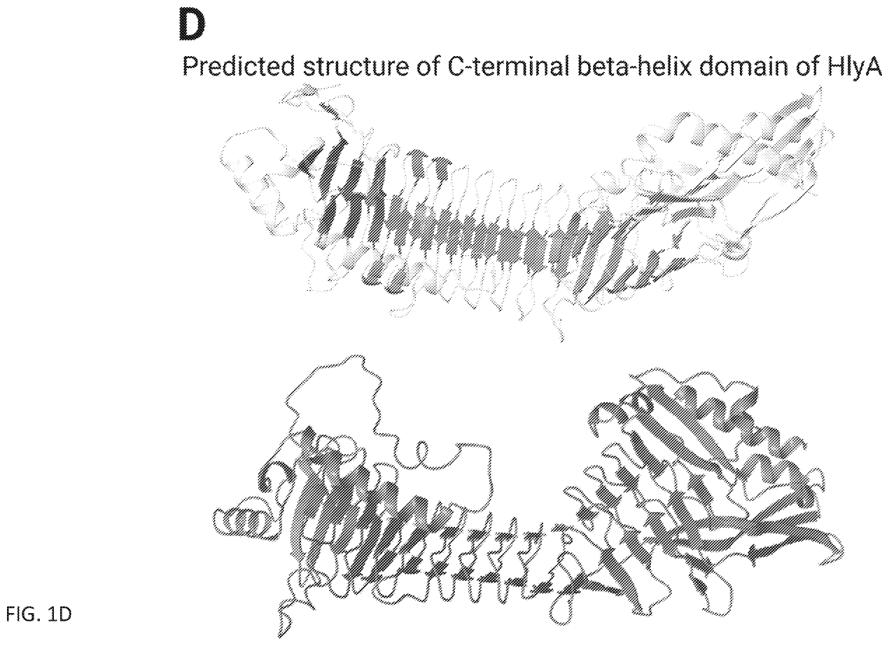

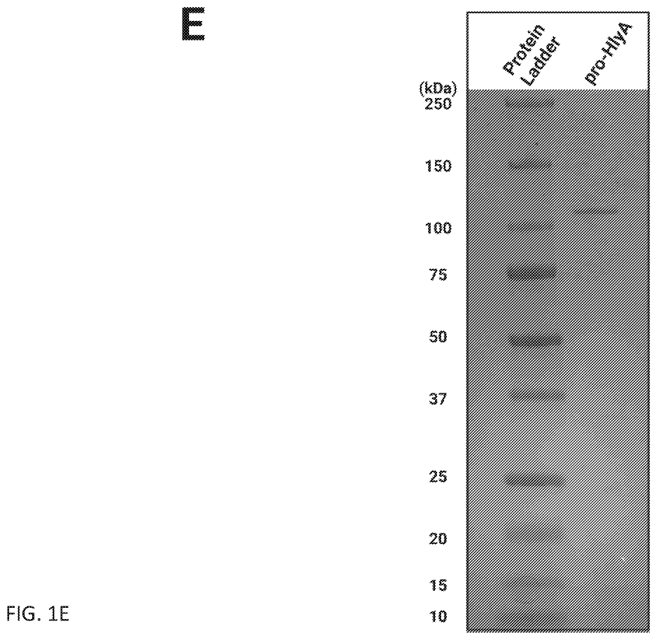

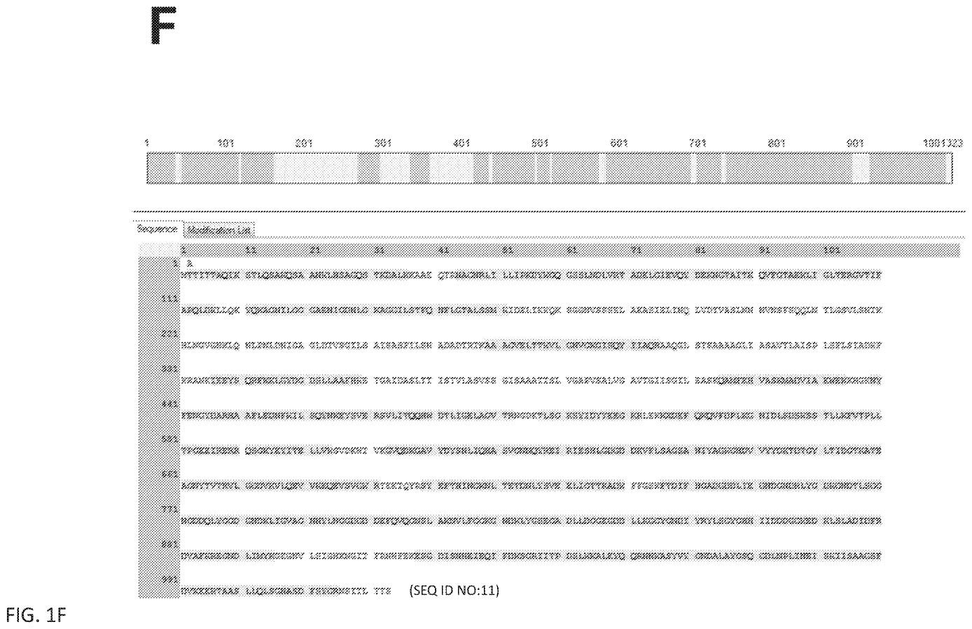

Complete E. coli genomes were obtained from NCBI's RefSeq database and sorted into phylogroups as previously reported [75]. The sorted genomes were then categorized by sequence types using the MLST software (https://github.com/tseemann/mlst”) which uses the PubMLST databases (https://pubmlst.org/) [82]. The categorized genome database was then used as a custom BLAST (version 2.8.1) [83-85] database to search for hits to the hlyA, hlyB, hlyC, and hlyD genes from the hly operon of E. coli UTI89 genome (accession: CP000243.1). To design FIG. 1A, the underlying phylogenetic tree diagram was created using the autoMLST software in concatenated alignment mode with 1,000 UltraFast Bootstrap replicates using representative genomes from the 8 E. coli phylogroups. The representative phylogenetic diagram was then overlaid with pie charts created in GraphPad Prism version 9.5.0 using BLAST hit results. Finally, the figures were combined using Biorender. All software used default settings unless otherwise specified. Open reading frames that overlapped with BLAST hits (described above) were extracted and translated using the bacterial translation code (translation table 11) in Geneious 2023.1.1. Translated HlyA sequences were then aligned using Geneious Alignment Software with free end gaps and otherwise default settings after truncated ORFs were removed. The resulting alignment was sorted by a phylogenetic tree annotation that was created using RXaML (version 8.1.1) with the GAMMA BLOSUM62 protein model and 100 Bootstrap replicates from the Rapid Bootstrap algorithm to create a consensus tree. Vibrio parahaemolyticus hemolysin A (accession: WP_041955411.1) was used as an outgroup.

Prediction of Protein Structure for HlyA with AlphaFold2

The nucleotide sequence of HlyA was used to recreate the translated amino acid sequence using ExPASy. All six possible reading frames (three forward, three backward) were generated, and the frame that had the sequence for complete HlyA was used as the amino acid sequence for structure prediction. ColabFold's AlphaFold2-Advanced Google Notebook (Google, Mountain View, CA) was used to generate predictions from amino acid sequences [87,88]. For multiple sequence alignment (MSA) necessary to build the consensus model for the structure of HlyA, we used MMseq2 [89,90]. Five prediction runs were run, with each run using a randomly chosen initiation point for the start of prediction runs. These models were ranked using the following two metrics: 1. pLDDT (predicted IDDT-Ca) with its ability to quantify the confidence of model per residue calculated by utilizing distances between Ca atoms in multiple reference models, and 2. AlphaFold-generated PAE (Predicted Aligned Error) for every residue, a numerical value of expected position error per residue [87,91]. The model with the highest average pLDDT and lowest PAE was chosen as the best-predicted structure of HlyA, and Amber Force Field was applied to relax the structure [92]. The predicted structure was compared against the list of previously solved structures of RTX toxins deposited on PDB aligning spatial coordinates of models by domains [93,94]. Additionally, Foldseek search was used to search for similar solved and AlphaFold-predicted structures on the AFDB-Swiss-Prot database through UniProt [95,96]. Additionally, these structures aligned with the predicted structure by UCSF ChimeraX's alignment feature using the Needleman-Wunsch algorithm with BLOSUM-62 similarity matrix [97]. ChimeraX was used for analyzing the structural features of the predicted model, determining local physical properties within domains, and visualizing the model.

### Plasmid Construction

The plasmid for the candidate vaccine antigen SinH-3 was constructed using a previously described method [77]. The plasmids pSU-hlyA (encoded the candidate vaccine antigen pro-HlyA, Uniprot entry: P08715), and pK184-hlyBD (encoded the necessary transport complex components HlyB and HlyD) were kindly provided by Lutz Schmitt [76]. The mRNA plasmid was constructed by cloning the SinH-3 gene from ExPEC sequence type 131 (ST131) strain JJ1887 genomic DNA (SinH-Ig-like domains-3, encoding the C-terminal passenger Ig-like domain-3 fragment of sinH, amino acid residues 602 to 724) and the pro-HlyA was cloned from E. coli (Uniprot entry: P08715). Both sequences (hlyA and sinH-3) were submitted to Creative Biolabs for constructing a sinH-3: hlyA mRNA construct with IL-2 signal peptide and P2A self-cleaved sequence, called pIVTScrip-mRNA-IL2-sig_hlyA-P2A-IL2-sig_sinH (hereinafter named Dual-Hit mRNA construct). The Dual-Hit mRNA constructs were further enclosed in Cationic Lipid Nanoparticle (Cationic Lipid Nanoparticle (SM-102/DSPC/Cholesterol/DMG-PEG=50:10:38.5:1.5) and stored in Tris-based buffer at −80° C.

### Vaccine Antigens Preparation

The recombinant SinH-3 protein was expressed as fusions with glutathione-S-transferase (GST) using E. coli BL21 (DE3) and purified as previously described [77]. To purify the recombinant protein pro-HlyA, the plasmid pSU-hlyA, containing the C-terminal secretion signal of HlyA (Uniprot entry: P08715), and pK184-hlyBD, encoding HlyB and HlyD essential for the transport complex, were co-transformed into E. coli BL21 (DE3) cells [76]. A single pSU-hlyA and pK184-hlyBD co-transformed E. coli BL21 (DE3) colony was used to inoculate a 300 ml baffled flask containing 150 ml of Lysogeny broth (LB) medium and cultured overnight. The overnight culture was then used to inoculate a 2 L baffled flask containing 800 ml of LB medium, which was grown at 37° C. until it reached optical density at 600 nm (OD600) of 0.4-0.6. Gene expression was induced with 1 mM Isopropyl β-D-1-thiogalactopyranoside (IPTG) (Sigma-Aldrich, St. Louis, MO), and the culture was incubated overnight at 37° C. and 150 rpm. Following induction, the supernatant containing secreted pro-HlyA protein was collected by centrifugation (Thermo Scientific, Sorvall RC 6+, SLA-3000 (Rotor), 10,000×g for 30 min at 4° C.) and filtered through the 0.22 μm Vacuum Driven Sterile Filters (Sigma-Aldrich, St. Louis, MO). The filtered supernatant was subsequently concentrated to 1 ml using Amicon Ultra-15 Centrifugal Filter Units (Millipore Sigma, Burlington, MA) with a 100 kDa molecular-weight cut-off (MWCO). All antigens were analyzed by SDS-PAGE and Coomassie Brilliant Blue staining, and expression of both purified proteins was confirmed by mass spectrometry as previously described [77]. Both recombinant proteins were stored at −20° C. until further use and handled at 4° C. The control group consisted of culturing a single untransformed E. coli BL21 (DE3) colony only (hereinafter referred to as control supernatant). The resulting supernatant was collected and concentrated using the exact same procedure as that used for the purification of pro-HlyA above.

### Experimental Animals

Experimental animals used in this study were 6-8 weeks old BALB/cJ mice obtained from Jackson Laboratories (Bar Harbor, ME). They were provided with sterile food and water ad libitum and housed in filtered cages with 3-4 mice per cage. All experimental procedures performed on mice were approved in accordance with relevant guidelines and regulations from “The Guide and Care and Use of Laboratory Animals” (National Institute of Health) and were approved by Baylor College of Medicine's Institutional Animal Care and Use Committee under protocol number AN-5177.

### Vaccination

For experiments involving protein-subunit vaccines, purified proteins were mixed with alum adjuvant (G-Bioscience, St. Louis, MO) in a 2:1 ratio of antigen to adjuvant, following the manufacturer's guidelines. Female BALB/cJ mice (6-8 weeks old) were given three subcutaneous injections (S.C) of either 50 μg of pro-HlyA or a mixture of SinH-3 and pro-HlyA (50 μg each) on days 0, 14, and 28. Control groups were either given vaccinations control samples (comprising the same volume of control supernatant (30 μl), described above, mixed with alum adjuvant (30 μl) or left unvaccinated. In experiments involving mRNA vaccines, 6-week-old male BALB/cJ mice were given three intramuscular injections (I.M) of either 2 μg Dual-Hit mRNA construct (low-dose group, 40 μl to one hind leg muscle), 5 μg Dual-Hit mRNA construct (high-dose group, 40 μl to one hind leg muscle) [98], or 50 μl of Tris-based buffer (control group).

### Murine Model of Bacteremia (UTI89)

E. coli strains UTI89 were cultured under specified conditions one day prior to injection. On the day of injection (day 42), the strains were subcultured in LB broth at a ratio of 1:100 to an OD600 of approximately 0.6 (Log phase, ~1×10⁸ CFU/ml), harvested by centrifugation (3,500×g for 20 min at 4° C., Centrifuge 5702 R, Eppendorf North America, Framingham, MA), and suspended in an equivalent amount of 1×PBS. Mice were intraperitoneally injected with 50 μl of the E. coli strain suspension (1×10⁸ CFU) on day 42, and the inoculum was quantified by plating dilutions onto LB agar. After 16-hours, mice were euthanized and necropsied to collect their kidney, spleen, and liver. The organs were homogenized in 1 ml 1×PBS using a BeadBlaster Refrigerated Homogenizer (Benchmark Scientific Inc, Sayreville, NJ, USA), and the organ homogenates were plated on LB agar plates and incubated at 37° C. to determine the number of bacteria or colony-forming units (CFU) per milliliter (mL).

### Murine Model of Mortality (UTI89 or CFT073)

On the day before injection, E. coli strains UTI89 and CFT073 were grown under the specified conditions. On the day of injection (day 42), mice were intraperitoneally injected with 50 μl of either UTI89 (5×10⁷ CFU) or CFT073 (1×10⁸ CFU) suspension. Mice were closely monitored twice daily for ten days to observe morbidity and mortality. Survival data were collected over time, and moribund or dead animals were euthanized and necropsied to determine bacterial levels in their kidney, spleen, and liver. The organs were homogenized in 1 ml 1×PBS using a BeadBlaster Refrigerated Homogenizer, and the organ homogenates were plated on LB agar plates and incubated at 37° C. to determine the number of bacteria or CFU per milliliter (mL). Moribundity was determined based on multiple observable features, including rough coat, hunched posture, lethargy, and hyperpnea.

### Murine Model of Urinary Tract Infection (UTI89 or CFT073)

UPEC strains UTI89 and CFT073 were grown and prepared as previously described. On day 42, mice were transurethrally inoculated with 50 μl of a UPEC strain suspension (1×10⁸ CFU). The inoculum was quantified by plating dilutions onto LB agar. After 72 hours, mice were euthanized and necropsied to collect bladders. The bladders were homogenized in 500 μl 1×PBS using a BeadBlaster Refrigerated Homogenizer, and the organ homogenates were plated on LB agar plates and incubated at 37° C. to determine the number of bacteria or CFU per milliliter (mL).

Murine Model of Mortality from Mixture of Ten Strains

Ten E. coli strains, representing typical sequence types (STs) of ExPEC, were grown and prepared as previously described. On day 42, mice were intraperitoneally injected with 50 μl of a mixture of ten ExPEC strains (equally mixed, a total of 1×10⁸ CFU). The inoculum was quantified by plating dilutions on LB agar. Mice were monitored twice daily for ten days to observe their survival. Survival data were collected over time, and moribund or dead mice were euthanized and necropsied to determine bacterial levels in their kidney, spleen, and liver. The organs were homogenized in 1 ml 1×PBS using a BeadBlaster Refrigerated Homogenizer, and the homogenates were plated on LB agar plates and incubated at 37° C. to determine the number of bacteria with CFU per milliliter (mL). Moribundity was determined by observing multiple features, including rough coat, hunched posture, lethargy, and hyperpnea.

### Statistical Analyses

Graphs and statistical analyses were conducted using GraphPad Prism version 9 (GraphPad Software, San Diego, CA). Significance was determined using the Mann-Whitney U test (two groups) or Kruskal-Wallis analysis of variance (ANOVA) with Dunn's multiple comparisons correction (more than two groups). Survival curves were compared using the Genhan-Breslow-Wilcoxon curve comparison. A 95% confidence interval was used for all statistical analyses, with alpha values of 0.05. Statistical significance was determined if the calculated P-values were less than 0.05. The lines of all the bar graphs were at the median with 95% confidence intervals (CI). If no bacterial colony is detected on the plate, the CFU count will be calibrated from 0 to 1, thereby indicating the value in logarithmic form in the figures. The statistical significance is represented as one star (*) for P<0.05, two stars (**) for P<0.01, three stars (***) for P<0.001, and four stars (****) for P<0.0001. The box-and-whisker plots and Kaplan Meier survival curves were generated using GraphPad Prism 9 and annotated with BioRender.

### Example 2

### Comparative Genomics of Hemolysin (HlyA) in ExPEC

The vaccine search efforts center around a strategy to use comparative pathogenomics combined with functional vaccine antigen characterization to identify the best candidates for development. Some criteria include searching for genes that encode proteins that are surface or extracellularly secreted (for the immune system to access), are involved in the pathogenesis of the organism, are likely involved in disease-specific symptomology, are expressed during infection, and are prominent in disease-causing strains. Using a database of 1,348 complete E. coli genomes and >20,000 genomes available in public databases, we settled on a putative hemolysin toxin, HlyA, as a candidate worthy of exploration. Although only present in 65 genomes when blasted for the hlyA sequence (of the 1,348) [75], the phylogroup distribution of the hlyA sequence shows it is predominantly found in phylogroup B2, specifically the ExPEC and UPEC-associated sequence types (STs) ST73, ST95, ST127, and ST131 (FIG. 1A). Alignment and phylogenetic analysis of the amino acid sequences of HlyA suggests HlyA is highly conserved, with all alleles being >97% identical on the amino acid level (FIG. 1B). These results also suggest that the hlyA sequence has a horizontal pattern of transmission between phylogroups, as the alleles of hlyA within non-B2 phylogroups are nested within those of the B2 phylogroup (FIG. 1B).

### Example 3

### Structural Prediction of HlyA

The AlphaFold2-predicted protein structure of HlyA reveals structural and organizational parallels with previously characterized RTX toxins. HlyA is predicted to comprise three domains: a putative N-terminal adenylate cyclase (residues 1-279, red), a three-helix bundle (residues 321-437, blue), a predominantly beta-helix C-terminal domain (residues 438-1023, green), and a linker connects the adenylate cyclase and helix-bundle domains (residues 280-320, grey) (FIG. 1C). The electrostatic map of this region suggests that HlyA has the capability to form membrane pores as previously seen in RTX toxins at higher concentrations; this function would be vulnerable to disruption by steric interactions from antibodies that bind to this domain. A predominant feature of the C-terminal domain is two-strand beta helix repeats that span the length of this domain (FIG. 1D). This domain bears striking beta-helical structural similarity to highly immunogenic virulence factor pertactin from B. pertussis, which is universally used as one of the immunogenic components necessary for efficacy in acellular pertussis vaccines [99,100]. Furthermore, it has been shown that neutralizing antibodies can disrupt the interaction between RTX leukotoxin and host integrin receptors by targeting the beta helix-repeat domain mediating host-pathogen interaction (hemolysin A from P. mirabilis (top, PDB: 4W8Q) and RTX fragment from B. pertussis AC toxin (bottom, PDB: 7RAH) [101,102]. This potentially offers an additional way to interfere with ExPEC-host interaction that could work in synergy with vaccines with similar modes of action.

### Example 4

### HlyA Function, Expression and Purification

The activation and secretion of HlyA are regulated by the hlyCABD operon, comprising the acyltransferase HlyC, the ABC transporter HlyB, and the outer membrane fusion protein HlyD [103]. The secretion process can be described by the interaction of HlyA with the pre-assembled HlyBD complex, which prompts contact with TolC, a multifunctional outer membrane protein (OMP) of E. coli. This interaction forms a trans-periplasmic export channel, capable of directly transporting substrates (HlyA) from the cytoplasm to the extracellular medium, without the formation of periplasmic intermediate [104,105]. Recently, cryo-electron microscopy (cryo-EM) structures determined that the inner membrane complex formed by HlyB and HlyD is a hetero-dodecameric assembly composed of three HlyB homodimers and six HlyD subunits. Functional studies have further validated that oligomerization of HlyB and HlyD is critical for protein (HlyA) secretion [106]. HlyA is a member of the RTX toxin family and possesses the ability to form a pore in the membranes of various cell types [107]. However, its maturation from a non-toxic precursor, pro-HlyA, into an active toxin, necessitates a fatty acylation at two internal lysine residues (Lys 540 and Lys 648), facilitated by the acyltransferase HlyC [108]. This lipidation is not required for secretion, but rather for hemolytic and cytotoxic activity. In the absence of acylation, the inactive precursor of HlyA, pro-HlyA, fails to induce pore formation in the host cell membrane and does not induce calcium flux [59].

Given these reasons and with the goal of determining a safe potential vaccine candidate against ExPEC, it was decided to use the non-acylated, inactive form of HlyA, pro-HlyA, as the vaccine antigen candidate, and used the co-transformation expression system (involving HlyBD and HlyA, with the sequence of hlyC deleted) to purify the protein. The plasmids utilized were hlyA sequence (Uniprot entry: P08715) was cloned into plasmid pSU-hlyA, while transport complex components HlyB and HlyD were encoded into plasmid pK184-hlyBD [76]. Both plasmids were co-transformed into E. coli BL21 (DE3) cells. Bacterial cultures expressing recombinant pro-HlyA antigen secreted the protein into the supernatant, which was subsequently harvested, filtered, concentrated and visualized by sodium dodecyl sulfate-polyacrylamide gel electrophoresis (SDS-PAGE). Coomassie blue staining of the gels revealed a dominant band, presumed to be pro-HlyA (110 kDa) (FIG. 1E). The identity of the putative pro-HlyA protein was confirmed by mass spectrometry by MS/MS of the band. Purified protein bands were resolved and digested in gel, and the tryptic peptides were analyzed on a nanospray LC-MS (liquid chromatography-mass spectrometry) system. The coverage of the candidate antigen pro-HlyA, which is defined as the percentage of the protein sequence covered by identified peptides, was approximately 73%, with 97 peptides detected (FIG. 1F). This high sequence coverage substantiated the identity of the antigen, enabling its utilization in further experiments. The recombinant SinH-3 protein, fused to glutathione-S-transferase (GST), was expressed in E. coli BL21 (DE3) and purified following a previously described method [77].

### Example 5

### Pro-HlyA Vaccination in a Murine Model of Bacteremia

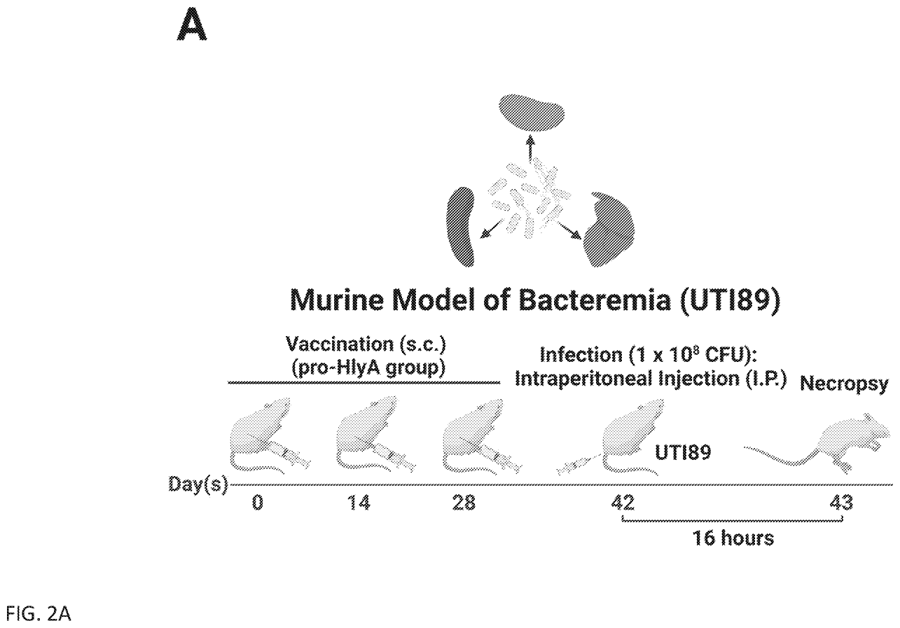

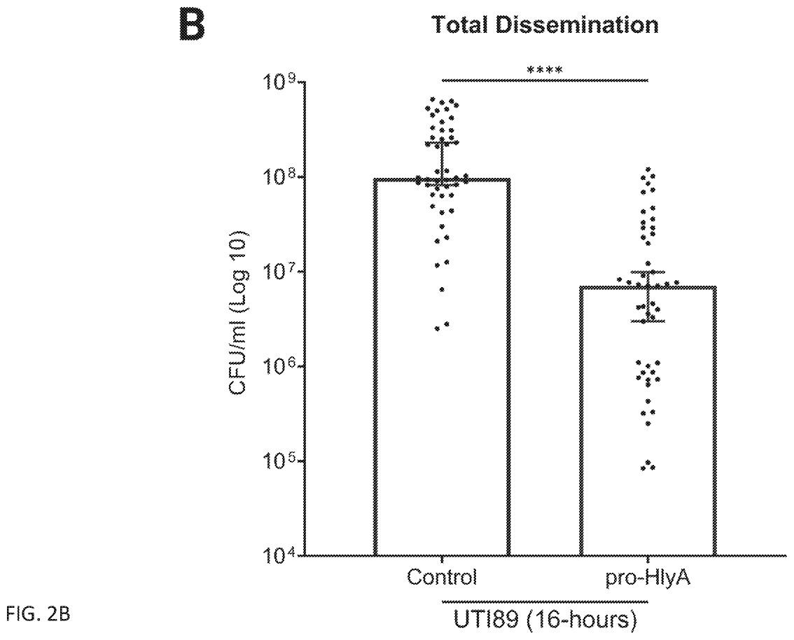

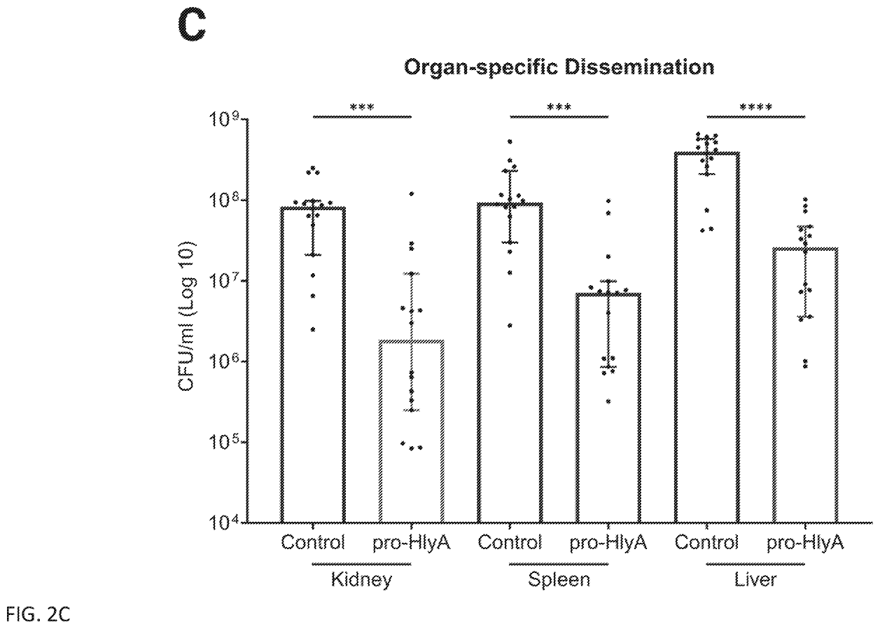

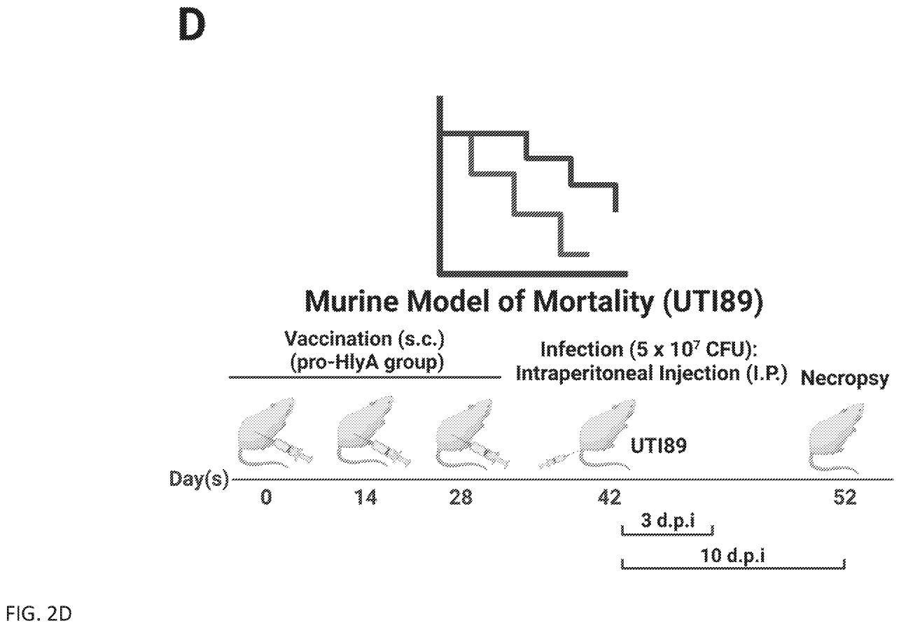

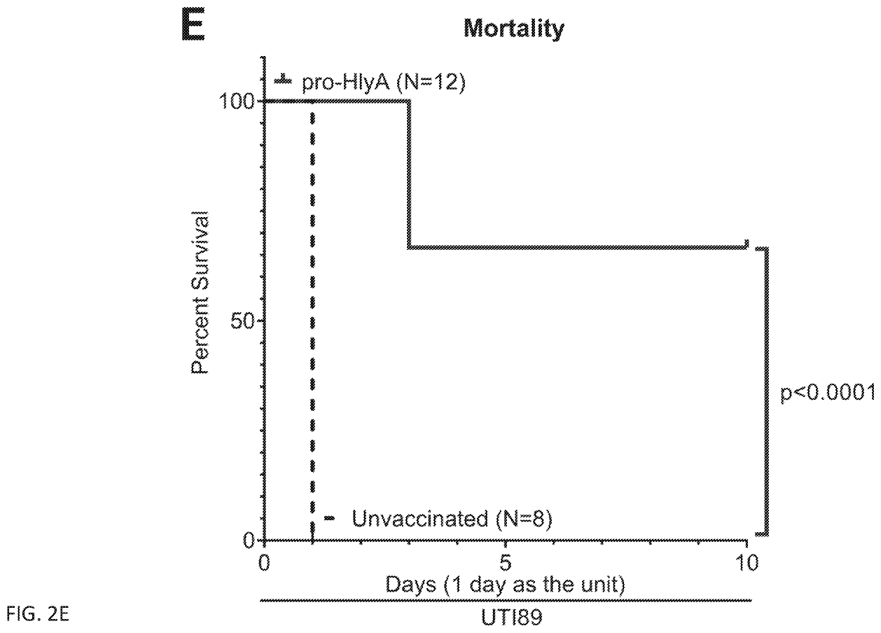

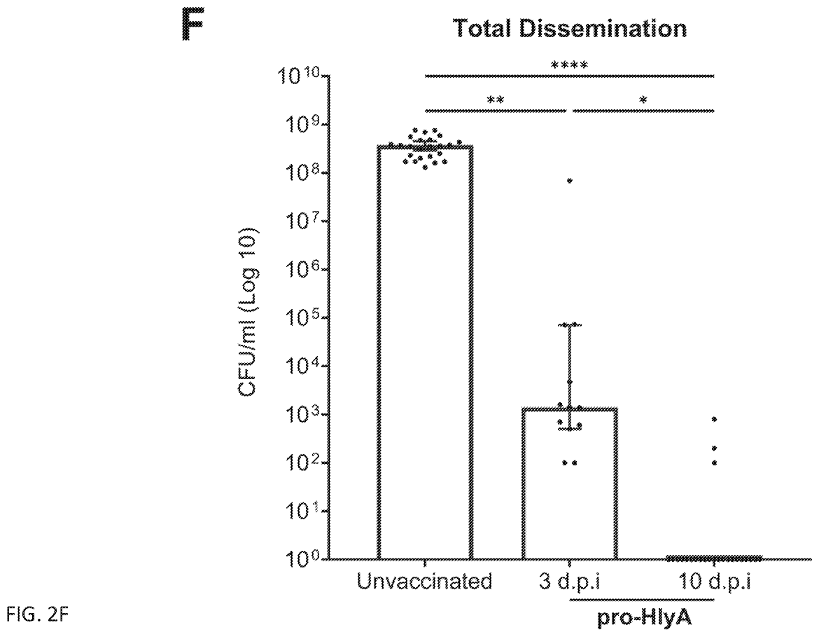

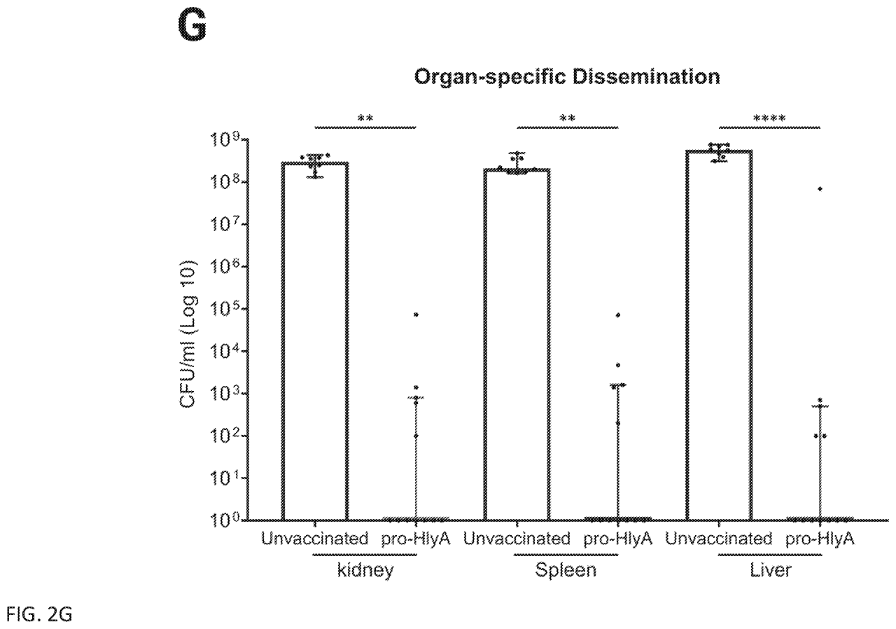

UTI89, an ExPEC strain belonging to multilocus sequence type 95 (ST95) [110], has been isolated from patients with urinary tract infections and acute cystitis [111]. ST95, along with ST73 and ST131, is predominantly found in ExPEC strains and represents the second most prevalent clonal group in patients with bloodstream infections (BSIs) [112]. To evaluate the protective efficacy of pro-HlyA in a UTI89 bacteremia model, mice were subcutaneously immunized with purified pro-HlyA combined with alum adjuvant (2:1 antigen/alum ratio), while the control group mice were injected with a mixture comprising equal volumes of control supernatant and alum adjuvant. On day 42, mice were followed by intraperitoneal injection of UTI89 (1×10⁸ CFU/mouse). The experimental vaccination scheme is shown in FIG. 2A (FIG. 2A). After 16 hours of infection, mice were euthanized, and their kidney, spleen, and liver were collected. The harvested organs were homogenized, and the bacterial burden of UTI89 in infected tissues was evaluated by quantifying colony-forming units (CFU) (FIGS. 2B-2C).

Combining the counts from all cohorts (to assess the total effect of vaccination across all organs), mice immunized with pro-HlyA exhibited a significant reduction in bacterial burden across all organs (Adjusted P-value, P<0.0001) (FIG. 2B). Compared to control group mice, pro-HlyA immunized mice showed a 1.14-log reduction in median UTI89 level at 16 hours post-infection. Additionally, pro-HlyA vaccination led to significant reduction in bacterial levels within each organ type; 1.65-log, 1.12-log, and 1.19-log reductions, in median UTI89 levels in the kidneys, spleen, and liver, respectively (Adjusted P-value, kidney, P=0.0006; spleen, P=0.0008; liver, P<0.0001), when compared to control group mice (FIG. 2C).

### Example 6

### Pro-HlyA Immunization and Lethal Disease

The long-term survival rate following pro-Hly A immunization and challenge with a lethal dose of ExPEC was assessed. The experimental group mice received three subcutaneous immunizations of pro-HlyA on day 0, 14, and 28, while mice in the control group remained unvaccinated (alum only injection). On day 42, mice were intraperitoneally injected with UTI89 (5×10⁷ CFU/mouse) and monitored twice daily for morbidity and mortality over the next 10 days. If no vaccinated mice died of infection, a small number surviving vaccinated mice were randomly selected and euthanized at 3 days post-infection (−3 d.p.i group) to assess changes in bacterial burden within infected organs over time. At 10 d.p.i, all remaining surviving vaccinated mice were euthanized. The harvested organs were homogenized, and UTI89 bacterial burden within infected tissues was quantified by determining CFU. The vaccination scheme used in this study is shown in FIG. 2D (FIG. 2D).

Our results indicated that unvaccinated mice died within 1 d.p.i, whereas 4 surviving pro-HlyA vaccinated mice were euthanized at 3 d.p.i during the 10-day observation period (−3 d.p.i group), and all the remaining 8 Dual-Hit vaccinated mice survived until the end of the observation period (−10 d.p.i group), resulting in a 66.7% survival rate at 10 d.p.i (Adjusted P-value, P<0.0001) (FIG. 2E). The bacterial burden results in pro-HlyA vaccinated mice correlated with survival rates. Combining the counts from all organs, pro-HlyA immunized mice exhibited a significant reduction in UTI89 bacterial burden at both 3 d.p.i (Adjusted P-value, P=0.0060) and 10 d.p.i (Adjusted P-value, P<0.0001), compared to unvaccinated mice. Moreover, in comparison to unvaccinated mice, the pro-HlyA vaccination resulted in approximately 5.42-log and 8.57-log reductions in median UTI89 bacterial burden at 3 d.p.i and 10 d.p.i, respectively (FIG. 2F). Furthermore, pro-HlyA vaccinated mice surviving for 10 d.p.i showed a 3.15-log significant reduction in median UTI89 levels (Adjusted P-value, P=0.0147) compared to those euthanized at 3 d.p.i, suggesting enduring and consistent protection against UTI89 infection conferred by pro-HlyA immunization. Additionally, combining the counts at both 3 d.p.i and 10 d.p.i and analyzing by each organ, pro-HlyA immunized mice demonstrated a significant decrease in bacterial loads across multiple organs compared to unvaccinated mice (Adjusted P-value, kidney, P=0.0013; spleen, P=0.0040; liver, P<0.0001) (FIG. 2G).

### Example 7

### Efficacy of a Combined Auto-Transporter-Toxin Vaccine for ExPEC

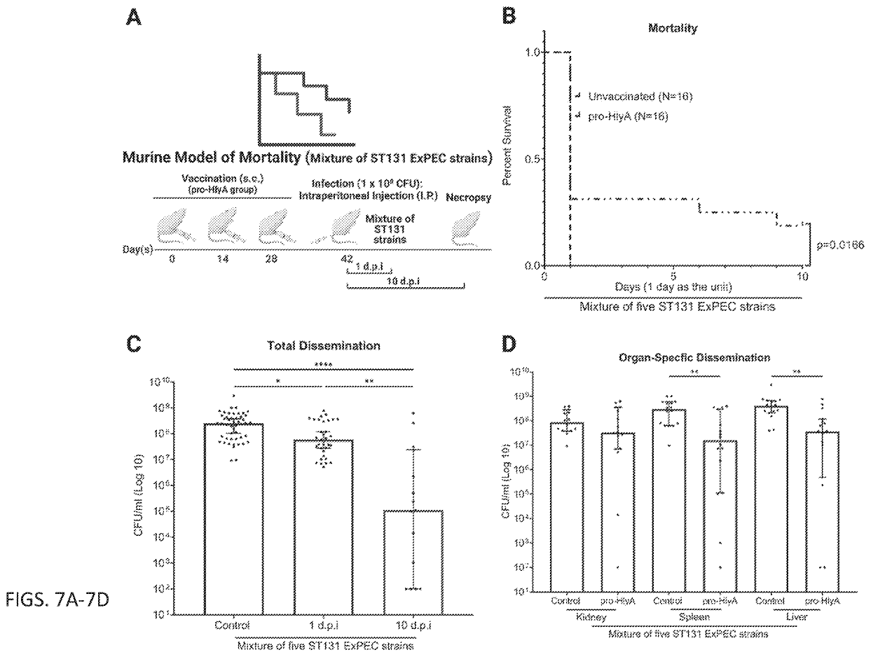

In specific embodiments, vaccination with pro-HlyA is protective against ExPEC that encodes the hemolysin, but it will not provide sufficient protection at least under certain conditions against ST131 ExPEC strains that is likely virulent but does not require HlyA for its virulence (FIG. 7). Whereas such a finding may demonstrate the specificity of the results observed in FIG. 2 towards HlyA, in some embodiments it may mean that strains that lack this antigen will still cause substantial disease even if the recipient is vaccinated. However, earlier research also demonstrated that SinH-3, a fragment corresponding to the immunoglobulin-like (Ig-like) domain-3 of the invasin-like autotransporter protein SinH, conferred robust systemic protection against infections caused by ST131 ExPEC strains in multiple murine models [77]. Given the insufficient protection induced by pro-HlyA immunization alone against a mixture of ST131 ExPEC strains that lack the hlyA sequence (including ST131-H30R lineage) (FIG. 7), the combination vaccine comprising SinH-3 and pro-HlyA (hereafter referred to as “Dual-Hit”) against several sequence types of ExPEC strains was characterized. It was assessed whether Dual-Hit still maintained robust protective efficacy against representative ExPEC strains containing hlyA sequences, such as UTI89, in both bacteremia and mortality models.

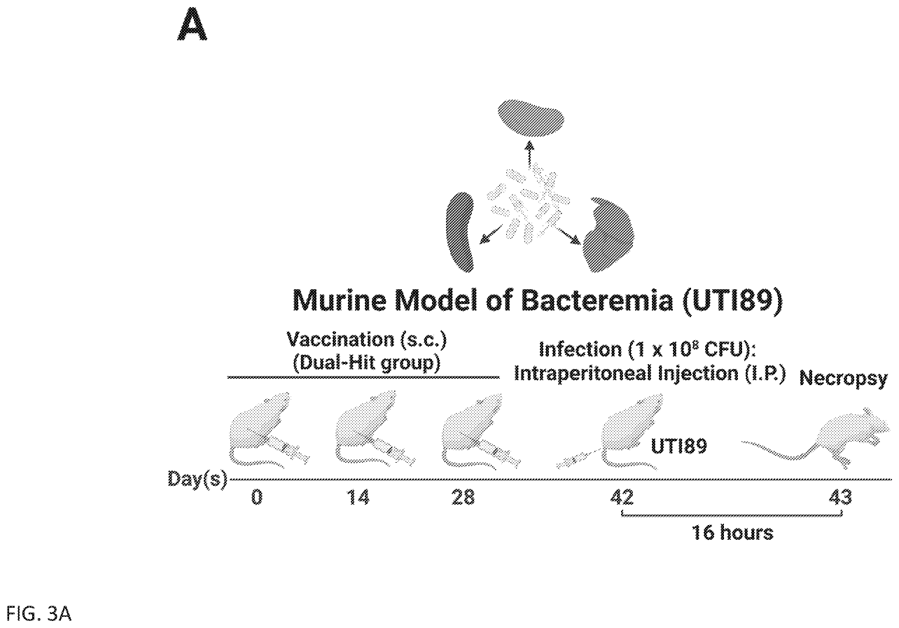

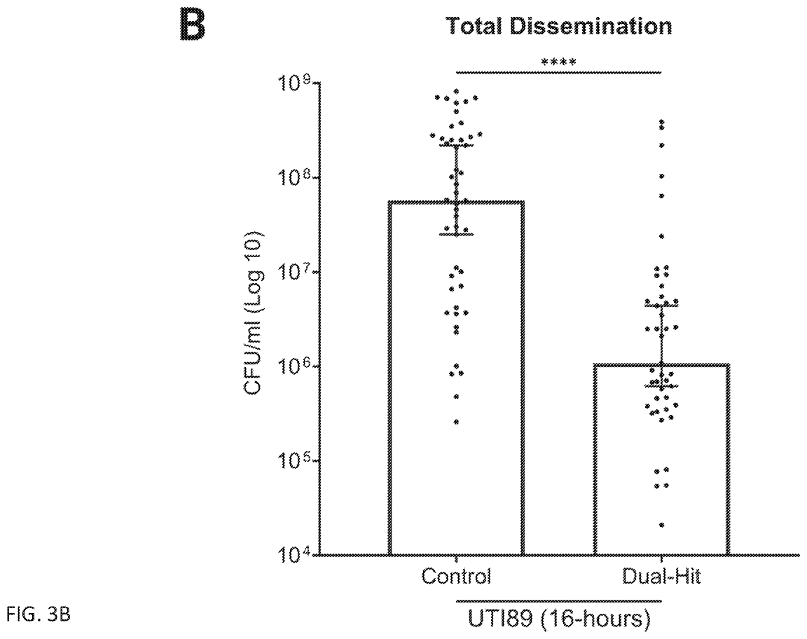

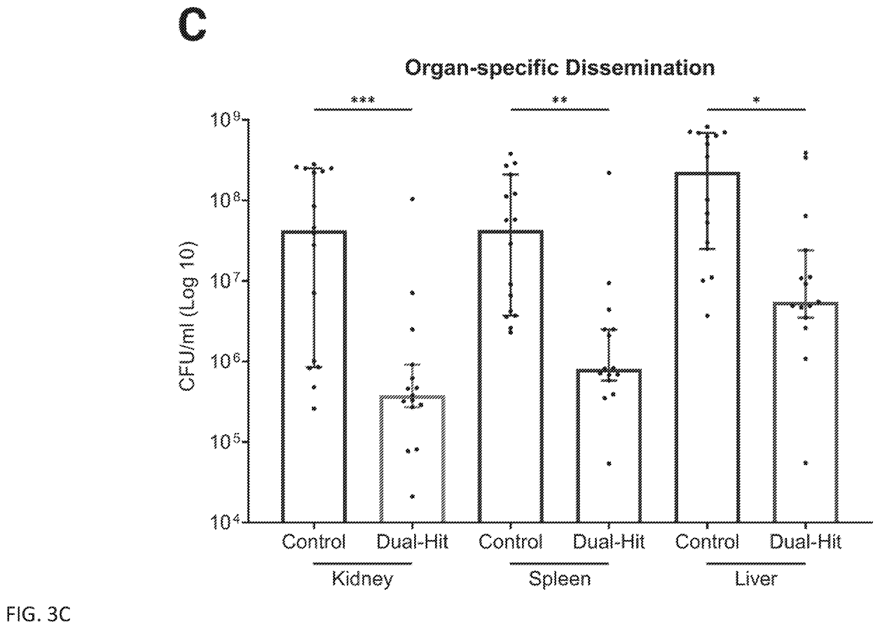

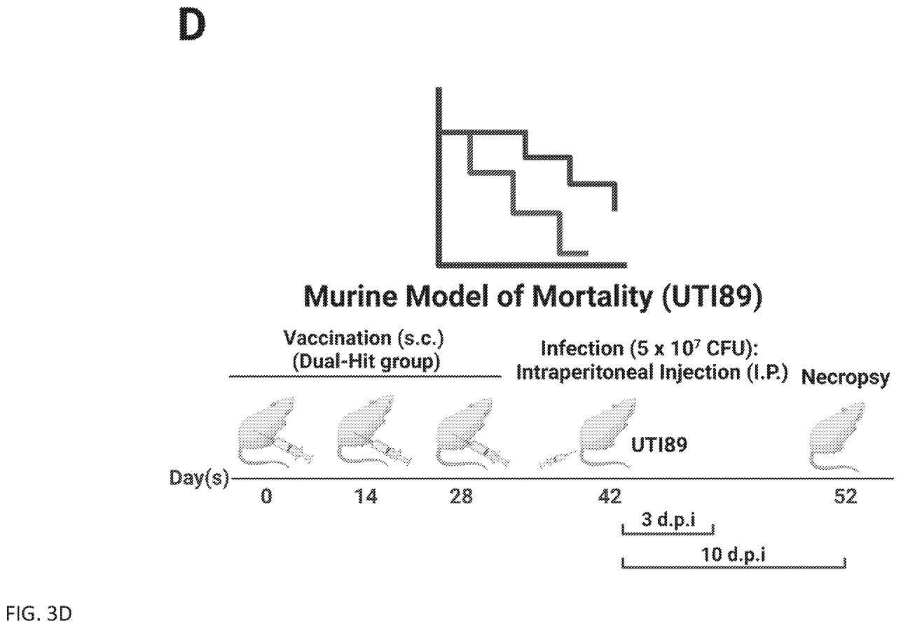

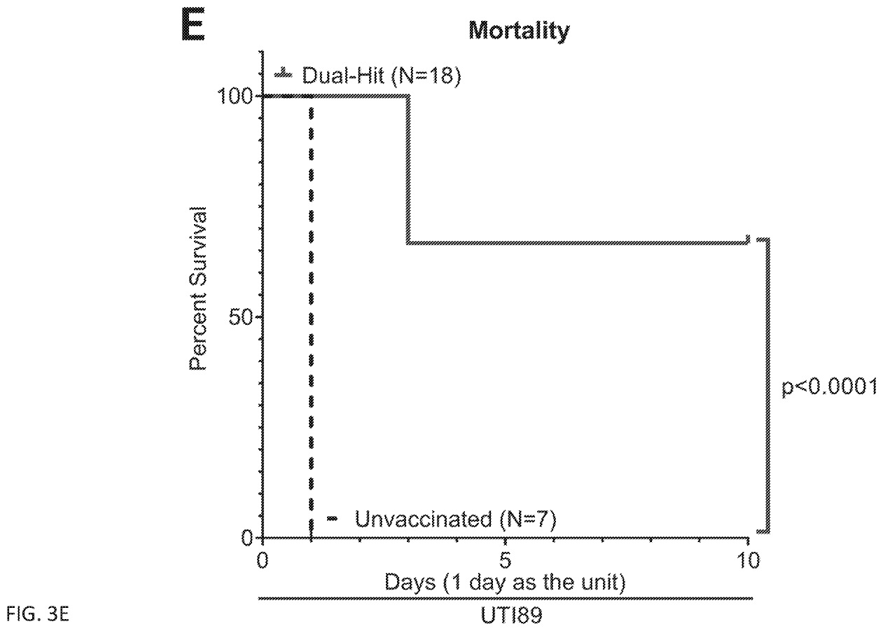

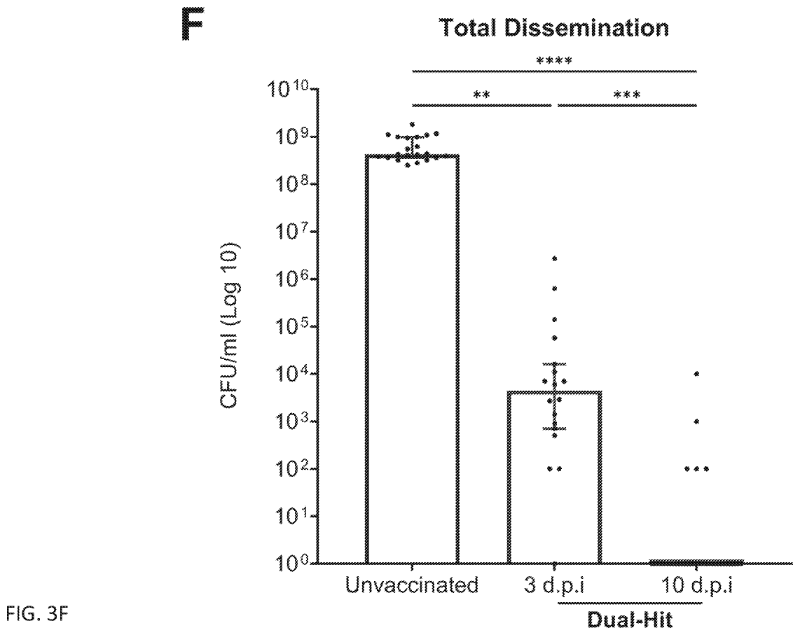

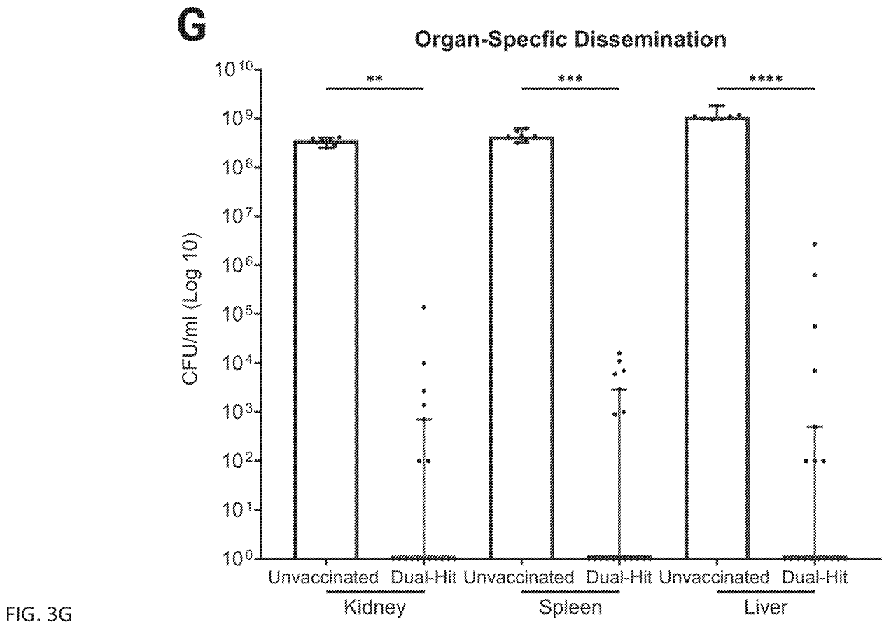

To evaluate the rapid protective efficacy of Dual-Hit immunization in a UTI89 bacteremia model, the experiment and control group mice were immunized and challenged as described in FIG. 2. The experimental vaccination scheme is shown in FIG. 3A (FIG. 3A). After 16 hours of infection, mice were euthanized simultaneously, their organs were harvested and homogenized, and the bacterial burden of UTI89 was quantified by measuring CFU (FIGS. 3B-3C). Combining the counts from all cohorts, Dual-Hit vaccinated mice demonstrated a significant reduction in bacterial burden across all organs (Adjusted P-value, P<0.0001) (FIG. 3B). Compared to the control group mice, Dual-Hit immunized mice exhibited an approximately 1.73-log reduction in median UTI89 level at 16 hours post-infection, indicating robust and rapid protection across multiple organs. Moreover, Dual-Hit vaccination resulted in significant reductions in bacterial levels within each collected organ. Compared to control group mice, those vaccinated with Dual-Hit exhibited approximately 2.05-log, 1.72-log, and 1.61-log reductions in median UTI89 levels in the kidneys, spleen, and liver, respectively (Adjusted P-value, kidney, P=0.0009; spleen, P=0.0047; liver, P=0.0289) (FIG. 3C). These findings indicated that Dual-Hit immunization provides rapid, systemic protection against UTI89 bacteremia across multiple organs within a brief timeframe.

### Example 8

### Dual-Hit and Lethal Bacteremia

It was investigated whether subcutaneous immunization with Dual-Hit increased the survival rate of vaccinated mice when challenged with UTI89 using the same conditions as described in FIG. 2 (FIG. 3D). Our results showed that unvaccinated mice died within 1 d.p.i, whereas 6 surviving Dual-Hit vaccinated mice were euthanized at 3 d.p.i during the 10-day observation period (−3 d.p.i group), and all the remaining 12 Dual-Hit vaccinated mice survived until the end of the observation period (−10 d.p.i group), resulting in a survival rate of 66.7% at 10 d.p.i (adjusted P-value, P<0.0001) (FIG. 3E). Bacterial burden measurements in Dual-Hit vaccinated mice correlated with survival rates. When combining the counts from all organs, Dual-Hit immunized mice showed a significant reduction in UTI89 bacterial burdens at both 3 d.p.i (Adjusted P-value, P=0.0037) and 10 d.p.i (Adjusted P-value, P<0.0001), compared to unvaccinated mice. Moreover, in comparison to unvaccinated mice, the median UTI89 bacterial burden in Dual-Hit vaccinated mice was approximately 4.99-log and 8.63-log lower at 3 d.p.i and 10 d.p.i, respectively (FIG. 3F). Additionally, Dual-Hit vaccinated mice surviving for 10 d.p.i showed a 3.65-log significant reduction in median UTI89 levels (Adjusted P-value, P=0.0003) compared to those euthanized at 3 d.p.i. When combining bacterial burden counts at both 3 d.p.i and 10 d.p.i and analyzing by each organ, Dual-Hit immunized mice demonstrated significant reductions in bacterial burdens across multiple organs compared to unvaccinated mice (Adjusted P-value, kidney, P=0.0013; spleen, P=0.0003; liver, P<0.0001) (FIG. 3G).

### Example 9

### Pro-HLYA or Dual-Hit and Protection Against Prototype Strain CFT073

CFT073, a prototypical UPEC strain isolated from a female patient with acute pyelonephritis, belongs to phylogenetic group B2 and multilocus sequence type 73 (ST73) [113,114]. Notably, ST73 represents one of the most prevalent UPEC lineages, accounting for 11% and 16.6% of UPEC isolates obtained from UTI patients (including the elderly) in recent studies [115,116]. It was next investigated whether immunization with pro-HlyA or Dual-Hit confers robust protection against CFT073 in the murine model of mortality.

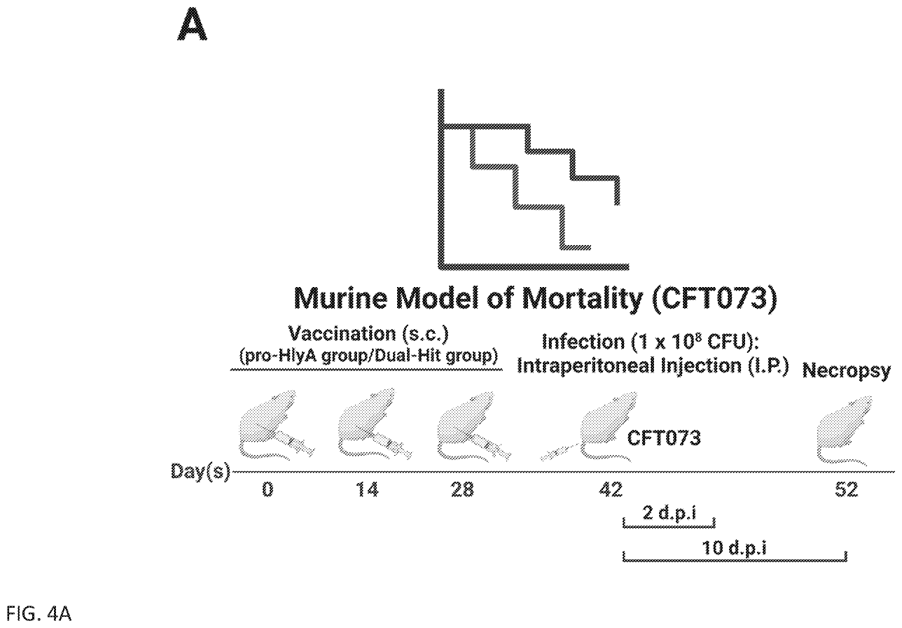

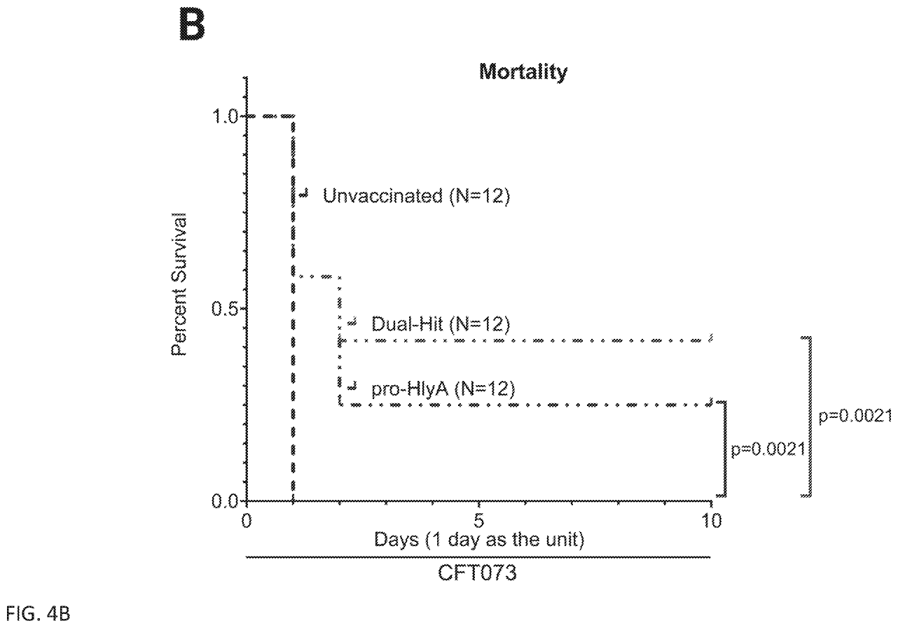

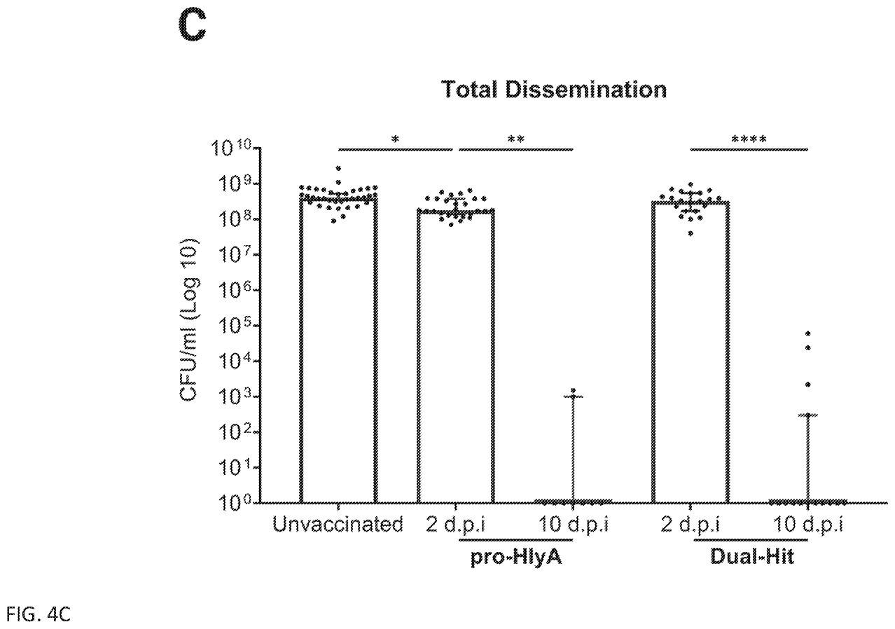

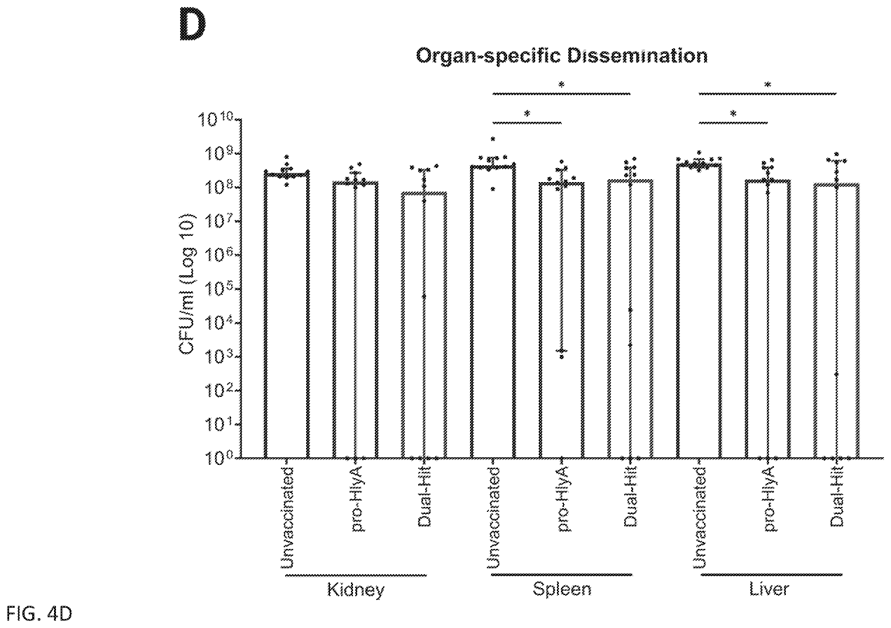

The vaccination was as described above with the scheme shown in FIG. 4A (FIG. 4A). The results indicated that all unvaccinated mice died within 1 d.p.i. whereas mice immunized with pro-HlyA exhibited a 25% survival rate at 10 d.p.i (adjusted P-value, P=0.0021), while Dual-Hit vaccinated mice showed a 42% survival rate at 10 d.p.i (adjusted P-value, P=0.0021) (FIG. 4B). Although some vaccinated mice died within 2 d.p.i, immunization with pro-HlyA or Dual-Hit significantly improved survival rates after CFT073 infections. Bacterial loads in both pro-HlyA and Dual-Hit immunized mice correlated with survival rates. When combining counts from all organs, compared to unvaccinated mice, pro-HlyA immunized mice demonstrated a statistically significant reduction in CFT073 bacterial burden at both 2 d.p.i (Adjusted P-value, P=0.0184) and 10 d.p.i (Adjusted P-value, P<0.0001). Surviving pro-HlyA vaccinated mice exhibit an approximate 8.61-log reduction in the median level of CFT073 at 10 d.p.i relative to unvaccinated mice that died within 1 d.p.i. Additionally, pro-HlyA vaccinated mice surviving at 10 d.p.i showed a significant reduction in the median level of CFT073 compared to those moribund or died within 2 d.p.i (Adjusted P-value, P=0.0035), indicating sustained and persistent protection caused by pro-HlyA immunization against CFT073 infection over time (FIG. 4C). Similarly, surviving Dual-Hit vaccinated mice demonstrated a significant reduction in bacterial burden at 10 d.p.i compared to unvaccinated mice (Adjusted P-value, P<0.0001), with an approximately 8.61-log reduction in the median level of CFT073 strain. However, there is no difference in bacterial burden between the 2 d.p.i Dual-Hit vaccinated group mice and unvaccinated group mice. Moreover, the median CFT073 levels were substantially reduced when comparing Dual-Hit vaccinated mice that survived at 10 d.p.i to those that died within 2 d.p.i (Adjusted P-value, P<0.0001). When combining bacterial burden counts at both 2 d.p.i and 10 d.p.i and analyzing them by each organ, pro-HlyA vaccinated mice exhibited a statistically significant reduction in bacterial burdens in the spleen (Adjusted P-value, P=0.0146) and liver (Adjusted P-value, P=0.0349) compared to the bacterial loads in unvaccinated mice (FIG. 4D). Similarly, Dual-Hit vaccinated mice showed a statistically significant reduction in bacterial levels in the spleen (Adjusted P-value, P=0.0144) and liver (adjusted P-value, P=0.0186) compared to the bacterial burden in unvaccinated mice (FIG. 4D).

### Example 10

### Pro-HLYA or Dual-Hit and Cystitis Caused by UTI89 in the Murine Model of UTI

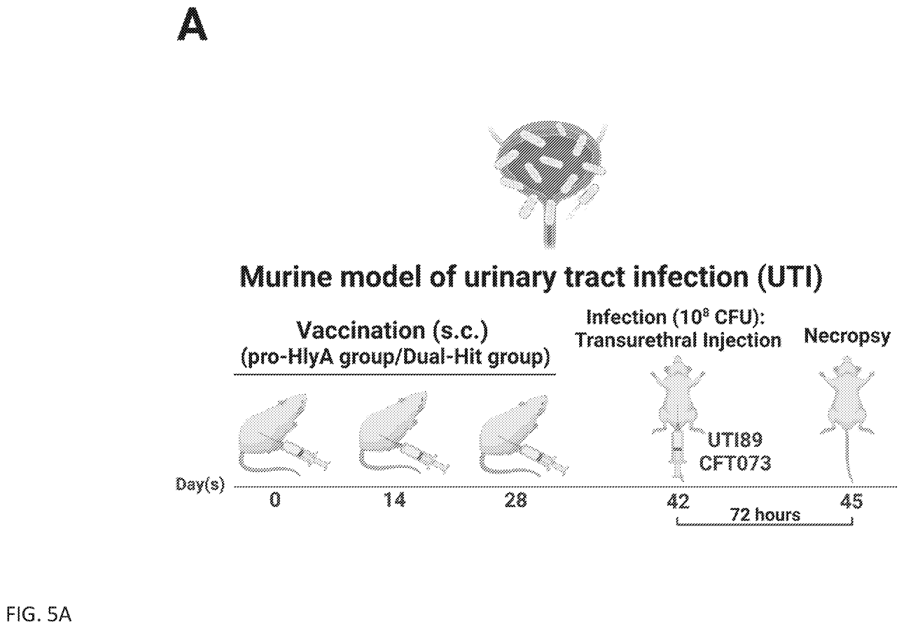

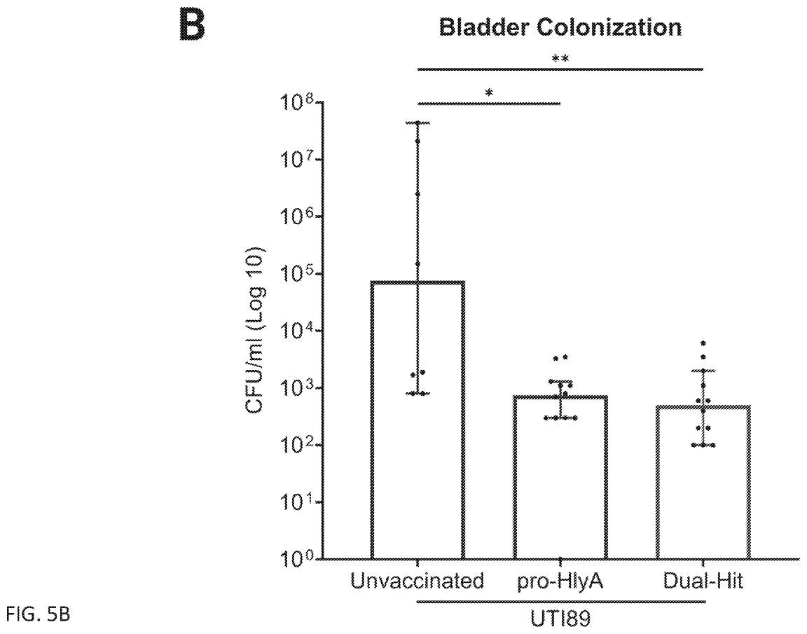

Urinary tract infections (UTIs) constitute a major global health concern, significantly contributing to morbidity in otherwise healthy females, with over 60% experiencing a diagnosis during their lifetime [117]. In the United States, the annual incidence of physician-diagnosed UTIs exceeds 10% for females and 3% for males. UPEC is the primary causative agent, accounting for approximately 80% of UTI cases [118]. Therefore, the protective efficacy was evaluated of pro-HlyA or Dual-Hit against UPEC colonization in the bladder in the murine model of UTI. Female BALB/cJ mice were immunized as previously described in FIG. 2. On day 42, mice were transurethrally inoculated with 1×10⁸ CFU of typical UPEC strains (UTI89 or CFT073, FIG. 5A). After 72 hours of infection, bladders were harvested, homogenized, and bacterial loads of UTI89 and CFT073 were determined by quantifying CFUs.

For the experimental UPEC strain UTI89, the results demonstrated that both pro-HlyA and Dual-Hit immunizations provided robust protection against UTI89 colonization in the bladder within the UTI model. In comparison to unvaccinated mice, the pro-HlyA vaccinated mice exhibited an approximate 2.01-log reduction in the median level of UTI89 in their bladder (Adjusted P-value, P=0.0328). Similarly, the Dual-Hit vaccinated mice showed an approximate 2.18-log reduction in the median level of UTI89 (Adjusted P-value, P=0.0094) (FIG. 5B). However, no significant differences were observed between the experimental groups and the control group for the experimental UPEC strain CFT073 (FIG. 5C).

### Example 11

### Dual-Hit Vaccination and a Mixture of Ten Typical ExPEC Strains

The robust protective efficacy of Dual-Hit against UTI89 (ST95) and CFT073 (ST73) in murine models of bacteremia and mortality. Additionally, immunization with pro-HlyA alone provided inadequate protection against the infections caused by ExPEC ST131 strains that lack the hlyA sequence in the murine mortality model. Consequently, it was evaluated whether Dual-Hit could offer robust protective efficacy and significantly increase survival rates against a mixture of ten typical ExPEC strains (including ST95, ST73, and ST131) in the murine mortality model.

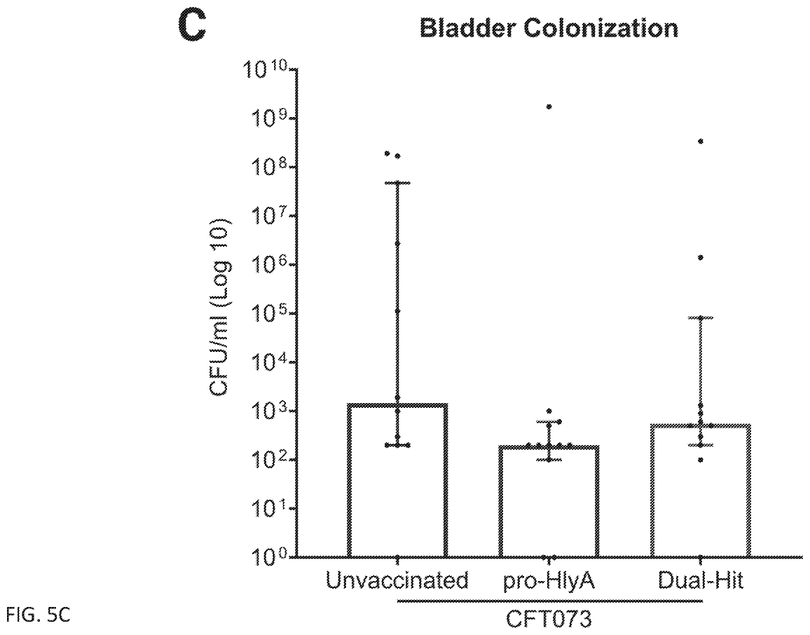

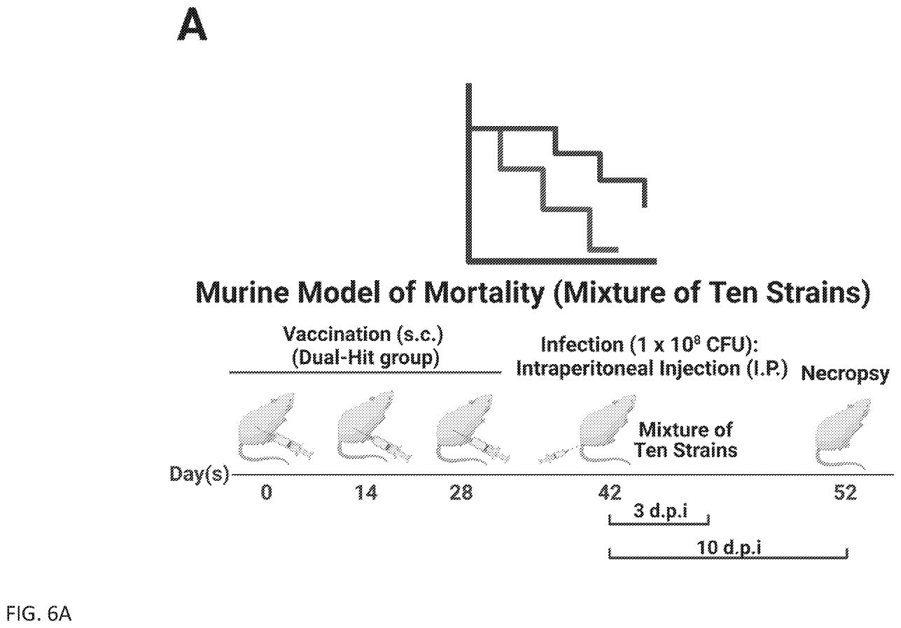

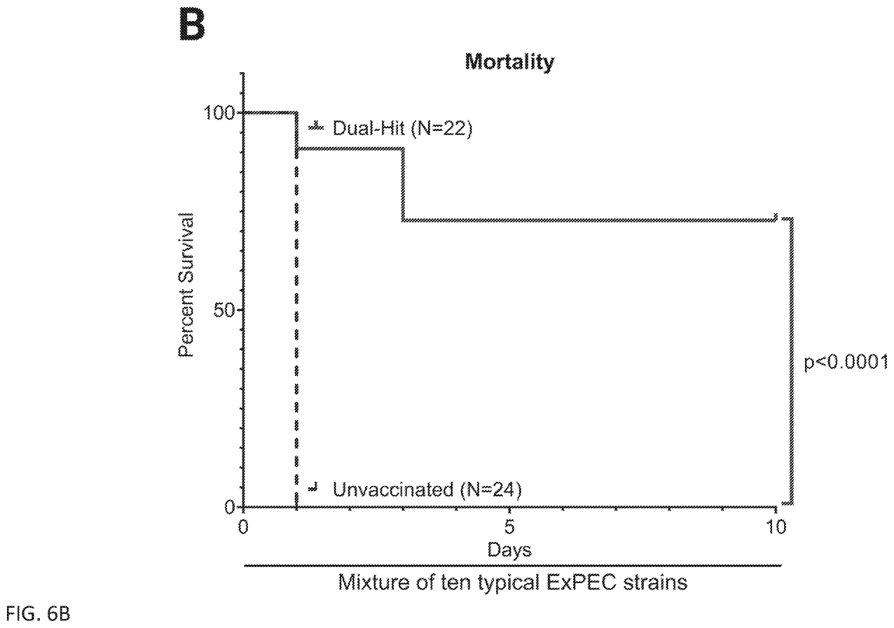

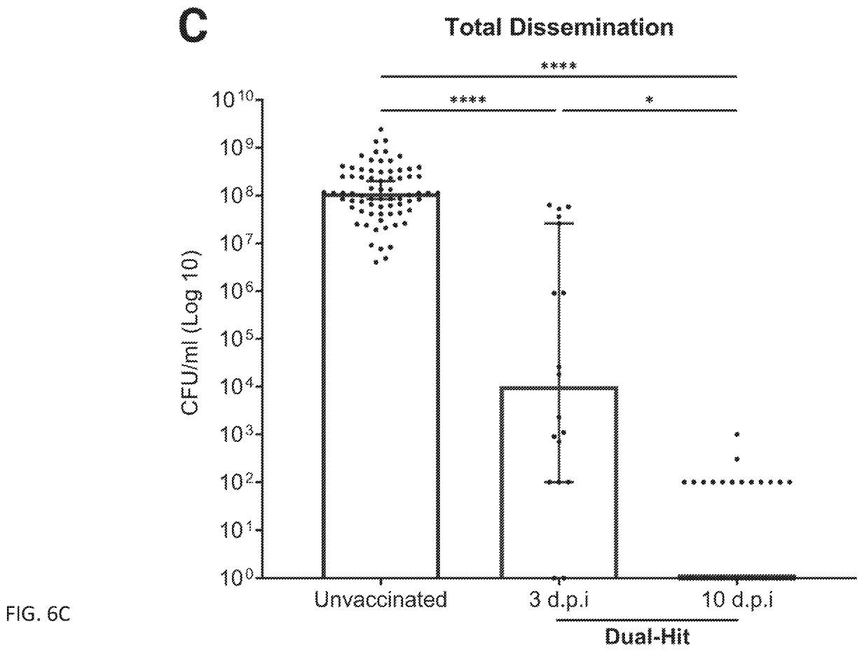

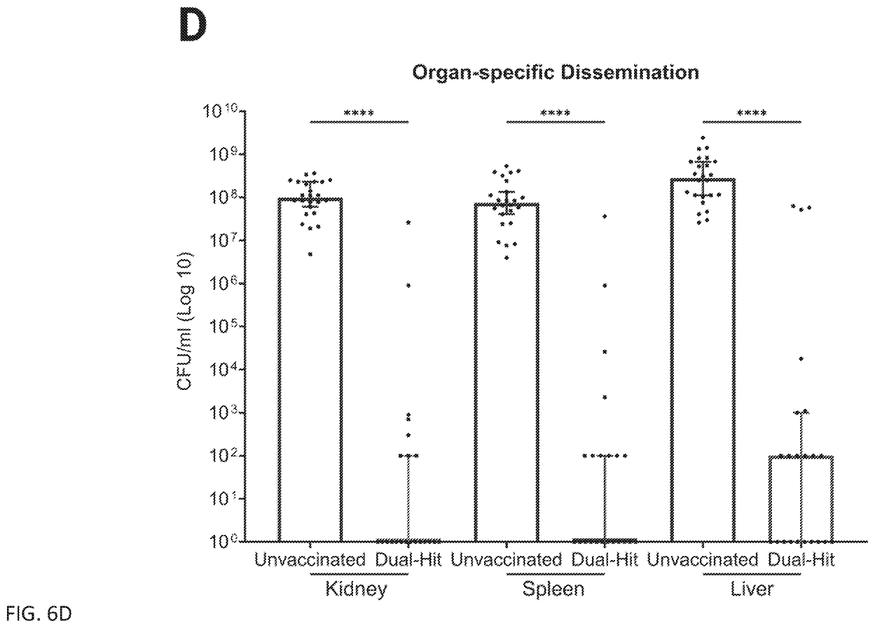

To assess the protective efficacy of the Dual-Hit against multiple sequence types of ExPEC strains in the murine mortality model, experiment group vaccinated as previously described, while a control group remained unvaccinated (FIG. 6A). On day 42, mice were intraperitoneally challenged with a mixture of ten typical ExPEC strains (1×10⁸ CFU/mouse in total), representing a range of common high virulent sequence types ExPEC strains (CFT073 (ST73), UTI89 (ST95), W0008 (ST127), JJ1886, JJ1901, JJ2050, JJ2528, JJ2547 (ST131), W0044 (ST405-like), and W0128 (ST648-like) in equal proportions). Over the next 10 days, mice were closely monitored for morbidity and mortality twice daily.

The findings revealed that unvaccinated mice died within 1 d.p.i, while 2 of the 18 Dual-Hit vaccinated mice died within 1 d.p.i and 4 surviving Dual-Hit vaccinated mice were euthanized at 3 d.p.i during the 10-day observation period (−3 d.p.i group), and all the remaining 16 Dual-Hit vaccinated mice survived until the end of the observation period (−10 d.p.i group), resulting in a survival rate of 72.7% at 10 d.p.i (adjusted P-value, P<0.0001) (FIG. 6B). When combining CFU counts from all organs, mice immunized with Dual-Hit demonstrated significantly reduced bacterial burdens at both 3 d.p.i (Adjusted P-value, P<0.0001) and 10 d.p.i (Adjusted P-value, P<0.0001) compared to unvaccinated mice. Compared to unvaccinated mice, the median bacterial burden in Dual-Hit vaccinated mice was approximately 4.04-log and 8.05-log lower at 3 d.p.i and 10 d.p.i, respectively (FIG. 6C). A 4.01-log reduction in the median level of ten ExPEC strains was also observed when comparing the bacterial levels in Dual-Hit vaccinated mice at 10 d.p.i to those at 3 d.p.i (Adjusted P-value, P=0.0148), suggesting Dual-Hit immunization provided sustained and persistent protection against ExPEC infection over time (FIG. 6C). Furthermore, when combining the counts at both 3 d.p.i and 10 d.p.i and analyzing by each organ, Dual-Hit immunized mice demonstrated a significant reduction in bacterial loads across multiple organs compared to unvaccinated mice (Adjusted P-value, kidney, P<0.0001; spleen, P<0.0001; liver, P<0.0001) (FIG. 6D).

### Example 12

### Significance of Certain Embodiments

ExPEC is the predominant cause of bacteremia and UTIs, persisting in both community environments and among hospitalized patients, leading to considerable hospitalization and mortality rates. The clinical management of ExPEC faces challenges, which are further exacerbated by the overprescription of antibiotics, the emergence of antibiotic-resistant ExPEC strains, and the global aging trend [119-121]. A vaccine targeting ExPEC represents a promising alternative strategy to address this issue, mitigating the escalating global burden of antimicrobial resistance crisis and reducing hospitalization cost, thereby providing tremendous worldwide benefits.

In this disclosure, it was demonstrated that; (i) immunization with pro-HlyA or Dual-Hit elicited a rapid and robust protection against the highly virulent ExPEC strain UTI89 (ST95), reducing the bacterial burden of UTI89 in the murine bacteremia model; (ii) immunization with pro-HlyA or Dual-Hit increased survival rates following UTI89 infection, providing lasting and consistent protection in the murine model of mortality; (iii) immunization with pro-HlyA or Dual-Hit conferred partial protection against the highly virulent ExPEC strain CFT073 (ST73), decreasing the bacterial burden of CFT073 and increasing survival rates after CFT073 infection in the murine model of mortality; (iv) immunization with pro-HlyA or Dual-Hit reduced UTI89-induced cystitis in the murine model of UTI; (v) immunization with Dual-Hit significantly increased survival rates following infection by a mixture of ten typical ExPEC strains in the murine model of mortality, indicating the synergistic and broad-spectrum effects of the two antigens. Overall, the data indicate that both the inactive form of hemolysin, pro-HlyA, and Dual-Hit, a combination of the extracellular domains of the autotransporter SinH (SinH-3) and pro-HlyA, represent useful ExPEC immunogenic compositions, including vaccine. This offers an alternative approach to the current ExPEC vaccine development efforts.

Hemolysin is a prevalent exotoxin produced by E. coli and significantly amplifies virulence in various clinical infections. Despite the relatively low abundance of hlyA in the phylogroup database as a whole, it is concentrated in highly virulent sequence types associated with ExPEC and UPEC infections, indicating it plays a role in these infections (FIG. 1A). Interestingly, the alignment and phylogenetic analysis of HlyA shows that the majority of instances of HlyA in what are generally considered intestine-associated phylogroups (A, B1, E) cluster together (FIG. 1B). In specific embodiments, this indicates one or more of the following three different things: 1) the convergent evolution of a less-virulent (or more specialized) allele of hlyA, 2) a more promiscuous form of the pathogenicity island carrying hlyA, 3) increased horizontal transfer due to a higher likelihood of co-colonization. Given that the alleles found within non-B2 phylogroups are nested within the B2 phylogroup alleles, the results suggest that the B2 phylogroup acts as a reservoir for this virulence factor and that it occasionally spills over into other phylogroups, as we hypothesized previously [75].

Furthermore, while both pro-HlyA and Dual-Hit demonstrated high-efficiency protection against UTI89 in various murine models (bacteremia, mortality, and UTI), their protective efficacy against CFT073 in these models was not as robust as anticipated. The differential immunization protective efficacy against these two strains may stem from differences between UTI89 and CFT073. Although E. coli clones ST95 and ST73 frequently cause bloodstream infections and UTIs, a recent study revealed that UTI89 possesses a greater total number of genes that contribute to growth in urine and bladder colonization than CFT073. However, CFT073 appears to have more fitness factors than UTI89 [122,123]. Another study demonstrated that, while both UTI89 and CFT073 are clinical UPEC isolates that could cause infections for at least two weeks in similar proportions of mice, UTI89 infections could persist indefinitely, compared to the CFT073 infections began to clear two weeks after inoculation [124]. These findings suggest that UTI89 might express more virulence factors on the bacterial surface compared to CFT073, leading to more persistent infections but also increasing detectability and bind-ability by vaccine-specific antibodies against UTI89, leading to a higher protective efficacy of pro-HlyA and Dual-Hit immunization.

Variations in immunization routes and adjuvant types significantly impact vaccine efficacy evaluation. Understanding these would enable optimization of the vaccine formulation and administration to maximize protective efficacy. For instance, intramuscular (I.M.) administration is the most used route for licensed vaccines and has been shown to elicit high immunogenicity in adult rabbits immunized with MecVax, producing antibodies against enterotoxigenic Escherichia coli (ETEC) H10407 and reducing intestinal colonization [125]. A clinical trial also demonstrated the safety and immunogenicity of the CS6-targeted candidate vaccine, CssBA, when administered intramuscularly [126]. Furthermore, both experimental and clinical evidence have indicated that mucosal immunization could efficiently induce local and distant systemic immune responses, as well as in the blood [127,128]. In previous studies, mice intranasally immunized with the iron receptor, FyuA, IutA, Hma, and IreA exhibited a robust and long-lived humoral immune response against UPEC challenge. Intranasal immunization with FyuA reduced UPEC strain 536 colonization following transurethral challenge, while IreA intranasally immunization significantly reduced CFT073 bacterial counts in the bladder [45,46]. Therefore, without compromising the robust systemic protection provided by pro-HlyA or Dual-Hit immunization, combining subcutaneous with either intramuscular or intranasal routes may present a more promising approach to enhance immune responses and improve protective efficacy of vaccinated mice against ExPEC infections in both blood (bacteremia) and mucosal (urinary tract).

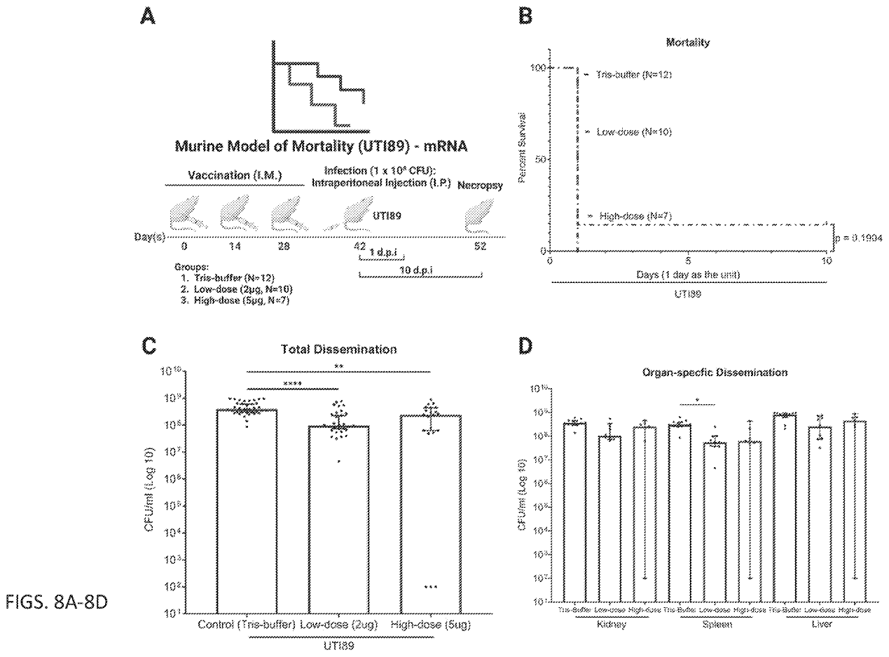

The use of a vaccine could also be significantly enhanced by formulating it with novel adjuvants, which effectively augment immune responses to the administered vaccine antigen [129]. In the study, aluminum salts (alum) were utilized as the adjuvant due to its proven safety. Alum is a clinically approved and widely used adjuvant in human vaccines, has been used for over 80 years in vaccine research and typically stimulates the The2-type immune responses [130]. In a previous study, suitable adjuvants were screened for iron receptor-based immunization against UPEC infection, and they found that dmLT generated the most consistently robust antibody response in intranasally immunized mice, while Monophosphoryl-Lipid A (MPLA) and alum produced greater concentrations of antigen-specific IgG with intramuscular immunization [131]. This study indicates that dmLT, a mucosal adjuvant proven safe and potent through both preclinical and clinical studies, is a useful adjuvant in some embodiments. Similarity, a recent study demonstrated that following bladder infection, highly T-helper type 2 (TH2)-skewed immune responses prioritized bladder epithelial repair after extensive exfoliation of epithelial cells, rather than bacterial clearance and even inhibition of Th1-mediated responses [132]. Therefore, MPLA is a potentially ideal adjuvant that could safely induce an appropriate level of Th1 response, enhancing Th1-mediated bacteria-clearing responses and increasing the ability to eliminate E. coli infection after vaccine immunization [133]. Of note, a mRNA version was generated of the Dual-Hit vaccine described here and tested for efficacy against UTI89. However, in specific embodiments there was no difference in survival or protection between vaccinated and control groups under these conditions (FIG. 8).

In summary, antimicrobial resistance is a leading threat to global health. Developing an ExPEC vaccine presents a strategy to combat this growing global crisis and effectively reduce the incidence of antibiotic-resistant ExPEC infections to improve public health outcomes. The study demonstrates the promising protective efficacy of pro-HlyA and Dual-Hit immunizations against ExPEC infections in murine models. The observed reduction in bacterial burden and increased survival rates indicate the use of these vaccines in the clinic. Furthermore, in this disclosure, there was a bridge between computational genomics and virulome vaccinology, and blocking of various steps of bacterial pathogenesis by synergizing multiple protein subunits associated with different virulence factors.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Poolman J T, Wacker M. Extraintestinal Pathogenic Escherichia coli, a Common Human Pathogen: Challenges for Vaccine Development and Progress in the Field. J Infect Dis. 2016 Jan. 1; 213 (1): 6-13. doi: 10.1093/infdis/jiv429. Epub 2015 Sep. 2. PMID: 26333944; PMCID: PMC4676548.
2. Antimicrobial Resistance Collaborators. Global burden of bacterial antimicrobial resistance in 2019: a systematic analysis. Lancet. 2022 Feb. 12; 399 (10325): 629-655. doi: 10.1016/S0140-6736 (21) 02724-0. Epub 2022 Jan. 19. Erratum in: Lancet. 2022 Oct. 1; 400 (10358): 1102. PMID: 35065702; PMCID: PMC8841637.
3. Sora V M, Meroni G, Martino P A, Soggiu A, Bonizzi L, Zecconi A. Extraintestinal Pathogenic Escherichia coli: Virulence Factors and Antibiotic Resistance. Pathogens. 2021 Oct. 20; 10 (11): 1355. doi: 10.3390/pathogens10111355. PMID: 34832511; PMCID: PMC8618662.
4. Russo T A, Johnson J R. Medical and economic impact of extraintestinal infections due to Escherichia coli: focus on an increasingly important endemic problem. Microbes Infect. 2003 April; 5 (5): 449-56. doi: 10.1016/s1286-4579 (O3) 00049-2. PMID: 12738001.
5. Ku L C, Boggess K A, Cohen-Wolkowiez M. Bacterial meningitis in infants. Clin Perinatol. 2015 March; 42 (1): 29-45, vii-viii. doi: 10.1016/j.clp.2014.10.004. Epub 2014 Dec. 6. PMID: 25677995; PMCID: PMC4332563.
6. Angus D C, Linde-Zwirble W T, Lidicker J, Clermont G, Carcillo J, Pinsky M R. Epidemiology of severe sepsis in the United States: analysis of incidence, outcome, and associated costs of care. Crit Care Med. 2001 July; 29 (7): 1303-10. doi: 10.1097/00003246-200107000-00002. PMID: 11445675.
7. Liu V, Escobar G J, Greene J D, Soule J, Whippy A, Angus D C, Iwashyna T J. Hospital deaths in patients with sepsis from 2 independent cohorts. JAMA. 2014 Jul. 2; 312 (1): 90-2. doi: 10.1001/jama.2014.5804. PMID: 24838355.
8. Epstein L, Dantes R, Magill S, Fiore A. Varying Estimates of Sepsis Mortality Using Death Certificates and Administrative Codes—United States, 1999-2014. MMWR Morb Mortal Wkly Rep. 2016 Apr. 8; 65 (13): 342-5. doi: 10.15585/mmwr.mm6513a2. PMID: 27054476.
9. Rudd K E, Johnson S C, Agesa K M, Shackelford K A, Tsoi D, Kievlan D R, Colombara D V, Ikuta K S, Kissoon N, Finfer S, Fleischmann-Struzek C, Machado F R, Reinhart K K, Rowan K, Seymour C W, Watson R S, West T E, Marinho F, Hay S I, Lozano R, Lopez A D, Angus D C, Murray C J L, Naghavi M. Global, regional, and national sepsis incidence and mortality, 1990-2017: analysis for the Global Burden of Disease Study. Lancet. 2020 Jan. 18; 395 (10219): 200-211. doi: 10.1016/S0140-6736 (19) 32989-7. PMID: 31954465; PMCID: PMC6970225.
10. Paoli C J, Reynolds M A, Sinha M, Gitlin M, Crouser E. Epidemiology and Costs of Sepsis in the United States—An Analysis Based on Timing of Diagnosis and Severity Level. Crit Care Med. 2018 December; 46 (12): 1889-1897. doi: 10.1097/CCM.0000000000003342. PMID: 30048332; PMCID: PMC6250243.
11. Bonten M, Johnson J R, van den Biggelaar A H J, Georgalis L, Geurtsen J, de Palacios P I, Gravenstein S, Verstraeten T, Hermans P, Poolman J T. Epidemiology of Escherichia coli Bacteremia: A Systematic Literature Review. Clin Infect Dis. 2021 Apr. 8; 72 (7): 1211-1219. doi: 10.1093/cid/ciaa210. PMID: 32406495.
12. Dadgostar P. Antimicrobial Resistance: Implications and Costs. Infect Drug Resist. 2019 Dec. 20; 12:3903-3910. doi: 10.2147/IDR.S234610. PMID: 31908502; PMCID: PMC6929930.
13. Ohmagari N, Choi W S, Tang H J, Atanasov P, Jiang X, Hernandez Pastor L, Nakayama Y, Chiang J, Lim K, Nievera M C. Targeted literature review of the burden of extraintestinal pathogenic Escherichia Coli among elderly patients in Asia Pacific regions. J Med Econ. 2023 January-December; 26 (1): 168-178. doi: 10.1080/13696998.2023.2169447. PMID: 36647596.
14. Al-Hasan M N, Razonable R R, Eckel-Passow J E, Baddour L M. Incidence rate and outcome of Gram-negative bloodstream infection in solid organ transplant recipients. Am J Transplant. 2009 April; 9 (4): 835-43. doi: 10.1111/j.1600-6143.2009.02559.x. PMID: 19344469.
15. Qiao B, Wu J, Wan Q, Zhang S, Ye Q. Factors influencing mortality in abdominal solid organ transplant recipients with multidrug-resistant gram-negative bacteremia. BMC Infect Dis. 2017 Feb. 27; 17 (1): 171. doi: 10.1186/s12879-017-2276-1. PMID: 28241746; PMCID: PMC5327527.
16. Pitout J D, DeVinney R. Escherichia coli ST131: a multidrug-resistant clone primed for global domination. F1000Res. 2017 Feb. 28; 6: F1000 Faculty Rev-195. doi: 10.12688/f1000research.10609.1. PMID: 28344773; PMCID: PMC5333602.
17. Nicolas-Chanoine M H, Bertrand X, Madec J Y. Escherichia coli ST131, an intriguing clonal group. Clin Microbiol Rev. 2014 July; 27 (3): 543-74. doi: 10.1128/CMR.00125-13. PMID: 24982321; PMCID: PMC4135899.
18. Shaik S, Ranjan A, Tiwari S K, Hussain A, Nandanwar N, Kumar N, Jadhav S, Semmler T, Baddam R, Islam M A, Alam M, Wieler L H, Watanabe H, Ahmed N. Comparative Genomic Analysis of Globally Dominant ST131 Clone with Other Epidemiologically Successful Extraintestinal Pathogenic Escherichia coli (ExPEC) Lineages. mBio. 2017 Oct. 24; 8 (5): e01596-17. doi: 10.1128/mBio.01596-17. PMID: 29066550; PMCID: PMC5654935.
19. Johnson J R, Johnston B, Clabots C, Kuskowski M A, Castanheira M. Escherichia coli sequence type ST131 as the major cause of serious multidrug-resistant E. coli infections in the United States. Clin Infect Dis. 2010 Aug. 1; 51 (3): 286-94. doi: 10.1086/653932. PMID: 20572763.
20. Tchesnokova V, Riddell K, Scholes D, Johnson J R, Sokurenko E V. The Uropathogenic Escherichia coli Subclone Sequence Type 131-H30 Is Responsible for Most Antibiotic Prescription Errors at an Urgent Care Clinic. Clin Infect Dis. 2019 Feb. 15; 68 (5): 781-787. doi: 10.1093/cid/ciy523. PMID: 29961840; PMCID: PMC6376094.
21. Johnson J R, Porter S, Thuras P, Castanheira M. Epidemic Emergence in the United States of Escherichia coli Sequence Type 131-H30 (ST131-H30), 2000 to 2009. Antimicrob Agents Chemother. 2017 Jul. 25; 61 (8): e00732-17. doi: 10.1128/AAC.00732-17. PMID: 28533233; PMCID: PMC5527581.
22. Xia F, Cheng J, Jiang M, Wang Z, Wen Z, Wang M, Ren J, Zhuge X. Genomics Analysis to Identify Multiple Genetic Determinants That Drive the Global Transmission of the Pandemic ST95 Lineage of Extraintestinal Pathogenic Escherichia coli (ExPEC). Pathogens. 2022 Dec. 7; 11 (12): 1489. doi: 10.3390/pathogens11121489. PMID: 36558824; PMCID: PMC9781279.
23. Riley L W. Pandemic lineages of extraintestinal pathogenic Escherichia coli. Clin Microbiol Infect. 2014 May; 20 (5): 380-90. doi: 10.1111/1469-0691.12646. PMID: 24766445.
24. Wu J, Bao C, Reinhardt R L, Abraham S N. Local induction of bladder Th1 responses to combat urinary tract infections. Proc Natl Acad Sci USA. 2021 Mar. 9; 118 (10):e2026461118. doi: 10.1073/pnas.2026461118. PMID: 33653961; PMCID: PMC7958421.
25. Luzuriaga M A, Herbert F C, Brohlin O R, Gadhvi J, Howlett T, Shahrivarkevishahi A, Wijesundara Y H, Venkitapathi S, Veera K, Ehrman R, Benjamin C E, Popal S, Burton M D, Ingersoll M A, De Nisco N J, Gassensmith J J. Metal-Organic Framework Encapsulated Whole-Cell Vaccines Enhance Humoral Immunity against Bacterial Infection. ACS Nano. 2021 Nov. 23; 15 (11): 17426-17438. doi: 10.1021/acsnano.1c03092. Epub 2021 Sep. 21. PMID: 34546723.
26. Billips B K, Yaggie R E, Cashy J P, Schaeffer A J, Klumpp D J. A live-attenuated vaccine for the treatment of urinary tract infection by uropathogenic Escherichia coli. J Infect Dis. 2009 Jul. 15; 200 (2): 263-72. doi: 10.1086/599839. PMID: 19522648.
27. Russo T A, Beanan J M, Olson R, Genagon S A, MacDonald U, Cope J J, Davidson B A, Johnston B, Johnson J R. A killed, genetically engineered derivative of a wild-type extraintestinal pathogenic E. coli strain is a vaccine candidate. Vaccine. 2007 May 10; 25 (19): 3859-70. doi: 10.1016/j.vaccine.2007.01.100. Epub 2007 Feb. 5. PMID: 17306426; PMCID: PMC1913199.
28. Kochiashvili D, Khuskivadze A, Kochiashvili G, Koberidze G, Kvakhajelidze V. Role of the bacterial vaccine Solco-Urovac® in treatment and prevention of recurrent urinary tract infections of bacterial origin. Georgian Med News. 2014 June; (231): 11-6. PMID: 25020163.
29. Wade D, Cooper J, Derry F, Taylor J. Uro-Vaxom® versus placebo for the prevention of recurrent symptomatic urinary tract infections in participants with chronic neurogenic bladder dysfunction: a randomised controlled feasibility study. Trials. 2019 Apr. 16; 20 (1): 223. doi: 10.1186/s13063-019-3275-x. PMID: 30992071; PMCID: PMC6469220.
30. Brodie A, El-Taji O, Jour I, Foley C, Hanbury D. A Retrospective Study of Immunotherapy Treatment with Uro-Vaxom (OM-89®) for Prophylaxis of Recurrent Urinary Tract Infections. Curr Urol. 2020 October; 14 (3): 130-134. doi: 10.1159/000499248. Epub 2020 Oct. 13. PMID: 33224005; PMCID: PMC7659410. Altschul S F, Gish W, Miller W, Myers E W, Lipman D J. Basic local alignment search tool. J Mol Biol. 1990 Oct. 5; 215 (3): 403-10. doi: 10.1016/S0022-2836 (05) 80360-2. PMID: 2231712.
31. Chorro L, Li Z, Chu L, Singh S, Gu J, Kim J H, Dutta K, Pan R, Kodali S, Ndreu D, Patel A, Hawkins J C, Ponce C, Silmon de Monerri N, Keeney D, Illenberger A, Jones C H, Andrew L, Lotvin J, Prasad A K, Kanevsky I, Jansen K U, Anderson A S, Donald R G K. Preclinical Immunogenicity and Efficacy of Optimized O25b O-Antigen Glycoconjugates To Prevent MDR ST131 E. coli Infections. Infect Immun. 2022 Apr. 21; 90 (4):e0002222. doi: 10.1128/iai.00022-22. Epub 2022 Mar. 21. PMID: 35311580; PMCID: PMC9022517.
32. Naini A, Bartetzko M P, Sanapala S R, Broecker F, Wirtz V, Lisboa M P, Parameswarappa S G, Knopp D, Przygodda J, Hakelberg M, Pan R, Patel A, Chorro L, Illenberger A, Ponce C, Kodali S, Lypowy J, Anderson A S, Donald RGK, von Bonin A, Pereira C L. Semisynthetic Glycoconjugate Vaccine Candidates against Escherichia coli 025B Induce Functional IgG Antibodies in Mice. JACS Au. 2022 Aug. 31; 2 (9): 2135-2151. doi: 10.1021/jacsau.2c00401. PMID: 36186572; PMCID: PMC9516715.
33. Kowarik M, Wetter M, Haeuptle M A, Braun M, Steffen M, Kemmler S, Ravenscroft N, De Benedetto G, Zuppiger M, Sirena D, Cescutti P, Wacker M. The development and characterization of an E. coli O25B bioconjugate vaccine. Glycoconj J. 2021 August; 38 (4): 421-435. doi: 10.1007/s10719-021-09985-9. Epub 2021 Mar. 17. PMID: 33730261; PMCID: PMC8260533.
34. Asadi Karam M R, Habibi M, Bouzari S. Use of flagellin and cholera toxin as adjuvants in intranasal vaccination of mice to enhance protective immune responses against uropathogenic Escherichia coli antigens. Biologicals. 2016 September; 44 (5): 378-86. doi: 10.1016/j.biologicals.2016.06.006. Epub 2016 Jul. 25. PMID: 27461240.
35. Asadi Karam M R, Oloomi M, Mahdavi M, Habibi M, Bouzari S. Vaccination with recombinant FimH fused with flagellin enhances cellular and humoral immunity against urinary tract infection n mice. Vaccine. 2013 Feb. 6; 31 (8): 1210-6. doi: 10.1016/j.vaccine.2012.12.059. Epub 2013 Jan. 7. PMID: 23306365.
36. Karam M R, Oloomi M, Mahdavi M, Habibi M, Bouzari S. Assessment of immune responses of the flagellin (FliC) fused to FimH adhesin of Uropathogenic Escherichia coli. Mol Immunol. 2013 May; 54 (1): 32-9. doi: 10.1016/j.molimm.2012.11.002. Epub 2012 Dec. 3. PMID: 23220068.
37. Eldridge G R, Hughey H, Rosenberger L, Martin S M, Shapiro A M, D'Antonio E, Krejci K G, Shore N, Peterson J, Lukes A S, Starks C M. Safety and immunogenicity of an adjuvanted Escherichia coli adhesin vaccine in healthy women with and without histories of recurrent urinary tract infections: results from a first-in-human phase 1 study. Hum Vaccin Immunother. 2021 May 4; 17 (5): 1262-1270. doi: 10.1080/21645515.2020.1834807. Epub 2020 Dec. 16. PMID: 33325785; PMCID: PMC8078672.
38. Nesta B, Spraggon G, Alteri C, Moriel D G, Rosini R, Veggi D, Smith S, Bertoldi I, Pastorello I, Ferlenghi I, Fontana M R, Frankel G, Mobley H L, Rappuoli R, Pizza M, Serino L, Soriani M. FdeC, a novel broadly conserved Escherichia coli adhesin eliciting protection against urinary tract infections. mBio. 2012 Apr. 10; 3 (2):e00010-12. doi: 10.1128/mBio.00010-12. PMID: 22496310; PMCID: PMC3324786.
39. Roberts J A, Kaack M B, Baskin G, Chapman M R, Hunstad D A, Pinkner J S, Hultgren S J. Antibody responses and protection from pyelonephritis following vaccination with purified Escherichia coli PapDG protein. J Urol. 2004 April; 171 (4): 1682-5. doi: 10.1097/01.ju.0000116123.05160.43. PMID: 15017266; PMCID: PMC2838480.
40. Goluszko P, Goluszko E, Nowicki B, Nowicki S, Popov V, Wang H Q. Vaccination with purified Dr Fimbriae reduces mortality associated with chronic urinary tract infection due to Escherichia coli bearing Dr adhesin. Infect Immun. 2005 January; 73 (1): 627-31. doi: 10.1128/IAI.73.1.627-631.2005. PMID: 15618205; PMCID: PMC538924.
41. Wieser A, Magistro G, Nörenberg D, Hoffmann C, Schubert S. First multi-epitope subunit vaccine against extraintestinal pathogenic Escherichia coli delivered by a bacterial type-3 secretion system (T3S S). Int J Med Microbiol. 2012 January; 302 (1): 10-8. doi: 10.1016/j.ijmm.2011.09.012. Epub 2011 Oct. 13. PMID: 22000741.
42. Mobley H L, Alteri C J. Development of a Vaccine against Escherichia coli Urinary Tract Infections. Pathogens. 2015 Dec. 31; 5 (1): 1. doi: 10.3390/pathogens5010001. PMID: 26729174; PMCID: PMC4810122.
43. Wieser A, Romann E, Magistro G, Hoffmann C, Nörenberg D, Weinert K, Schubert S. A multiepitope subunit vaccine conveys protection against extraintestinal pathogenic Escherichia coli in mice. Infect Immun. 2010 August; 78 (8): 3432-42. doi: 10.1128/IAI.00174-10. Epub 2010 May 24. PMID: 20498257; PMCID: PMC2916271.
44. Habibi M, Asadi Karam M R, Bouzari S. Evaluation of prevalence, immunogenicity and efficacy of FyuA iron receptor in uropathogenic Escherichia coli isolates as a vaccine target against urinary tract infection. Microb Pathog. 2017 September; 110:477-483. doi: 10.1016/j.micpath.2017.07.037. Epub 2017 Jul. 25. PMID: 28754265.
45. Brumbaugh A R, Smith S N, Mobley H L. Immunization with the yersiniabactin receptor, FyuA, protects against pyelonephritis in a murine model of urinary tract infection. Infect Immun. 2013 September; 81 (9): 3309-16. doi: 10.1128/IAI.00470-13. Epub 2013 Jun. 24. PMID: 23798537; PMCID: PMC3754202.
46. Alteri C J, Hagan E C, Sivick K E, Smith S N, Mobley H L. Mucosal immunization with iron receptor antigens protects against urinary tract infection. PLOS Pathog. 2009 September; 5 (9): e1000586. doi: 10.1371/journal.ppat.1000586. Epub 2009 Sep. 18. PMID: 19806177; PMCID: PMC2736566.
47. Mike L A, Smith S N, Sumner C A, Eaton K A, Mobley H L. Siderophore vaccine conjugates protect against uropathogenic Escherichia coli urinary tract infection. Proc Natl Acad Sci USA. 2016 November 22; 113 (47): 13468-13473. doi: 10.1073/pnas. 1606324113. Epub 2016 Nov. 7. PMID: 27821778; PMCID: PMC5127358.
48. Forsyth V S, Himpsl S D, Smith S N, Sarkissian C A, Mike L A, Stocki J A, Sintsova A, Alteri C J, Mobley H L T. Optimization of an Experimental Vaccine To Prevent Escherichia coli Urinary Tract Infection. mBio. 2020 Apr. 28; 11 (2): e00555-20. doi: 10.1128/mBio.00555-20. PMID: 32345645; PMCID: PMC7188996.
49. Mellata M, Mitchell N M, Schödel F, Curtiss R Rd, Pier G B. Novel vaccine antigen combinations elicit protective immune responses against Escherichia coli sepsis. Vaccine. 2016 Jan. 27; 34 (5): 656-662. doi: 10.1016/j.vaccine.2015.12.014. Epub 2015 Dec. 18. PMID: 26707217; PMCID: PMC4715933.
50. O'Hanley P, Lalonde G, Ji G. Alpha-hemolysin contributes to the pathogenicity of piliated digalactoside-binding Escherichia coli in the kidney: efficacy of an alpha-hemolysin vaccine in preventing renal injury in the BALB/c mouse model of pyelonephritis. Infect Immun. 1991 March; 59 (3): 1153-61. doi: 10.1128/iai.59.3.1153-1161.1991. PMID: 1671776; PMCID: PMC258381.
51. Moriel D G, Bertoldi I, Spagnuolo A, Marchi S, Rosini R, Nesta B, Pastorello I, Corea V A, Torricelli G, Cartocci E, Savino S, Scarselli M, Dobrindt U, Hacker J, Tettelin H, Tallon L J, Sullivan S, Wieler L H, Ewers C, Pickard D, Dougan G, Fontana M R, Rappuoli R, Pizza M, Serino L. Identification of protective and broadly conserved vaccine antigens from the genome of extraintestinal pathogenic Escherichia coli. Proc Natl Acad Sci USA. 2010 May 18; 107 (20): 9072-7. doi: 10.1073/pnas.0915077107. Epub 2010 May 3. PMID: 20439758; PMCID: PMC2889118.
52. Smith M A, Weingarten R A, Russo L M, Ventura C L, O'Brien A D. Antibodies against hemolysin and cytotoxic necrotizing factor type 1 (CNF1) reduce bladder inflammation in a mouse model of urinary tract infection with toxigenic uropathogenic Escherichia coli. Infect Immun. 2015 April; 83 (4): 1661-73. doi: 10.1128/IAI.02848-14. Epub 2015 Feb. 9. PMID: 25667267; PMCID: PMC4363410.
53. van den Dobbelsteen GPJM, Fae K C, Serroyen J, van den Nieuwenhof I M, Braun M, Haeuptle M A, Sirena D, Schneider J, Alaimo C, Lipowsky G, Gambillara-Fonck V, Wacker M, Poolman J T. Immunogenicity and safety of a tetravalent E. coli O-antigen bioconjugate vaccine in animal models. Vaccine. 2016 Jul. 29; 34 (35): 4152-4160. doi: 10.1016/j.vaccine.2016.06.067. Epub 2016 Jul. 6. PMID: 27395567.
54. Huttner A, Gambillara V. The development and early clinical testing of the ExPEC4V conjugate vaccine against uropathogenic Escherichia coli. Clin Microbiol Infect. 2018 October; 24 (10): 1046-1050. doi: 10.1016/j.cmi.2018.05.009. Epub 2018 May 24. PMID: 29803843.
55. Inoue M, Ogawa T, Tamura H, Hagiwara Y, Saito Y, Abbanat D, van den Dobbelsteen G, Hermans P, Thoelen S, Poolman J, Ibarra de Palacios P. Safety, tolerability and immunogenicity of the ExPEC4V (JNJ-63871860) vaccine for prevention of invasive extraintestinal pathogenic Escherichia coli disease: A phase 1, randomized, double-blind, placebo-controlled study in healthy Japanese participants. Hum Vaccin Immunother. 2018; 14 (9): 2150-2157. doi: 10.1080/21645515.2018.1474316. Epub 2018 Jun. 28. PMID: 29771596; PMCID: PMC6183137.
56. Huttner A, Hatz C, van den Dobbelsteen G, Abbanat D, Hornacek A, Frölich R, Dreyer A M, Martin P, Davies T, Fae K, van den Nieuwenhof I, Thoelen S, de Vallière S, Kuhn A, Bernasconi E, Viereck V, Kavvadias T, Kling K, Ryu G, Hülder T, Gröger S, Scheiner D, Alaimo C, Harbarth S, Poolman J, Fonck V G. Safety, immunogenicity, and preliminary clinical efficacy of a vaccine against extraintestinal pathogenic Escherichia coli in women with a history of recurrent urinary tract infection: a randomised, single-blind, placebo-controlled phase 1b trial. Lancet Infect Dis. 2017 May; 17 (5): 528-537. doi: 10.1016/S1473-3099 (17) 30108-1. Epub 2017 Feb. 24. PMID: 28238601.
57. Frenck R W Jr, Ervin J, Chu L, Abbanat D, Spiessens B, Go O, Haazen W, van den Dobbelsteen G, Poolman J, Thoelen S, Ibarra de Palacios P. Safety and immunogenicity of a vaccine for extra-intestinal pathogenic Escherichia coli (ESTELLA): a phase 2 randomised controlled trial. Lancet Infect Dis. 2019 June; 19 (6): 631-640. doi: 10.1016/S1473-3099 (18) 30803-X. Epub 2019 May 9. PMID: 31079947.
58. B Smith W, Abbanat D, Spiessens B, Go O, Haazen W, de Rosa T, Fae K, Poolman J, Thoelen S, de Palacios P I. 2712. Safety and Immunogenicity of two Doses of ExPEC4V Vaccine Against Extraintestinal Pathogenic Escherichia coli Disease in Healthy Adult Participants. Open Forum Infect Dis. 2019 Oct. 23; 6 (Suppl 2): S954. doi: 10.1093/ofid/ofz360.2389. PMCID: PMC6810236.
59. Ristow L C, Welch R A. Hemolysin of uropathogenic Escherichia coli: A cloak or a dagger? Biochim Biophys Acta. 2016 March; 1858 (3): 538-45. doi: 10.1016/j.bbamem.2015.08.015. Epub 2015 Aug. 20. PMID: 26299820.
60. Wang C, Li Q, Lv J, Sun X, Cao Y, Yu K, Miao C, Zhang Z S, Yao Z, Wang Q. Alpha-hemolysin of uropathogenic Escherichia coli induces G M-CSF-mediated acute kidney injury. Mucosal Immunol. 2020 January; 13 (1): 22-33. doi: 10.1038/s41385-019-0225-6. Epub 2019 Nov. 12. PMID: 31719643; PMCID: PMC6914670.
61. Wiles T J, Dhakal B K, Eto D S, Mulvey M A. Inactivation of host Akt/protein kinase B signaling by bacterial pore-forming toxins. Mol Biol Cell. 2008 April; 19 (4): 1427-38. doi: 10.1091/mbc.e07-07-0638. Epub 2008 Jan. 30. PMID: 18234841; PMCID: PMC2291440.
62. Dhakal B K, Mulvey M A. The UPEC pore-forming toxin α-hemolysin triggers proteolysis of host proteins to disrupt cell adhesion, inflammatory, and survival pathways. Cell Host Microbe. 2012 Jan. 19; 11 (1): 58-69. doi: 10.1016/j.chom.2011.12.003. PMID: 22264513; PMCID: PMC3266558.
63. Nagamatsu K, Hannan T J, Guest R L, Kostakioti M, Hadjifrangiskou M, Binkley J, Dodson K, Raivio T L, Hultgren S J. Dysregulation of Escherichia coli α-hemolysin expression alters the course of acute and persistent urinary tract infection. Proc Natl Acad Sci USA. 2015 Feb. 24; 112 (8): E871-80. doi: 10.1073/pnas.1500374112. Epub 2015 Feb. 9. PMID: 25675528; PMCID: PMC4345586.
64. Johnsen N, Hamilton A D M, Greve A S, Christensen M G, Therkildsen J R, Wehmöller J, Skals M, Praetorius H A. α-Haemolysin production, as a single factor, causes fulminant sepsis in a model of Escherichia coli-induced bacteraemia. Cell Microbiol. 2019 June; 21 (6): e13017. doi: 10.1111/cmi.13017. Epub 2019 Mar. 4. PMID: 30761726.
65. Smith Y C, Rasmussen S B, Grande K K, Conran R M, O'Brien A D. Hemolysin of uropathogenic Escherichia coli evokes extensive shedding of the uroepithelium and hemorrhage in bladder tissue within the first 24 hours after intraurethral inoculation of mice. Infect Immun. 2008 July; 76 (7): 2978-90. doi: 10.1128/IAI.00075-08. Epub 2008 Apr. 28. PMID: 18443089; PMCID: PMC2446707.
66. Bien J, Sokolova O, Bozko P. Role of Uropathogenic Escherichia coli Virulence Factors in Development of Urinary Tract Infection and Kidney Damage. Int J Nephrol. 2012; 2012:681473. doi: 10.1155/2012/681473. Epub 2012 Mar. 8. PMID: 22506110; PMCID: PMC3312279.
67. Daga A P, Koga V L, Soncini J G M, de Matos C M, Perugini M R E, Pelisson M, Kobayashi R K T, Vespero E C. Escherichia coli Bloodstream Infections in Patients at a University Hospital: Virulence Factors and Clinical Characteristics. Front Cell Infect Microbiol. 2019 Jun. 6; 9:191. doi: 10.3389/fcimb.2019.00191. PMID: 31245301; PMCID: PMC6563721.
68. Li B, Lu Y, Lan F, He Q, Li C, Cao Y. Prevalence and characteristics of ST131 clone among unselected clinical Escherichia coli in a Chinese university hospital. Antimicrob Resist Infect Control. 2017 Nov. 15; 6:118. doi: 10.1186/s13756-017-0274-0. PMID: 29167743; PMCID: PMC5688664.
69. Rijavec M, Müller-Premru M, Zakotnik B, Žgur-Bertok D. Virulence factors and biofilm production among Escherichia coli strains causing bacteraemia of urinary tract origin. J Med Microbiol. 2008 November; 57 (Pt 11): 1329-1334. doi: 10.1099/jmm.0.2008/002543-0. PMID: 18927408.
70. Karam M R A, Habibi M, Bouzari S. Relationships between Virulence Factors and Antimicrobial Resistance among Escherichia coli Isolated from Urinary Tract Infections and Commensal Isolates in Tehran, Iran. Osong Public Health Res Perspect. 2018 October; 9 (5): 217-224. doi: 10.24171/j.phrp.2018.9.5.02. PMID: 30402376; PMCID: PMC6202021.
71. Kim B, Kim J H, Lee Y. Virulence Factors Associated With Escherichia coli Bacteremia and Urinary Tract Infection. Ann Lab Med. 2022 Mar. 1; 42 (2): 203-212. doi: 10.3343/alm.2022.42.2.203. PMID: 34635614; PMCID: PMC8548248.
72. Moeinizadeh H, Shaheli M. Frequency of hlyA, hlyB, hlyC and hlyD genes in uropathogenic Escherichia coli isolated from UTI patients in Shiraz. GMS Hyg Infect Control. 2021 Aug. 30; 16: Doc25. doi: 10.3205/dgkh000396. PMID: 34549018; PMCID: PMC8430235.
73. Lavigne J P, Boutet-Dubois A, Laouini D, Combescure C, Bouziges N, Marès P, Sotto A. Virulence potential of Escherichia coli strains causing asymptomatic bacteriuria during pregnancy. J Clin Microbiol. 2011 November; 49 (11): 3950-3. doi: 10.1128/JCM.00892-11. Epub 2011 Sep. 14. PMID: 21918033; PMCID: PMC3209114.
74. Nhu N T K, Phan M D, Forde B M, Murthy A M V, Peters K M, Day C J, Poole J, Kidd T J, Welch R A, Jennings M P, Ulett G C, Sweet M J, Beatson S A, Schembri M A. Complex Multilevel Control of Hemolysin Production by Uropathogenic Escherichia coli. mBio. 2019 Oct. 1; 10 (5): e02248-19. doi: 10.1128/mBio.02248-19. PMID: 31575773; PMCID: PMC6775461.
75. Clark J R, Maresso A M. Comparative Pathogenomics of Escherichia coli: Polyvalent Vaccine Target Identification through Virulome Analysis. Infect Immun. 2021 Jul. 15; 89 (8): e0011521. doi: 10.1128/IAI.00115-21. Epub 2021 Jul. 15. PMID: 33941580; PMCID: PMC8281228.
76. Thomas S, Bakkes P J, Smits S H, Schmitt L. Equilibrium folding of pro-HlyA from Escherichia coli reveals a stable calcium ion dependent folding intermediate. Biochim Biophys Acta. 2014 September; 1844 (9): 1500-10. doi: 10.1016/j.bbapap.2014.05.006. Epub 2014 May 24. PMID: 24865936.
77. Xing Y, Clark J R, Chang J D, Chirman D M, Green S, Zulk J J, Jelinski J, Patras K A, Maresso A W. Broad protective vaccination against systemic Escherichia coli with autotransporter antigens. PLOS Pathog. 2023 Feb. 17; 19 (2): e1011082. doi: 10.1371/journal.ppat. 1011082. PMID: 36800400; PMCID: PMC9937491.
78. Johnson J R, Johnston B, Clabots C, Kuskowski M A, Castanheira M. Escherichia coli sequence type ST131 as the major cause of serious multidrug-resistant E. coli infections in the United States. Clin Infect Dis. 2010 Aug. 1; 51 (3): 286-94. pmid: 20572763
79. Mulvey M A, Schilling J D, Hultgren S J. Establishment of a persistent Escherichia coli reservoir during the acute phase of a bladder infection. Infect Immun. 2001 July; 69 (7): 4572-9. doi: 10.1128/IAI.69.7.4572-4579.2001. PMID: 11402001; PMCID: PMC98534.
80. Mobley H L, Green D M, Trifillis A L, Johnson D E, Chippendale G R, Lockatell C V, Jones B D, Warren J W. Pyelonephritogenic Escherichia coli and killing of cultured human renal proximal tubular epithelial cells: role of hemolysin in some strains. Infect Immun. 1990 May; 58 (5): 1281-9. pmid: 2182540
81. O'Leary N A, Wright M W, Brister J R, Ciufo S, Haddad D, McVeigh R. Reference sequence (RefSeq) database at NCBI: current status, taxonomic expansion, and functional annotation. Nucleic Acids Res. 2016 Jan. 4; 44 (D1): D733-45. doi: 10.1093/nar/gkv1189. Epub 2015 Nov. 8. PMID: 26553804; PMCID: PMC4702849.
82. Jolley K A, Maiden M C. BIGSdb: Scalable analysis of bacterial genome variation at the population level. BMC Bioinformatics. 2010 Dec. 10; 11:595. doi: 10.1186/1471-2105-11-595. PMID: 21143983; PMCID: PMC3004885.
83. Altschul S F, Gish W, Miller W, Myers E W, Lipman D J. Basic local alignment search tool. J Mol Biol. 1990 Oct. 5; 215 (3): 403-10. doi: 10.1016/S0022-2836 (O5) 80360-2. PMID: 2231712.
84. Altschul S F, Madden T L, Schäffer A A, Zhang J, Zhang Z, Miller W, Lipman D J. Gapped BLAST and PSI-BLAST: a new generation of protein database search programs. Nucleic Acids Res. 1997 Sep. 1; 25 (17): 3389-402. doi: 10.1093/nar/25.17.3389. PMID: 9254694; PMCID: PMC146917.
85. Camacho C, Coulouris G, Avagyan V, Ma N, Papadopoulos J, Bealer K, Madden T L. BLAST+: architecture and applications. BMC Bioinformatics. 2009 Dec. 15; 10:421. doi: 10.1186/1471-2105-10-421. PMID: 20003500; PMCID: PMC2803857.
86. Alanjary M, Steinke K, Ziemert N. AutoMLST: an automated web server for generating multi-locus species trees highlighting natural product potential. Nucleic Acids Res. 2019 Jul. 2; 47 (W1): W276-W282. doi: 10.1093/nar/gkz282. PMID: 30997504; PMCID: PMC6602446.
87. Jumper J, Evans R, Pritzel A, Green T, Figurnov M, Ronneberger O. Highly accurate protein structure prediction with AlphaFold. Nature. 2021 August; 596 (7873): 583-589. doi: 10.1038/s41586-021-03819-2. Epub 2021 Jul. 15. PMID: 34265844; PMCID: PMC8371605.
88. Mirdita M, Schütze K, Moriwaki Y, Heo L, Ovchinnikov S, Steinegger M. ColabFold: making protein folding accessible to all. Nat Methods. 2022 June; 19 (6): 679-682. doi: 10.1038/s41592-022-01488-1. Epub 2022 May 30. PMID: 35637307; PMCID: PMC9184281.
89. Steinegger M, Söding J. MMseqs2 enables sensitive protein sequence searching for the analysis of massive data sets. Nat Biotechnol. 2017 November; 35 (11): 1026-1028. doi: 10.1038/nbt.3988. Epub 2017 Oct. 16. PMID: 29035372.
90. Steinegger M, Söding J. Clustering huge protein sequence sets in linear time. Nat Commun. 2018 Jun. 29; 9 (1): 2542. doi: 10.1038/s41467-018-04964-5. PMID: 29959318; PMCID: PMC6026198.
91. Mariani V, Biasini M, Barbato A, Schwede T. IDDT: a local superposition-free score for comparing protein structures and models using distance difference tests. Bioinformatics. 2013 Nov. 1; 29 (21): 2722-8. doi: 10.1093/bioinformatics/btt473. Epub 2013 Aug. 27. PMID: 23986568; PMCID: PMC3799472.
92. Hornak V, Abel R, Okur A, Strockbine B, Roitberg A, Simmerling C. Comparison of multiple Amber force fields and development of improved protein backbone parameters. Proteins. 2006 Nov. 15; 65 (3): 712-25. doi: 10.1002/prot.21123. PMID: 16981200; PMCID: PMC4805110.
93. Berman H M, Westbrook J, Feng Z, Gilliland G, Bhat T N, Weissig H, Shindyalov I N, Bourne P E. The Protein Data Bank. Nucleic Acids Res. 2000 Jan. 1; 28 (1): 235-42. doi: 10.1093/nar/28.1.235. PMID: 10592235; PMCID: PMC102472.
94. Zhang Y, Skolnick J. T M-align: a protein structure alignment algorithm based on the T M-score. Nucleic Acids Res. 2005 Apr. 22; 33 (7): 2302-9. doi: 10.1093/nar/gki524. PMID: 15849316; PMCID: PMC1084323.
95. van Kempen M, Kim S S, Tumescheit C, Mirdita M, Lee J, Gilchrist C L M, Söding J, Steinegger M. Fast and accurate protein structure search with Foldseek. Nat Biotechnol. 2023 May 8. doi: 10.1038/s41587-023-01773-0. Epub ahead of print. PMID: 37156916.
96. UniProt Consortium. UniProt: the Universal Protein Knowledgebase in 2023. Nucleic Acids Res. 2023 Jan. 6; 51 (D1): D523-D531. doi: 10.1093/nar/gkac1052. PMID: 36408920; PMCID: PMC9825514.
97. Meng E C, Pettersen E F, Couch G S, Huang C C, Ferrin T E. Tools for integrated sequence-structure analysis with UCSF Chimera. BMC Bioinformatics. 2006 Jul. 12; 7:339. doi: 10.1186/1471-2105-7-339. PMID: 16836757; PMCID: PMC1570152.
98. Kon E, Levy Y, Elia U, Cohen H, Hazan-Halevy I, Aftalion M, Ezra A, Bar-Haim E, Naidu G S, Diesendruck Y, Rotem S, Ad-El N, Goldsmith M, Mamroud E, Peer D, Cohen O. A single-dose F1-based mRNA-LNP vaccine provides protection against the lethal plague bacterium. Sci Adv. 2023 Mar. 10; 9 (10): eadg1036. doi: 10.1126/sciadv.adg1036. Epub 2023 Mar. 8. PMID: 36888708; PMCID: PMC9995031.
99. Emsley P, Charles I G, Fairweather N F, Isaacs N W. Structure of Bordetella pertussis virulence factor P.69 pertactin. Nature. 1996 May 2; 381 (6577): 90-2. doi: 10.1038/381090a0. PMID: 8609998.
100. Esposito S, Stefanelli P, Fry N K, Fedele G, He Q, Paterson P, Tan T, Knuf M, Rodrigo C, Weil Olivier C, Flanagan K L, Hung I, Lutsar I, Edwards K, O'Ryan M, Principi N; World Association of Infectious Diseases and Immunological Disorders (WAidid) and the Vaccine Study Group of the European Society of Clinical Microbiology and Infectious Diseases (EVASG). Pertussis Prevention: Reasons for Resurgence, and Differences in the Current Acellular Pertussis Vaccines. Front Immunol. 2019 Jul. 3; 10:1344. doi: 10.3389/fimmu.2019.01344. PMID: 31333640; PMCID: PMC6616129.
101. Goldsmith J A, DiVenere A M, Maynard J A, Mclellan J S. Structural basis for antibody binding to adenylate cyclase toxin reveals RTX linkers as neutralization-sensitive epitopes. PLOS Pathog. 2021 Sep. 21; 17 (9): e1009920. doi: 10.1371/journal.ppat. 1009920. PMID: 34547035; PMCID: PMC8486122.
102. Novak W R, Bhattacharyya B, Grilley D P, Weaver T M. Proteolysis of truncated hemolysin A yields a stable dimerization interface. Acta Crystallogr F Struct Biol Commun. 2017 Mar. 1; 73 (Pt 3): 138-145. doi: 10.1107/S2053230X17002102. Epub 2017 February 21. PMID: 28291749; PMCID: PMC5349307.
103. Pourhassan N Z, Hachani E, Spitz O, Smits S H J, Schmitt L. Investigations on the substrate binding sites of hemolysin B, an ABC transporter, of a type 1 secretion system. Front Microbiol. 2022 Dec. 1; 13:1055032. doi: 10.3389/fmich.2022.1055032. PMID: 36532430; PMCID: PMC9751043.
104 Gentschev I, Dietrich G, Goebel W. The E. coli alpha-hemolysin secretion system and its use in vaccine development. Trends Microbiol. 2002 January; 10 (1): 39-45. doi: 10.1016/s0966-842x(01) 02259-4. PMID: 11755084
105. Pourhassan N Z, Smits S H J, Ahn J H, Schmitt L. Biotechnological applications of type 1 secretion systems. Biotechnol Adv. 2021 December; 53:107864. doi: 10.1016/j.biotechadv.2021.107864. Epub 2021 Nov. 10. PMID: 34767962.
106. Zhao H, Lee J, Chen J. The hemolysin A secretion system is a multi-engine pump containing three ABC transporters. Cell. 2022 Sep. 1; 185 (18): 3329-3340.e13. doi: 10.1016/j.cell.2022.07.017. PMID: 36055198.
107. Pourhassan N Z, Hachani E, Spitz O, Smits S H J, Schmitt L. Investigations on the substrate binding sites of hemolysin B, an ABC transporter, of a type 1 secretion system. Front Microbiol. 2022 Dec. 1; 13:1055032. doi: 10.3389/fmich.2022.1055032. PMID: 36532430; PMCID: PMC9751043.
108. Spitz O, Erenburg I N, Kanonenberg K, Peherstorfer S, Lenders M H H, Reiners J, Ma M, Luisi B F, Smits S H J, Schmitt L. Identity Determinants of the Translocation Signal for a Type 1 Secretion System. Front Physiol. 2022 Feb. 10; 12:804646. doi: 10.3389/fphys.2021.804646. PMID: 35222063; PMCID: PMC8870123.
109. Thomas S, Holland I B, Schmitt L. The Type 1 secretion pathway—the hemolysin system and beyond. Biochim Biophys Acta. 2014 August; 1843 (8): 1629-41. doi: 10.1016/j.bbamcr.2013.09.017. Epub 2013 Oct. 12. PMID: 24129268.
110. Doumith M, Day M, Ciesielczuk H, Hope R, Underwood A, Reynolds R, Wain J, Livermore D M, Woodford N. Rapid identification of major Escherichia coli sequence types causing urinary tract and bloodstream infections. J Clin Microbiol. 2015 January; 53 (1): 160-6. doi: 10.1128/JCM.02562-14. Epub 2014 Oct. 29. PMID: 25355761; PMCID: PMC4290915.
111. García V, Grønnemose R B, Torres-Puig S, Kudirkiene E, Piantelli M, Ahmed S, Andersen T E, Møller-Jensen J, Olsen J E, Herrero-Fresno A. Genome-wide analysis of fitness-factors in uropathogenic Escherichia coli during growth in laboratory media and during urinary tract infections. Microb Genom. 2021 December; 7 (12): 000719. doi: 10.1099/mgen.0.000719. PMID: 34928200; PMCID: PMC8767336.
112. Riley L W. Pandemic lineages of extraintestinal pathogenic Escherichia coli. Clin Microbiol Infect. 2014 May; 20 (5): 380-90. doi: 10.1111/1469-0691.12646. PMID: 24766445.
113. Mobley H L, Green D M, Trifillis A L, Johnson D E, Chippendale G R, Lockatell C V, Jones B D, Warren J W. Pyelonephritogenic Escherichia coli and killing of cultured human renal proximal tubular epithelial cells: role of hemolysin in some strains. Infect Immun. 1990 May; 58 (5): 1281-9. doi: 10.1128/iai.58.5.1281-1289.1990. PMID: 2182540; PMCID: PMC258621.
114. Welch R A, Burland V, Plunkett G 3rd, Redford P, Roesch P, Rasko D, Buckles E L, Liou S R, Boutin A, Hackett J, Stroud D, Mayhew G F, Rose D J, Zhou S, Schwartz D C, Perna N T, Mobley H L, Donnenberg M S, Blattner F R. Extensive mosaic structure revealed by the complete genome sequence of uropathogenic Escherichia coli. Proc Natl Acad Sci USA. 2002 Dec. 24; 99 (26):17020-4. doi: 10.1073/pnas.252529799. Epub 2002 Dec. 5. PMID: 12471157; PMCID: PMC139262.
115. Croxall G, Hale J, Weston V, Manning G, Cheetham P, Achtman M, McNally A. Molecular epidemiology of extraintestinal pathogenic Escherichia coli isolates from a regional cohort of elderly patients highlights the prevalence of ST131 strains with increased antimicrobial resistance in both community and hospital care settings. J Antimicrob Chemother. 2011 November; 66 (11): 2501-8. doi: 10.1093/jac/dkr349. Epub 2011 Aug. 22. PMID: 21862477.
116. Gibreel T M, Dodgson A R, Cheesbrough J, Fox A J, Bolton F J, Upton M. Population structure, virulence potential and antibiotic susceptibility of uropathogenic Escherichia coli from Northwest England. J Antimicrob Chemother. 2012 February; 67 (2): 346-56. doi: 10.1093/jac/dkr451. Epub 2011 Oct. 25. PMID: 22028202.
117. Klein R D, Hultgren S J. Urinary tract infections: microbial pathogenesis, host-pathogen interactions and new treatment strategies. Nat Rev Microbiol. 2020 April; 18 (4): 211-226. doi: 10.1038/s41579-020-0324-0. Epub 2020 Feb. 18. PMID: 32071440; PMCID: PMC7942789.
118. Russell S K, Harrison J K, Olson B S, Lee H J, O'Brien V P, Xing X, Livny J, Yu L, Roberson E D O, Bomjan R, Fan C, Sha M, Estfanous S, Amer A O, Colonna M, Stappenbeck T S, Wang T, Hannan T J, Hultgren S J. Uropathogenic Escherichia coli infection-induced epithelial trained immunity impacts urinary tract disease outcome. Nat Microbiol. 2023 Apr. 10. doi: 10.1038/s41564-023-01346-6. Epub ahead of print. PMID: 37037942.
119 Llor C, Bjerrum L. Antimicrobial resistance: risk associated with antibiotic overuse and initiatives to reduce the problem. Ther Adv Drug Saf. 2014 December; 5 (6): 229-41. doi: 10.1177/2042098614554919. PMID: 25436105; PMCID: PMC4232501.
120. Doua J, Geurtsen J, Rodriguez-Baño J, Cornely O A, Go O, Gomila-Grange A, Kirby A, Hermans P, Gori A, Zuccaro V, Gravenstein S, Bonten M, Poolman J, Sarnecki M. Epidemiology, Clinical Features, and Antimicrobial Resistance of Invasive Escherichia Coli Disease in Patients Admitted in Tertiary Care Hospitals. Open Forum Infect Dis. 2023 Jan. 27; 10 (2): ofad026. doi: 10.1093/ofid/ofad026. PMID: 36817744; PMCID: PMC9933942.
121. Wang S, Zhao S, Zhou Y, Jin S, Ye T, Pan X. Antibiotic resistance spectrum of E. coli strains from different samples and age-grouped patients: a 10-year retrospective study. BMJ Open. 2023 Apr. 12; 13 (4): e067490.
122. García V, Stærk K, Alobaidallah M S A, Grønnemose R B, Guerra P R, Andersen T E, Olsen J E, Herrero-Fresno A. Genome-wide analysis of fitness factors in uropathogenic Escherichia coli in a pig urinary tract infection model. Microbiol Res. 2022 December; 265:127202. doi: 10.1016/j.micres.2022.127202. Epub 2022 Sep. 15. PMID: 36167007.
123. Shea A E, Marzoa J, Himpsl S D, Smith S N, Zhao L, Tran L, Mobley H L T. Escherichia coli CFT073 Fitness Factors during Urinary Tract Infection: Identification Using an Ordered Transposon Library. Appl Environ Microbiol. 2020 Jun. 17; 86 (13): e00691-20. doi: 10.1128/AEM.00691-20. PMID: 32358013; PMCID: PMC7301846.
124. O'Brien V P, Dorsey D A, Hannan T J, Hultgren S J. Host restriction of Escherichia coli recurrent urinary tract infection occurs in a bacterial strain-specific manner. PLOS Pathog. 2018 Dec. 13; 14 (12): e1007457. doi: 10.1371/journal.ppat. 1007457. PMID: 30543708; PMCID: PMC6292575.
125. Upadhyay I, Lauder K L, Li S, Ptacek G, Zhang W. Intramuscularly Administered Enterotoxigenic Escherichia coli (ETEC) Vaccine Candidate MecVax Prevented H10407 Intestinal Colonization in an Adult Rabbit Colonization Model. Microbiol Spectr. 2022 Aug. 31; 10 (4): e0147322. doi: 10.1128/spectrum.01473-22. Epub 2022 Jun. 28. PMID: 35762781; PMCID: PMC9431210.
126. Lee T, Gutiérrez R L, Maciel M, Poole S, Testa K J, Trop S, Duplessis C, Lane A, Riddle M S, Hamer M, Alcala A, Prouty M, Maier N, Erdem R, Louis Bourgeois A, Porter C K. Safety and immunogenicity of intramuscularly administered CS6 subunit vaccine with a modified heat-labile enterotoxin from enterotoxigenic Escherichia coli. Vaccine. 2021 Sep. 15; 39 (39): 5548-5556. doi: 10.1016/j.vaccine.2021.08.032. Epub 2021 Aug. 18. PMID: 34419306; PMCID: PMC8461560.
127. Nizard M, Diniz M O, Roussel H, Tran T, Ferreira L C, Badoual C, Tartour E. Mucosal vaccines: novel strategies and applications for the control of pathogens and tumors at mucosal sites. Hum Vaccin Immunother. 2014; 10 (8): 2175-87. doi: 10.4161/hv.29269. PMID: 25424921; PMCID: PMC4896761.
128. Lycke N. Recent progress in mucosal vaccine development: potential and limitations. Nat Rev Immunol. 2012 Jul. 25; 12 (8): 592-605. doi: 10.1038/nri3251. PMID: 22828912.
129. Verma S K, Mahajan P, Singh N K, Gupta A, Aggarwal R, Rappuoli R, Johri A K. New-age vaccine adjuvants, their development, and future perspective. Front Immunol. 2023 Feb. 24; 14:1043109. doi: 10.3389/fimmu.2023.1043109. PMID: 36911719; PMCID: PMC9998920.
130. Lindblad E B. Aluminium adjuvants—in retrospect and prospect. Vaccine. 2004 Sep. 9; 22 (27-28): 3658-68. doi: 10.1016/j.vaccine.2004.03.032. PMID: 15315845.
131. Forsyth V S, Himpsl S D, Smith S N, Sarkissian C A, Mike L A, Stocki J A, Sintsova A, Alteri C J, Mobley H L T. Optimization of an Experimental Vaccine To Prevent Escherichia coli Urinary Tract Infection. mBio. 2020 Apr. 28; 11 (2): e00555-20. doi: 10.1128/mBio.00555-20. PMID: 32345645; PMCID: PMC7188996.
132. Wu J, Hayes B W, Phoenix C, Macias G S, Miao Y, Choi H W, Hughes F M Jr, Todd Purves J, Lee Reinhardt R, Abraham S N. A highly polarized TH2 bladder response to infection promotes epithelial repair at the expense of preventing new infections. Nat Immunol. 2020 June; 21 (6): 671-683. doi: 10.1038/s41590-020-0688-3. Epub 2020 May 18. PMID: 32424366; PMCID: PMC7480508.
133. Komai-Koma M, Ji Y, Cao H, Liu Z, McSharry C, Xu D. Monophosphoryl lipid A directly regulates Th1 cytokine production in human CD4+ T-cells through Toll-like receptor 2 and 4. Immunobiology. 2021 September; 226 (5): 152132. doi: 10.1016/j.imbio.2021.152132. Epub 2021 Aug. 30. PMID: 34478947.
134. Xing Y, Clark J R, Chang J D, Chirman D M, Green S, Zulk J J, Jelinski J, Patras K A, Maresso A W. Broad protective vaccination against systemic Escherichia coli with autotransporter antigens. PLOS Pathog. 2023 Feb. 17; 19 (2): e1011082. doi: 10.1371/journal.ppat.1011082. PMID: 36800400; PMCID: PMC9937491, incorporated herein by reference.
135. Progress toward the development of an effective vaccine for Extraintestinal pathogenic E. coli (ExPEC): The application of the multiple-protein subunits vaccine in different murine models. Yikun Xing, Justin R. Clark, James D. Chang, Jacob J. Zulk, Dylan M. Chirman, Felipe-Andres Piedra, Kathryn A. Patras, Anthony W. Maresso bioRxiv 2023.05.31.543151; doi, incorporated herein by reference.

What is claimed is:

1. A method of treating, preventing, reducing the risk of, delaying the onset of, and/or reducing the severity of an infection in an individual of a bacteria from the Gammaproteobacteria Class, comprising the step of administering to the individual an effective amount of a composition comprising a non-acylated/inactive form alpha-hemolysin (HlyA) and/or a non-acylated/inactive form or functional fragment thereof.

2. The method of claim 1, wherein the inactive HlyA is from the absence of the catalytic acylation enzyme, HlyC.

3. The method of claim 1 or 2, wherein the inactive HlyA is non-acylated.

4. The method of any one of claims 1-3, wherein the inactive HlyA is not capable of inducing pore formation.

5. The method of any one of claims 1-3, wherein the inactive HlyA is not capable of inducing calcium influx.

6. The method of any one of claims 1-5, wherein the bacteria is in the Enterobacterales Order.

7. The method of any one of claims 1-6, wherein the bacteria is in the Enterobacteriaceae family.

8. The method of any one of claims 1-7, wherein the bacteria is in the Genus Escherichia, Vibrio Shigella, Salmonella, Yersinia, Enterobacter, Morganella, or Citerobacter.

9. The method of any one of claims 1-8, wherein the bacteria is Escherichia coli, Vibrio parahaemolyticus, Shigella sonnei, Shigella flexneri, Shigella boydii, Shigella dysenteriae, Salmonella bongori, Salmonella enterica, Yersinia pestis, Yersinia enterocolitica, Yersinia pseudotuberculosis, Enterobacter huaxiensis, Enterobacter chuandaensis, Enterobacter aerogenes, Enterobacter amnigenus, Enterobacter arachidis, Enterobacter asburiae, Enterobacter carcinogenus, Enterobacter cloacae, Enterobacter cowanii, Enterobacter dissolvans, Enterobacter gergoviae, Enterobacter helveticus, Enterobacter hormaechei, Enterobacter kobei, Enterobacter ludwigii, Enterobacter mori, Enterobacter nimipressuralis, Enterobacter oryzae, Enterobacter pulveris, Enterobacter pyrinus, Enterobacter radicincitans, Enterobacter soli, Enterobacter taylorae, Enterobacter turicensis, Morganella morganii, Citerobacter freundii, Citerobacter koseri, Citerobacter amalonaticus, Citerobacter farmeri, Citerobacter youngae, Citerobacter braakii, Citerobacter werkmanii, Citerobacter sedlakii, Citerobacter rodentium, Citerobacter gillenii, or Citerobacter murliniae.

10. The method of any one of claims 1-9, wherein the HlyA is from a bacteria member of the Enterobacterales Order.

11. The method of any one of claims 1-10, wherein the HlyA is from a bacteria member of the Enterobacteriaceae family.

12. The method of any one of claims 1-11, wherein the HlyA is from a bacteria member of the Genus Escherichia, Vibrio Shigella, Salmonella, Yersinia, Enterobacter, Morganella, or Citerobacter.

13. The method of any one of claims 1-12, wherein the HlyA is from Escherichia coli, Vibrio parahaemolyticus, Shigella sonnei, Shigella flexneri, Shigella boydii, Shigella dysenteriae, Salmonella bongori, Salmonella enterica, Yersinia pestis, Yersinia enterocolitica, Yersinia pseudotuberculosis, Enterobacter huaxiensis, Enterobacter chuandaensis, Enterobacter aerogenes, Enterobacter amnigenus, Enterobacter arachidis, Enterobacter asburiae, Enterobacter carcinogenus, Enterobacter cloacae, Enterobacter cowanii, Enterobacter dissolvans, Enterobacter gergoviae, Enterobacter helveticus, Enterobacter hormaechei, Enterobacter kobei, Enterobacter ludwigii, Enterobacter mori, Enterobacter nimipressuralis, Enterobacter oryzae, Enterobacter pulveris, Enterobacter pyrinus, Enterobacter radicincitans, Enterobacter soli, Enterobacter taylorae, Enterobacter turicensis, Morganella morganii, Citerobacter freundii, Citerobacter koseri, Citerobacter amalonaticus, Citerobacter farmeri, Citerobacter youngae, Citerobacter braakii, Citerobacter werkmanii, Citerobacter sedlakii, Citerobacter rodentium, Citerobacter gillenii, or Citerobacter murliniae.

14. A method of vaccinating an individual for a pathogenic infection of a bacteria from the Gammaproteobacteria Class, comprising the step of administering to the individual an effective amount of a composition comprising a non-acylated/inactive form of alpha-hemolysin (HlyA) and/or a non-acylated/inactive form or functional fragment thereof.

15. The method of claim 14, wherein the bacteria is in the Enterobacterales Order.

16. The method of claim 14 or 15, wherein the bacteria is in the Enterobacteriaceae family.

17. The method of any one of claims 14-16, wherein the bacteria is in the Genus Escherichia, Vibrio Shigella, Salmonella, Yersinia, Enterobacter, Morganella, or Citerobacter.

18. The method of any one of claims 14-17, wherein the bacteria is Escherichia coli, Vibrio parahaemolyticus, Shigella sonnei, Shigella flexneri, Shigella boydii, Shigella dysenteriae, Salmonella bongori, Salmonella enterica, Yersinia pestis, Yersinia enterocolitica, Yersinia pseudotuberculosis, Enterobacter huaxiensis, Enterobacter chuandaensis, Enterobacter aerogenes, Enterobacter amnigenus, Enterobacter arachidis, Enterobacter asburiae, Enterobacter carcinogenus, Enterobacter cloacae, Enterobacter cowanii, Enterobacter dissolvans, Enterobacter gergoviae, Enterobacter helveticus, Enterobacter hormaechei, Enterobacter kobei, Enterobacter ludwigii, Enterobacter mori, Enterobacter nimipressuralis, Enterobacter oryzae, Enterobacter pulveris, Enterobacter pyrinus, Enterobacter radicincitans, Enterobacter soli, Enterobacter taylorae, Enterobacter turicensis, Morganella morganii, Citerobacter freundii, Citerobacter koseri, Citerobacter amalonaticus, Citerobacter farmeri, Citerobacter youngae, Citerobacter braakii, Citerobacter werkmanii, Citerobacter sedlakii, Citerobacter rodentium, Citerobacter gillenii, or Citerobacter murliniae.

19. The method of any one of claims 14-18, wherein the HlyA is from a bacteria member of the Enterobacterales Order.

20. The method of any one of claims 14-19, wherein the HlyA is from a bacteria member of the Enterobacteriaceae family.

21. The method of any one of claims 14-20, wherein the HlyA is from a bacteria member of the Genus Escherichia, Vibrio Shigella, Salmonella, Yersinia, Enterobacter, Morganella, or Citerobacter.

22. The method of any one of claims 14-21, wherein the Hly A is from Escherichia coli, Vibrio parahaemolyticus, Shigella sonnei, Shigella flexneri, Shigella boydii, Shigella dysenteriae, Salmonella bongori, Salmonella enterica, Yersinia pestis, Yersinia enterocolitica, Yersinia pseudotuberculosis, Enterobacter huaxiensis, Enterobacter chuandaensis, Enterobacter aerogenes, Enterobacter amnigenus, Enterobacter arachidis, Enterobacter asburiae, Enterobacter carcinogenus, Enterobacter cloacae, Enterobacter cowanii, Enterobacter dissolvans, Enterobacter gergoviae, Enterobacter helveticus, Enterobacter hormaechei, Enterobacter kobei, Enterobacter ludwigii, Enterobacter mori, Enterobacter nimipressuralis, Enterobacter oryzae, Enterobacter pulveris, Enterobacter pyrinus, Enterobacter radicincitans, Enterobacter soli, Enterobacter taylorae, Enterobacter turicensis, Morganella morganii, Citerobacter freundii, Citerobacter koseri, Citerobacter amalonaticus, Citerobacter farmeri, Citerobacter youngae, Citerobacter braakii, Citerobacter werkmanii, Citerobacter sedlakii, Citerobacter rodentium, Citerobacter gillenii, or Citerobacter murliniae.

23. A method of preventing, reducing the risk of, delaying the onset of, and/or reducing the severity of the systemic spread of bacteria to one or more major organ systems of an individual, comprising the step of administering to the individual an effective amount of a composition comprising the non-acylated/inactive form of HlyA and/or a non-acylated/inactive form or functional fragment thereof or a functional fragment thereof.

24. A method of reducing the bacterial burden of an individual, comprising the step of administering to the individual an effective amount of a composition comprising the non-acylated/inactive form of HlyA and/or a non-acylated/inactive form or functional fragment thereof

25. A method of preventing, reducing the risk of, delaying the onset of, and/or reducing the severity of bacteremia in an individual, comprising the step of administering to the individual an effective amount of a composition comprising the non-acylated/inactive form of HlyA and/or a non-acylated/inactive form or functional fragment thereof.

26. A method of preventing, reducing the risk of, delaying the onset of, and/or reducing the severity of a urinary tract infection in an individual, comprising the step of administering to the individual an effective amount of a composition comprising the non-acylated/inactive form of HlyA and/or a non-acylated/inactive form or functional fragment thereof.

27. A method of preventing, reducing the risk of, delaying the onset of, and/or reducing the severity of colonization of bacteria from the Gammaproteobacteria Class in an individual, comprising the step of administering to the individual an effective amount of a composition comprising the non-acylated/inactive form of HlyA and/or a non-acylated/inactive form or functional fragment thereof.

28. A method of preventing, reducing the risk of, delaying the onset of, and/or reducing the severity of sepsis in an individual, comprising the step of administering to the individual an effective amount of a composition comprising the non-acylated/inactive form of HlyA and/or a non-acylated/inactive form or functional fragment thereof.

29. The method of any one of the preceding claims, further comprising the step of administering to the individual an effective amount of a composition comprising SinH or a functional fragment thereof.

30. The method of claim 29, wherein the composition comprising the inactive HlyA or a functional fragment thereof is provided to the individual in the same formulation as the composition comprising the SinH or a functional fragment thereof.

31. The method of claim 29, wherein the composition comprising the inactive HlyA or a functional fragment thereof is provided to the individual in a different formulation as the composition comprising the SinH or a functional fragment thereof.

32. The method of claim 31, wherein the composition comprising the inactive HlyA or a functional fragment thereof is administered to the individual prior to administering the composition comprising the SinH or a functional fragment thereof.

33. The method of claim 31, wherein the composition comprising the inactive HlyA or a functional fragment thereof is administered to the individual subsequent to administering the composition comprising the SinH or a functional fragment thereof.

34. The method of claim 31, wherein the composition comprising the inactive HlyA or a functional fragment thereof is administered to the individual at the same time as administering the composition comprising the SinH or a functional fragment thereof.

35. The method of any one of the preceding claims, wherein the bacteria is drug-resistant.

36. The method of any one of the preceding claims, wherein the bacteria is multidrug-resistant.

37. The method of any one of the preceding claims, wherein the bacteria is E. coli and the E. coli is ST73, ST95, or ST131.

38. The method of any one of the preceding claims, wherein the bacteria is extraintestinal pathogenic Escherichia coli (ExPEC).

39. The method of any one of the preceding claims, wherein the functional fragment of SinH comprises extracellular domain 1, domain 2, and/or domain 3.

40. The method of any one of the preceding claims, wherein the infection is local.

41. The method of any one of the preceding claims, wherein the infection is systemic.

42. The method of any one of the preceding claims, wherein the individual is immunocompromised.

43. The method of any one of the preceding claims, wherein the individual is an infant, child, adolescent, or adult.

44. The method of any one of the preceding claims, wherein the individual is at least 10, 20, 30, 40, 50, 60, 65, 70, 75, 80, 85, 90, 95, or 100 or more years of age.

45. The method of any one of the preceding claims, wherein the individual has recurrent urinary tract infections.

46. The method of any one of the preceding claims, wherein the individual is in a medical facility or has been or will be in a medical facility within 1, 2, 3, 4, 5, 6, or 7 days, or within 1, 2, 3, 4 week, or within 1, 2, 3, 4, 5, 6, 7, 8, 9, 10, 11, or 12 months, or within 1, 2, 3, 4, 5, 6, 7, 8, 9, 10, or more years.

47. The method of claim 46, wherein the medical facility is a hospital or nursing home or skilled nursing home or long-term care facility.

48. The method of any one of the preceding claims, wherein the individual lacks a pathogenic E. coli infection.

49. The method of any one of the preceding claims, wherein the individual has a pathogenic E. coli infection.

50. The method of any one of the preceding claims, wherein the administering step occurs once.

51. The method of any one of claims 1-50, wherein the administering step occurs more than once.

52. The method of claim 51, wherein the duration between successive administrations is, or is at least, or is no more than within 1, 2, 3, 4, 5, 6, or 7 days, or within 1, 2, 3, 4 week, or within 1, 2, 3, 4, 5, 6, 7, 8, 9, 10, 11, or 12 months, or within 1, 2, 3, 4, 5, 6, 7, 8, 9, 10, or more years.

53. The method of any one of the preceding claims, wherein the composition comprises an adjuvant.

54. The method of any one of the preceding claims, wherein the SinH functional fragment is part of a conjugate, wherein the inactive HlyA functional fragment is part of a conjugate, or both.

55. A composition comprising a non-acylated/inactive form alpha-hemolysin (HlyA) and/or a non-acylated/inactive form or functional fragment thereof in a pharmaceutically acceptable excipient.

56. The composition of claim 55, further comprising an antibiotic, SinH, the extracellular domain 1 of SinH, the extracellular domain 2 of SinH, the extracellular domain 3 of SinH, one or more O-antigens, one or more K-antigens, and/or FimH (Type 1 fimbriae protein).

57. A kit comprising the composition of claim 55 or 56, housed in a suitable container.
